(19) United States
(12) Patent Application Publication
(10) Pub. No.: US 2024/0293405 A1
(43) Pub. Date: Sep. 5, 2024

(54) (1H-PYRROLO[2,3-B]PYRIDIN-1-YL)PYRIMIDIN-2-YL-AMINO-PHENYL-ACRYLAMIDE INHIBITORS OF EGFR FOR USE IN THE TREATMENT OF BRAIN TUMORS

(71) Applicants: Jean ZHAO, Brookline, MA (US); William KERNS, Evergreen, CO (US); Dana-Farber Cancer Institute, Inc., Boston, MA (US); Crimson Biopharm Inc., Brookline, MA (US)

(72) Inventors: Jean J. Zhao, Brookline, MA (US); William Kerns, Evergreen, CO (US)

(21) Appl. No.: 18/573,473

(22) PCT Filed: Jun. 22, 2022
(86) PCT No.: PCT/US22/34574

§ 371 Date: Dec. 22, 2023

Related U.S. Application Data
(60) Provisional application No. 63/257,907, filed on Oct. 20, 2021.
(60) Provisional application No. 63/213,301, filed on Jun. 22, 2021.

(51) Int. Cl. A61K 31/506; C07D 471/04

(52) U.S. Cl. CPC A61K 31/506; C07D 471/04

(57) ABSTRACT

The invention generally relates to pharmaceuticals and therapeutic methods. More particularly, the invention provides small molecule EGFR inhibitors (e.g., EGFR tyrosine kinase inhibitor compounds) and pharmaceutical compositions thereof, as well as methods of their use in treating various diseases and conditions, such as cancers of the central nervous system (e.g., primary and metastatic brain cancers) and lung cancers.

### CROSS-REFERENCE TO RELATED APPLICATIONS

This application claims the benefit of priority to U.S. Provisional Patent Application No. 63/213,301, filed Jun. 22, 2021, and U.S. Provisional Patent Application No. 63/257,907, filed Oct. 20, 2021. The contents of each of these applications are hereby incorporated herein by reference in their entirety.

### BACKGROUND

Glioblastoma (GBM) is the most common and malignant primary brain tumor in adults. Many targeted therapies have demonstrated extensive success in other cancer types but have limited efficacy in GBM, and the prognosis for patients with GBM remains grim.

More than 50% of glioblastomas have aberrant EGFR genetic variants. Most of these EGFR variants occur through mutations in the extracellular domain. Among them, the most common EGFR variant (v), EGFRvIII (deletion of exon 2-7), has an in-frame extracellular domain truncation. It has been shown that EGFR-mutant GBM cells are likely addicted to EGFR signaling. Therefore, EGFR is an attractive therapeutic target in GBM.

Currently, there are five EGFR tyrosine kinase inhibitors (TKIs; gefitinib, erlotinib, afatinib, dacomitinib and osimertinib) approved by the Food and Drug Administration (FDA) for the treatment of EGFR-mutant lung cancer in the United States. Gefitinib and erlotinib are first-generation EGFR-TKIs that inhibit catalytic activity by competing with ATP for binding to the ATP-binding site on the kinase domain. Administration of gefitinib or eroltinib results in significantly improved survival of patients over platinum chemotherapy. The second generation of EGFR inhibitors, afatinib and dacomitinib, irreversibly inhibit all four ErbB receptors, including EGFR. As such, they are more potent inhibitors of EGFR but with increased toxicity. Osimertinib, the only FDA-approved third-generation EGFR-TKI, is a covalent inhibitor designed to target EGFR resistance mutations that emerge with EGFR-TKI treatment.

While these first- and second-generation EGFR-TKIs have been shown to inhibit proliferation of GBM cells in preclinical experiments, they have not been effective in the clinic for GBM patients. There are two principal reasons for their failures. First, the first- and second-generation EGFR-TKIs do not cross the blood-brain barrier (BBB). Third-generation EGFR-TKI osimertinib, on the other hand, has been reported to have activity against brain metastases of lung cancer with EGFR mutations and has higher brain penetration, has been proposed for the treatment of EGFR-mutant GBMs. Second, dose-limiting toxicity (DLT) may prevent the approved EGFR-TKIs from being a safe and effective drug for patients with GBM. Unlike EGFR mutations in lung cancers, such as exon-19 deletion, or L858R and T790M substitutions, which reside in the intracellular kinase domain (KD), the common feature of EGFR variants in GBM is a mutant extracellular domain with a wild type (WT) intracellular KD. Because of these complications, it has thus far been impossible to design a true targeted therapeutic that suppresses EGFR signaling within central nervous system (CNS) tumors at concentrations that spare systemic WT EGFR function in vivo.

### SUMMARY

In certain aspects, the present disclosure provides methods of treating glioblastoma multiforme, astrocytoma, congenital tumor of the brain, ependymoma, germinoma, glioma, gliomatosis, gliosarcoma, medulloblastoma, meningioma, meningiosarcoma, oligodendroglioma, pinealoma, retinoblastoma, schwannoma, or spinal cord neurofibroma, comprising administering to a human subject in need thereof a therapeutically effective amount of a compound of Formula I:

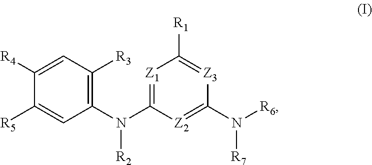

or pharmaceutically acceptable salts, hydrates, solvates, stereoisomers, or tautomers thereof, wherein the therapeutically effective amount is at least 100 mg/day, and wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of

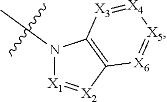

wherein
   X₃ is N;
X₁, X₂, X₄, X₅ and X₆ are each independently CH or CR₁₅; and
each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, the method further comprises examining the skin of the subject within 1 month after administration, wherein the subject does not exhibit skin lesions within 1 month after administration.

In certain embodiments, the method is a method of treating glioblastoma multiforme. In certain embodiments, the compound of Formula I is characterized by a binding affinity for EGFR and/or mutated EGFR in the subject of no more than 10 nM.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In some embodiments, the subject does not lose more than 10% of its body weight within 1 month after administration. The therapeutically effective amount of the compound can be administered daily to the subject for at least 1 month. The therapeutically effective amount can be from 100 mg/day to 1000 mg/day, from 100 mg/day to 800 mg/day, from 100 mg/day to 500 mg/day, and/or from 200 mg/day to 500 mg/day.

The methods can further involve examining the skin of the subject within 1 month after administration, wherein the subject does not exhibit skin lesions within 1 month after administration. In some embodiments, the methods further involve examining the skin of the subject within 2 months after administration, wherein the subject does not exhibit skin lesions within 2 months after administration.

In some embodiments, these methods are a method of treating glioblastoma multiforme. The glioblastoma multiforme can be characterized by elevated levels of EGFR and/or mutated EGFR. In some embodiments, the compound of Formula I is not a substrate of an efflux transporter. In some embodiments, the compound of Formula I is characterized by a binding affinity for EGFR and/or mutated EGFR in the subject of no more than 10 nM, such as no more than 9 nM, no more than 8 nM, no more than 7 nM, no more than 6 nM, no more than 5 nM, no more than 4 nM, no more than 3 nM, no more than 2 nM, no more than 1 nM, no more than 0.9 nM, no more than 0.8 nM, no more than 0.7 nM, no more than 0.6 nM, no more than 0.5 nM, no more than 0.4 nM, no more than 0.3 nM, no more than 0.2 nM, no more than 0.15 nM, no more than 0.12 nM, no more than 0.11 nM, or no more than 0.10 nM.

These methods can be a method of treating an astrocytoma.

In some embodiments, Z₁ and Z₂ are each N and Z₃ is CR₈, R₁ is H or NH₂, R₂ is H, R₃ is (C₁-C₄) alkoxy, R₄ is NR₉R₁₀, R₅ is NR₁₂C(O)R₁₃, and/or R₁₅ is selected from (C₁-C₆) alkyl and (C₁-C₆) haloalkyl and/or is selected from methyl and CF₃.

In some embodiments, R₈ is H or halogen, R₉ is (C₁-C₄) alkyl, and/or R₁₀ is (C₁-C₄) alkyl-NH(C₁-C₄) alkyl, or (C₁-C₄) alkyl-N((C₁-C₄) alkyl)₂. In some embodiments, R₄ is NR₉R₁₀ and R₉ and R₁₀ together with the nitrogen atom to which they are attached form a 5-to 7-membered heterocycle optionally comprising 1 or 2 additional heteroatoms selected from N, O, and S and optionally substituted with one or more R₁₁. In some embodiments, R₁₁ is (C₁-C₄) alkyl, R₁₂ is H, and R₁₃ is (C₂-C₆) alkenyl. In some embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

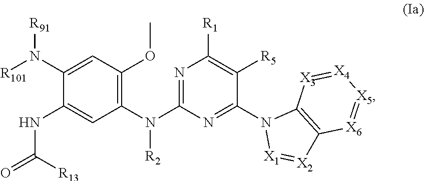

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5-to 7-membered heterocycle optionally comprising 1 or 2 additional heteroatoms selected from N, O, and S and optionally substituted with one or more R₁₁.

In still another embodiment, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In some embodiments the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

The compound can be administered once per day, two times per day, or three times per day.

In some embodiments, the compound is administered systemically. In other embodiments, the compound is administered orally. In still other embodiments, the compound is administered intravenously.

In some aspects, the present disclosure provides methods for treating or reducing a brain tumor, or a related disease or condition, comprising administering to a subject in need thereof a therapeutically effective amount of a compound having the formula of Compound 1:

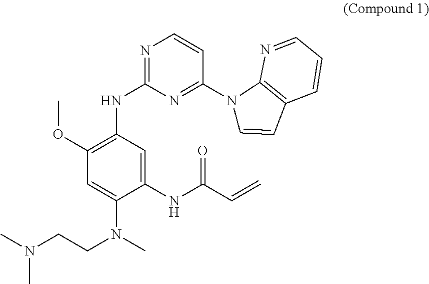 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In some aspects, the present disclosure provides methods for inhibiting or reducing the activity of epidermal growth factor receptor (EGFR) in a subject suffering from a brain tumor, comprising administering to the subject a therapeutically effective amount of a compound having the formula of Compound 1:

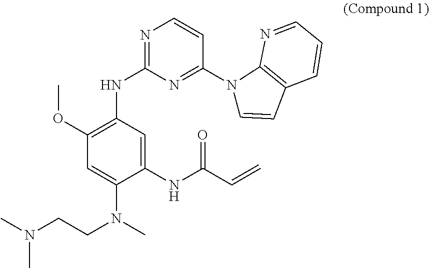 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In some aspects, the present disclosure provides methods for treating or reducing a brain disease or condition mediated by epidermal growth factor receptor (EGFR), comprising administering to the subject in need thereof a therapeutically effective amount of a compound having the formula of Compound 1:

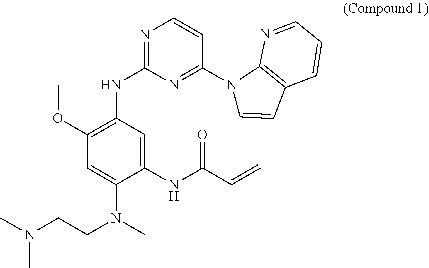 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In some embodiments, the brain tumor comprises a primary tumor. In other embodiments, the brain tumor comprises a metastatic tumor. In certain embodiments, the brain tumor is glioblastoma.

In some embodiments, the therapeutically effective amount is in the range from about 0.1 to about 20 mg/kg body weight daily. In some such embodiments, the therapeutically effective amount is in the range from about 0.5 to about 5 mg/kg body weight daily.

In other aspects, the invention generally relates to a pharmaceutical composition for treating a brain tumor, or a related disease or condition, comprising a compound having the formula of Compound 1:

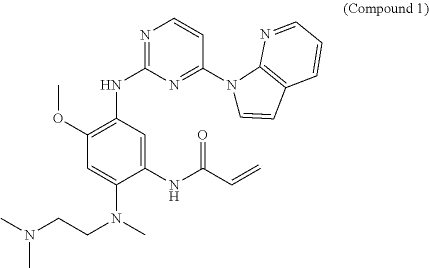 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In some embodiments, the brain tumor comprises a primary tumor, such as glioblastoma. In other aspects, the brain tumor comprises a metastatic tumor.

In yet other aspects, the present disclosure provides a compound having the structural formula of Compound 2:

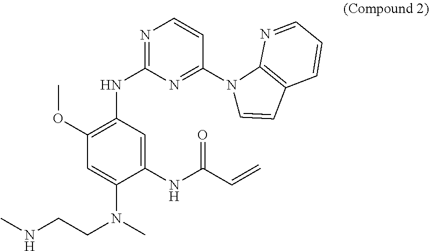 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In yet other aspects, the present disclosure provides pharmaceutical compositions comprising a compound having the structural formula of Compound 2

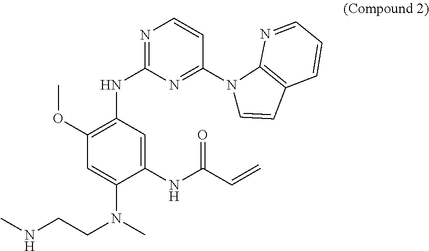 [SMILES: C=CC(=O)Nc1cc(Nc2nccc(-n3ccc4cccnc43)n2)c(OC)cc1N(C)CCNC]

or a pharmaceutically acceptable form or an isotope derivative thereof, and a pharmaceutically acceptable excipient, carrier, or diluent. In certain embodiments, the pharmaceutical composition is suitable for oral administration. In other embodiments, the pharmaceutical composition is suitable for intravenous administration.

In some embodiments, the pharmaceutical composition is suitable for use in treating a disease or condition selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas and myelomas.

In yet other aspects, the present disclosure provides a unit dosage form comprising a pharmaceutical composition disclosed herein.

In yet other aspects, the present disclosure provides methods for treating or reducing a disease or condition, comprising administering to a subject in need thereof a therapeutically effective amount of a compound having the formula of Compound 2:

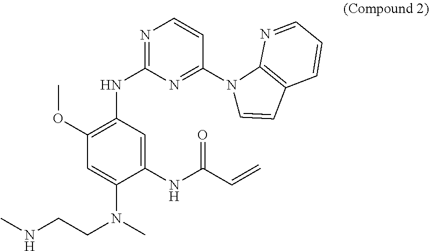 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In yet other aspects, the present disclosure provides methods for inhibiting or reducing the activity of EGFR in a subject suffering from a disease or condition related thereto, comprising administering to a subject in need thereof a therapeutically amount of a compound having the formula of Compound 2:

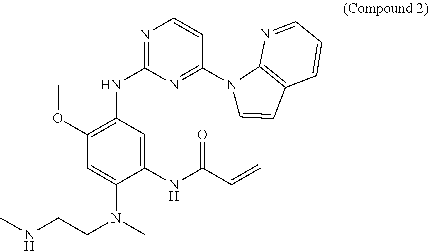 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In yet other aspects, the present disclosure provides methods for treating or reducing a disease or condition mediated by EGFR, comprising administering to the subject in need thereof a therapeutically effective amount of a compound having the formula of Compound 2:

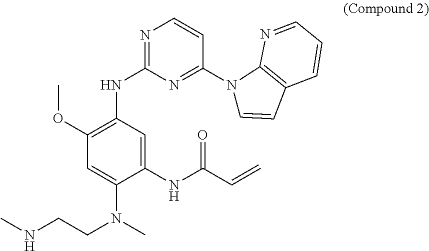 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In some embodiments, the disease or condition is a cancer, such as a cancer selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas and myelomas. In some such embodiments, the cancer comprises a primary tumor. In other such embodiments, the cancer comprises a metastatic tumor. In still other such embodiments, the cancer is glioblastoma. In even still other such embodiments, the cancer is lung cancer, such as non-small cell lung cancer (NSCLC) or small cell lung cancer (SCLC).

In certain embodiments, the subject carries an EGFR mutation, such as a T790M EGFR mutation.

In some embodiments, the therapeutically effective amount is in the range from about 0.1 to about 20 mg/kg body weight daily, such as wherein the therapeutically effective amount is in the range from about 0.5 to about 5 mg/kg body weight daily.

In yet other aspects, the present disclosure provides methods for treating or reducing a brain tumor, or a related disease or condition, comprising administering to a subject in need thereof a therapeutically effective amount of a compound having the formula of Compound 2:

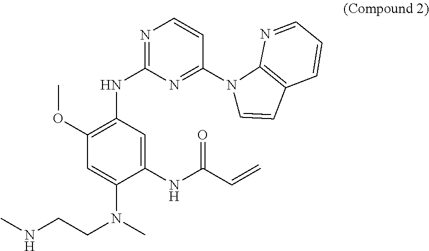 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In yet other aspects, the present disclosure provides methods for inhibiting or reducing the activity of EGFR in a subject suffering from a brain tumor, comprising administering to a subject in need thereof a therapeutically amount of a compound having the formula of Compound 2:

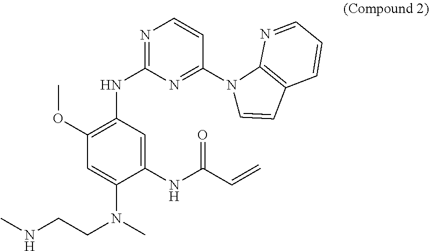 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In still other aspects, the present disclosure provides methods for treating or reducing a brain disease or condition mediated by EGFR, comprising administering to the subject in need thereof a therapeutically effective amount of a compound having the formula of Compound 2:

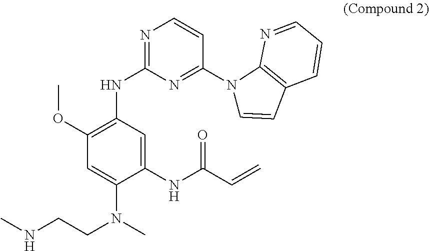 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In some embodiments, the brain tumor comprises a primary tumor. In other embodiments, the brain tumor comprises a metastatic tumor. In certain embodiments, the brain tumor is glioblastoma.

In yet other aspects, the present disclosure provides use of a compound or a pharmaceutical composition thereof for treating or reducing a brain tumor, or a related disease or condition, wherein the compound has the formula of Compound 1:

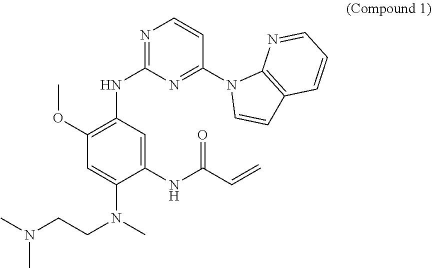 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In yet other aspects, the invention generally relates to use of a compound or a pharmaceutical composition thereof for inhibiting or reducing the activity of EGFR in a subject suffering from a brain tumor, wherein the compound has the formula of Compound 1:

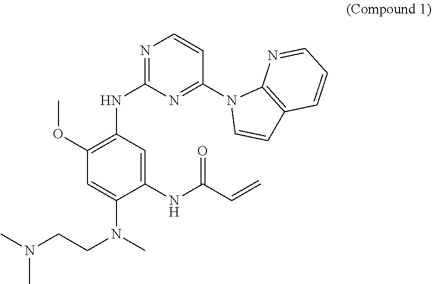 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In yet other aspects, the present disclosure provides use of a compound or a pharmaceutical composition thereof for treating or reducing a brain disease or condition mediated by EGFR, comprising administering to the subject in need thereof a therapeutically effective amount of a compound having the formula of Compound 1:

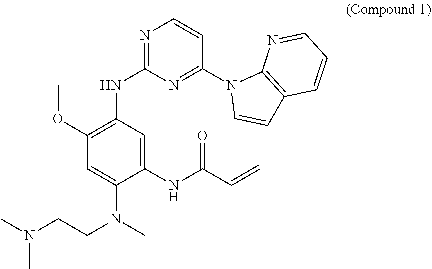 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In some embodiments, the brain tumor comprises a primary tumor. In other embodiments, the brain tumor comprises a metastatic tumor. In still other embodiments, the brain tumor is glioblastoma.

In yet other aspects, the present disclosure provides uses of a compound or a pharmaceutical composition thereof for treating or reducing a disease or condition, wherein the compound has the formula of Compound 2:

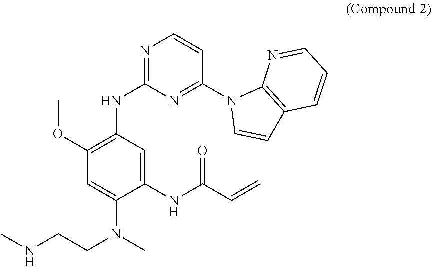 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In yet other aspects, the present disclosure provides uses of a compound or a pharmaceutical composition thereof for inhibiting or reducing the activity of EGFR in a subject suffering from a disease or condition related thereto, wherein the compound has the formula of Compound 2:

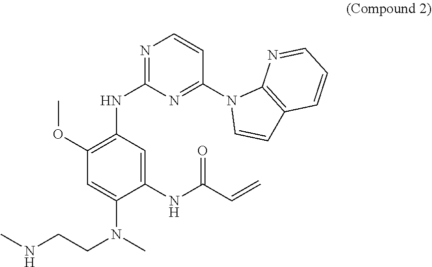 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In yet other aspects, the present disclosure provides uses of a compound or a pharmaceutical composition thereof for treating or reducing a disease or condition mediated by EGFR, wherein the compound has the formula of Compound 2:

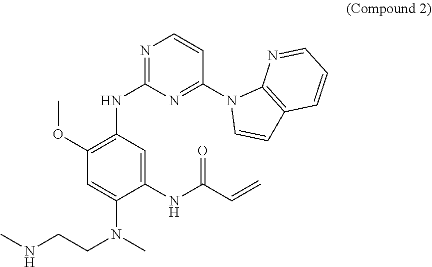 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In some embodiments, the disease or condition is a cancer, such as a cancer selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas and myelomas. In some such embodiments, the brain tumor comprises a primary tumor. In other such embodiments, the brain tumor comprises a metastatic tumor. In still other such embodiments, the cancer is glioblastoma. In yet other such embodiments, the cancer is lung cancer, such as non-small cell lung cancer (NSCLC).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is the western blot analysis with the indicated antibodies after 293-EGFRvIII cells were treated with Compound 1 or erlotinib at the indicated doses for 6 hours.

FIG. 1B is a graph that shows western blot analysis of 293-EGFRvIII cells that were treated with Compound 1 at the indicated doses for 6 hours with antibodies against pEGFRvIII1068 and EGFRvIII to determine IC50 for inhibition of pEGFRvIII1068. α-Tubulin was used as a loading control.

FIG. 1C is a graph showing IC50 (μM) of Compound 1 and other EGFR-TKIs as indicated by the viability of 293-EGFRvIII cells.

FIG. 2A is a graph showing viability of BT122 cells upon treatment with Compound 1 for 3 days in a dose titration from 0.156 μM to 20 μM for each drug. A table is also shown that compiles the IC50 of Compound 1 and other EGFR inhibitors for BT112 cells.

FIG. 2B is a graph showing viability of BT179 cells upon treatment with Compound 1 for 3 days in a dose titration from 0.156 μM to 20 μM for each drug. A table is also shown that compiles the IC50 of Compound 1 and other EGFR inhibitors for BT179 cells.

FIG. 2C is a graph showing viability of BT333 cells upon treatment with Compound 1 for 3 days in a dose titration from 0.156 μM to 20 μM for each drug. A table is also shown that compiles the IC50 of Compound 1 and other EGFR inhibitors for BT333 cells.

FIG. 3A is a graph showing western blot analysis of U251-EGFRvIII cells were treated with Compound 1 at indicated doses for 20 hours with the antibodies against pEGFRvIII1068 and EGFRvIII to determine IC50 for inhibition of pEGFRvIII1068. IC50 is 0.174 μM.

FIG. 3B is a graph showing U251-EGFRvIII cells were treated with Compound 1 or other EGFR TKIs as indicated. IC50s of Compound 1 and other EGFR-TKIs are indicated on the table below the curves.

FIG. 3C is a graph showing proliferation of U251 cells upon treatment with Compound 1 and other EGFR inhibitors for 3 days in a dose titration from 0.156 μM to 20 μM for each drug. A table is also shown that compiles the IC50 of Compound 1 and other EGFR inhibitors for U251 cells.

FIG. 4A is a set of representative bioluminescence images of a first cohort of mice bearing U251-EGFRvIII at indicated weeks after treatment with vehicle control (n=3), Compound 1 at 37.5 mg/kg (QD, n=3) or 75 mg/kg (QD, n=2).

FIG. 4B is a bar graph showing quantification of the regions of interest (ROI) in each mouse in the first cohort after treatment for four weeks compared to week zero, which was set as baseline.

FIG. 4C is a set of representative bioluminescence images of a second cohort of mice bearing U251-EGFRvIII at indicated times (weeks) after treatment with control (n=4), Compound 1 at 37.5 mg/kg (QD, n=5) or 75 mg/kg (QD, n=5).

FIG. 4D is bar graph showing quantification of the regions of interest (ROI) in each mouse in the second cohort after treatment for four weeks compared to week zero, which was set as baseline.

FIG. 4E is a Kaplan-Meier survival analysis of U251-EGFRvIII xenograft-bearing mice from both cohorts treated with Compound 1 (37.5 mg/kg, PO QD, n=8), Compound 1 (75 mg/kg, PO QD, n=7), or vehicle control (n=7). Mean±SD, * p<0.05; ** p<0.01, Log-rank (Mantel-Cox) test.

FIG. 4F is the body weight record of U251-EGFRvIII xenograft-bearing mice from both cohorts treated with Compound 1 (37.5 mg/kg, PO QD, n=8), Compound 1 (75 mg/kg, PO QD, n=7), or vehicle control (black line, n=7). Mean±SD, * p<0.05; ** p<0.01, Log-rank (Mantel-Cox) test.

FIG. 5A shows a representation of the genetic alterations in the syngeneic genetically engineered mouse model of glioblastoma driven by Cdkn2a^{null}; Pten^{null}; hEGFRvIII described in Example 5.

FIG. 5B is the western blot analysis of primary mouse CPEvIII cells that were treated with Compound 1 for 1 day.

FIG. 5C is a Kaplan-Meier survival analysis of tumor-bearing mice treated with Compound 1 (75 mg/kg, PO QD, n=5) or vehicle control (n=6) *p<0.05 (p=0.017), Logrank (Mantel-Cox) test.

FIG. 5D is an IHC analysis of CPEvIII tumors collected at end point from mice treated with Compound 1 or vehicle control. Scale bar, 100 μm.

FIG. 5E is a graph showing the body weight of tumor-bearing mice treated with Compound 1 or vehicle control.

FIG. 6A shows mice treated with 10-25 mg/kg/day osimertinib.

FIG. 6B shows mice treated with 10-50 mg/kg/day Compound 1 instead.

FIG. 7 shows change in body weight over the course of treatment for mice treated with Compound 1 (25-50 mg/kg) or osmertinib (25 mg/kg).

FIG. 8 shows body weight loss in female SCID mice bearing NSCLC brain metastases dosed with Compound 1 or osimertinib as described in Example 11.

FIG. 9 shows suppression of brain metastases by both compounds, as described in Example 11.

FIG. 10A is bioluminescence images of GBM-bearing mice dosed with 100 mg/kg Compound 1 administered orally, or control mice, sacrificed 7 hours post-treatment.

FIG. 10B is images of brain tissues derived from control and Compound 1-treated mice stained with hematoxylin and eosin (H&E).

FIG. 10C shows control mimetic plated onto a MALDI substrate along with brain tissue sections from a control and a Compound 1-treated mouse.

FIG. 10D shows the signal observed from the mimetic samples during the MALDI-MSI analysis.

FIG. 10E shows the normalized curve generated from the intensities observed for different concentrations of the mimetic plated on the MALDI substrate.

FIG. 10F is an image of the intensities observed for the brain tissue samples.

FIG. 10G shows the absolute intensities observed from the MALDI analysis of the mimetics.

FIG. 10H show the absolute intensities observed from the MALDI analysis of the brain tissue sample derived from the Compound 1 treated mouse.

FIG. 10I shows an MSMS analysis of Compound 1.

FIG. 11 shows kinase profiling results using AZD9291, Compound 1, and Compound 2 discussed in Example 18.

### DETAILED DESCRIPTION

The present application relates to small molecule EGFR-TKI compounds and pharmaceutical compositions thereof, as well as methods of their use in treating various diseases and conditions, such as cancers, optionally cancers of the central nervous system (CNS) and lung cancers. In some non-limiting examples, the cancer is a primary or metastatic brain cancer. In other non-limiting examples, the cancer is a cancer of the CNS, such as a glioblastoma (GBM), such as an adult GBM with aberrant EGFR. In still other non-limiting examples, the cancer is a lung cancer, such as non-small cell lung cancer (NSCLC) or small cell lung cancer (SCLC).

The present application is based in part on the discovery of novel therapeutic agents, compositions and methods for treating various diseases and conditions, including primary brain cancer (e.g., GBM), metastatic brain cancers, and lung cancers. In particular, the present invention provides Compound 11 and Compound 12, shown below, and compositions and methods of use thereof, for treating GBMs and other cancers with aberrant EGFR. Importantly, both Compound 11 and Compound 12 have shown favorable pharmacokinetic (PK) and safety profiles with extraordinary brain-specific distribution and accumulation. In the case of Compound 11, the brain-to-plasma ratio was shown to be greater than 20-fold at estimated steady state, in sharp contrast from other reported EGFR inhibitors.

In certain embodiments, described herein are methods of treating cancers of the central nervous system (CNS), for example GBMs, such as adult GBMs with aberrant EGFR, using a covalent-binding EGFR-TKI, Compound 1. Pre-clinical efficacy studies showed that Compound 1 is more effective than other EGFR-TKIs in blocking the proliferation of GBM tumor cells from both patient-derived and cultured human GBM cell lines with EGFR amplification and/or EGFRvIII mutation. In addition, Compound 1 administered as a single agent was able to attenuate the growth of orthotopic U251-EGFRvIII xenografts and extend the survival of tumor-bearing mice in a dose-dependent manner. Moreover, Compound 1 inhibited EGFR phosphorylation in GBM tumors derived from a novel genetically engineered mouse (GEM) model of GBM with EGFRvIII expression both in vitro and in vivo. Compound 1 also extended the survival of mice bearing orthotopic allografts of GBM. Notably, mice maintained stable body weight during treatments with increasing doses of Compound 1 up to 75 mg/kg per day.

In certain embodiments, Compound 1 has more favorable pharmacokinetic (PK) and safety profiles than other reported EGFR inhibitors.

In certain aspects, the present disclosure provides compounds having the structure of Compound 2:

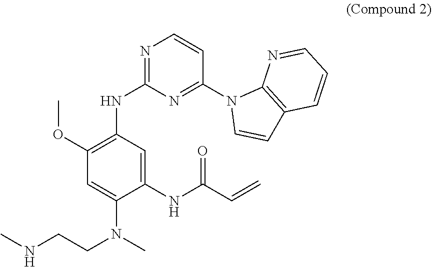 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain aspects, the present disclosure provides methods of treating glioblastoma multiforme, astrocytoma, congenital tumor of the brain, ependymoma, germinoma, glioma, gliomatosis, gliosarcoma, medulloblastoma, meningioma, meningiosarcoma, oligodendroglioma, pinealoma, retinoblastoma, schwannoma, or spinal cord neurofibroma, comprising administering to a human subject in need thereof a therapeutically effective amount of a compound of Formula I:

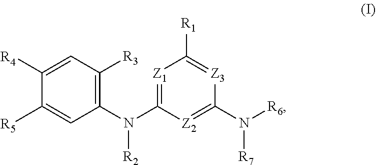

or pharmaceutically acceptable salts, hydrates, solvates, stereoisomers, or tautomers thereof, wherein the therapeutically effective amount is at least 100 mg/day, and wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂; or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

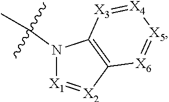

wherein
X₃ is N;
X₁, X₂, X₄, X₅ and X₆ are each independently CH or CR₁₅; and each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, the subject does not lose more than 10% of its body weight within 1 month after administration. In other embodiments, the therapeutically effective amount of the compound is administered daily to the subject for at least one month, and the subject does not lose more than 10% of its body weight within 1 month after daily administration. Changes in subject body weight may be measured by any suitable means known in the art.

In certain embodiments, the therapeutically effective amount is from 100 mg/day to 1000 mg/day, such as from 100 mg/day to 800 mg/day, from 100 mg/day to 500 mg/day, or from 200 mg/day to 500 mg/day.

In certain embodiments, the method further comprises examining the skin of the subject within 1 month after administration, wherein the subject does not exhibit skin lesions within 1 month after administration. In other embodiments, the method further comprises examining the skin of the subject within 2 months after administration, wherein the subject does not exhibit skin lesions within 2 months after administration. As used herein, “examining the skin” of a subject may include visual observation by the subject themselves and/or a medical professional. A subject does not exhibit skin lesions if no skin lesions are observed when the skin is examined visually by the subject themselves and/or a medical professional.

In certain embodiments, the method is a method of treating glioblastoma multiforme. In some such embodiments, the glioblastoma multiforme is characterized by elevated levels of EGFR and/or mutated EGFR. The level of EGFR in a subject (such as in a tumor of the subject) is elevated if it is above the EGFR level in a healthy subject. EGFR is mutated if its amino acid sequence differs from that of wild-type EGFR. EGFR mutations associated with glioblastoma multiforme include those that have been observed in the art and include, but are not limited to, EGFRvIII, EGFR amplification, EGFR missense mutations, and EGFR polysomy. In other such embodiments, the compound of Formula I is not a substrate of an efflux transporter. Efflux transporters are known in the art and include, but are not limited to, P-gp and Bcrp. A compound is not a substrate of an efflux transporter if it does not bind to an efflux transporter with a binding affinity of greater than 10 μM.

In some embodiments, the compound of Formula I is characterized by a binding affinity for EGFR and/or mutated EGFR in the subject of no more than 10 nM, such as no more than 9 nM, no more than 8 nM, no more than 7 nM, no more than 6 nM, no more than nM, no more than 4 nM, no more than 3 nM, no more than 2 nM, no more than 1 nM, no more than 0.9 nM, no more than 0.8 nM, no more than 0.7 nM, no more than 0.6 nM, no more than 0.5 nM, no more than 0.4 nM, no more than 0.3 nM, no more than 0.2 nM, no more than 0.15 nM, no more than 0.12 nM, no more than 0.11 nM, or no more than 0.10 nM. The binding affinity may be determined by any suitable method known in the art.

In other embodiments, the method is a method of treating an astrocytoma.

In certain embodiments, Z₁ and Z₂ are each N and Z₃ is CR₈.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

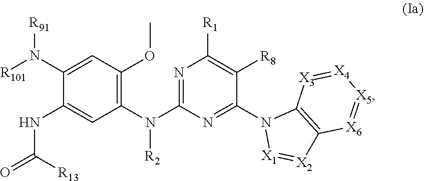

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂; or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the p-toluenesulfonate salt of the compound has the following structure:

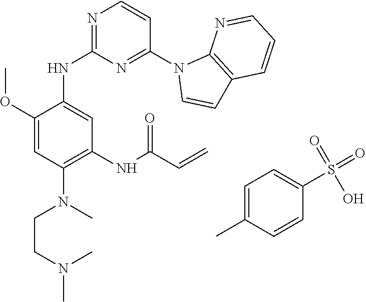 [SMILES: C=CC(=O)Nc1cc(Nc2nccc(-n3ccc4cccnc43)n2)c(OC)cc1N(C)CCN(C)C ; Cc1ccc(S(=O)(=O)O)cc1]

(“Compound 1” tosylate or “Compound 1” p-toluene sulfonate)

In certain embodiments, the compound is the Compound 1:

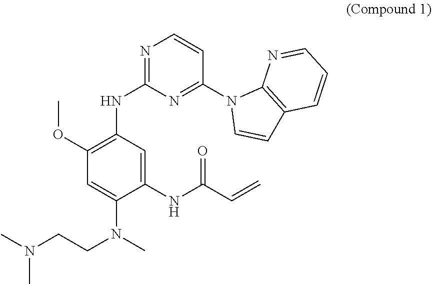 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound is the Compound 2:

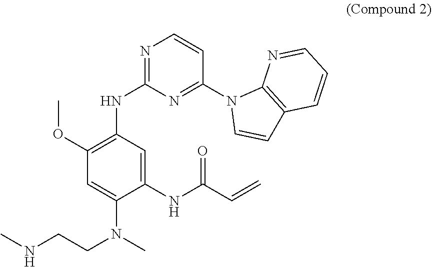 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain embodiments, the compound is administered once per day. In other embodiments, the compound is administered two times per day. In still other embodiments, the compound is administered three times per day.

In certain embodiments, the compound is administered systemically. In some such embodiments, the compound is administered orally. In other such embodiments, the compound is administered intravenously.

In certain aspects, the present disclosure provides methods for treating or reducing a brain tumor, or a related disease or condition, comprising administering to a subject in need thereof a therapeutically effective amount of a compound having the structure of Formula I:

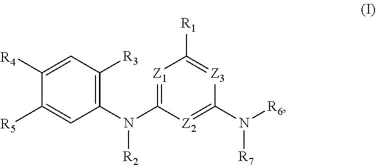

or a pharmaceutically acceptable form or an isotope derivative thereof, wherein:
Z₁, Z₂, and Z₃ are each independently N or CR₈, wherein at least two of Z₁, Z₂, and Z₃ are N;
s R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

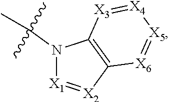

wherein
X₃ is N;
X₁, X₂, X₄, X₅ and X₆ are each independently CH or CR₁₅; and
each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CRS.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

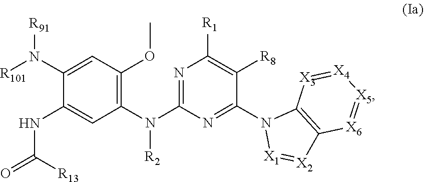

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

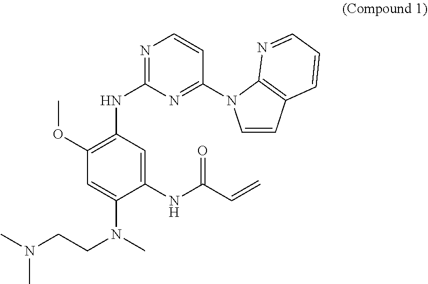 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound of Formula I is Compound 2:

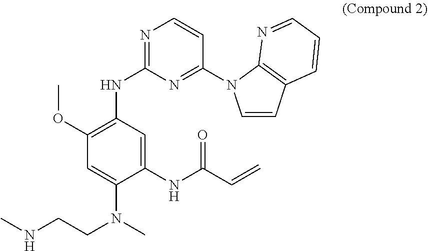 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain embodiments, the brain tumor is glioblastoma.

In certain embodiments, the compound is administered once per day. In other embodiments, the compound is administered two times per day. In still other embodiments, the compound is administered three times per day.

In certain embodiments, the compound is administered systemically. In some such embodiments, the compound is administered orally. In other such embodiments, the compound is administered intravenously.

In other aspects, the present disclosure provides methods for inhibiting or reducing the activity of EGFR in a subject suffering from a brain tumor, comprising administering to the subject a therapeutically effective amount of a compound having the structure of Formula I:

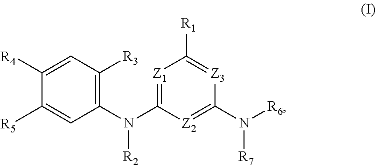

or a pharmaceutically acceptable form or an isotope derivative thereof, wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

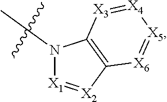

wherein
X₃ is N;
X₁, X₂, X₄, X₅ and X₆ are each independently CH or CR₁₅; and each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CR₈.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

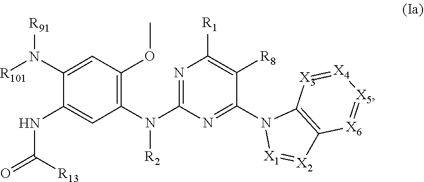

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

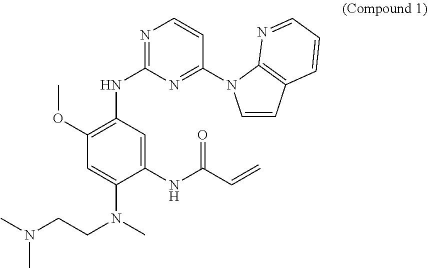 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound of Formula I is Compound 2:

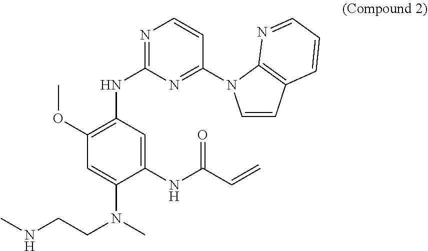 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain embodiments, the brain tumor is glioblastoma.

In certain embodiments, the compound is administered once per day. In other embodiments, the compound is administered two times per day. In still other embodiments, the compound is administered three times per day.

In certain embodiments, the compound is administered systemically. In some such embodiments, the compound is administered orally. In other such embodiments, the compound is administered intravenously.

In other aspects, the present disclosure provides methods for treating or reducing a brain disease or condition mediated by EGFR, comprising administering to the subject in need thereof a therapeutically effective amount of a compound having the structure of Formula I:

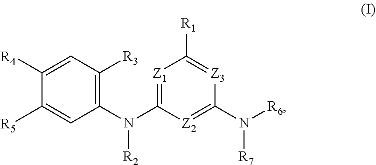

or a pharmaceutically acceptable form or an isotope derivative thereof, wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

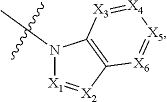

wherein
X₃ is N;
X₁, X₂, X₄, X₅ and X₆ are each independently CH or CR₁₅; and
each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CRS.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

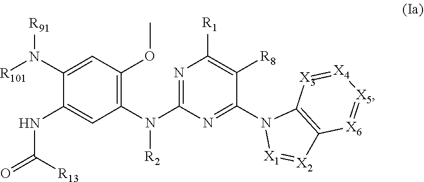

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

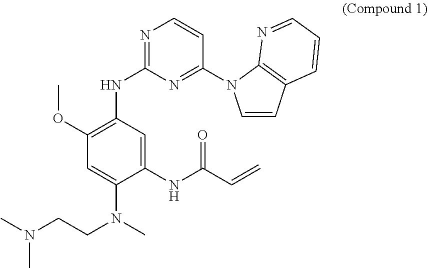 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound of Formula I is Compound 2:

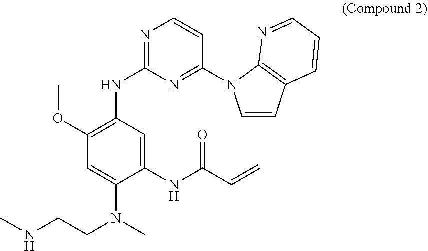 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain embodiments, the brain tumor is glioblastoma.

In certain embodiments, the compound is administered once per day. In other embodiments, the compound is administered two times per day. In still other embodiments, the compound is administered three times per day.

In certain embodiments, the compound is administered systemically. In some such embodiments, the compound is administered orally. In other such embodiments, the compound is administered intravenously.

In other aspects, the present disclosure provides pharmaceutical compositions for treating a brain tumor, or a related disease or condition, comprising a compound having the structure of Formula I:

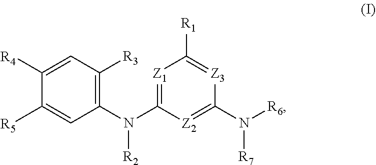

or a pharmaceutically acceptable form or an isotope derivative thereof, and a pharmaceutically acceptable excipient, carrier, or diluent; wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

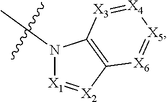

wherein
X₃ is N;
X₁, X₂, X₄, X₅ and X₆ are each independently CH or CR₁₅; and
each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CRS.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

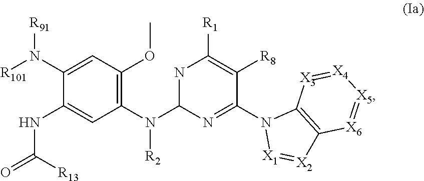

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

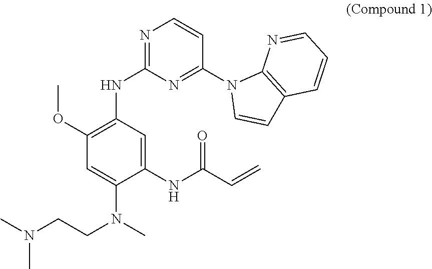 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound of Formula I is Compound 2:

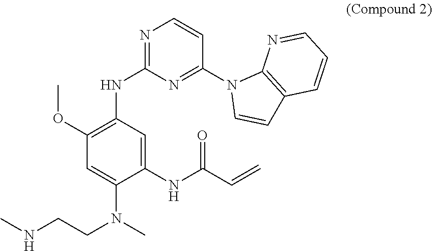 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain embodiments, the brain tumor is glioblastoma.

In other aspects, the present disclosure provides pharmaceutical compositions comprising a compound having the structure of Formula I:

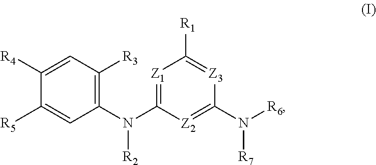

or a pharmaceutically acceptable form or an isotope derivative thereof, and a pharmaceutically acceptable excipient, carrier, or diluent; wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

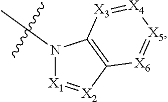

wherein
X₃ is N;
X₁, X₂, X₄, X₅ and X₆ are each independently CH or CR₁₅; and each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CRS.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

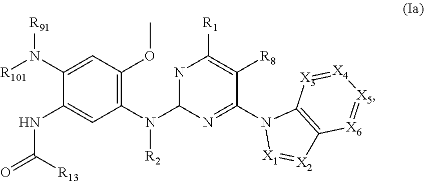

or pharmaceutically acceptable salts, hydrates, solvates, stereoisomers, or tautomers thereof, wherein:
X₁, X₂, X₄, X₅ and X₆ are each independently CR₁₅; R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

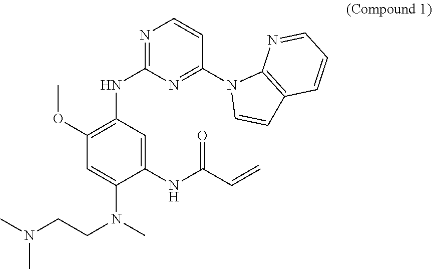 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound of Formula I is Compound 2:

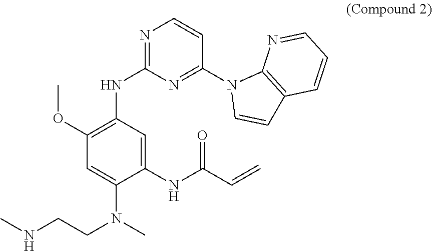 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain embodiments, the pharmaceutical composition is suitable for oral administration.

In certain embodiments, the pharmaceutical composition is suitable for intravenous administration

In certain embodiments, the pharmaceutical composition is suitable for use in treating a disease or condition selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas and myelomas.

In other aspects, the present disclosure provides unit dosage forms comprising a pharmaceutical composition disclosed herein.

In other aspects, the present disclosure provides methods for treating or reducing a disease or condition, comprising administering to a subject in need thereof a therapeutically effective amount of a compound having the structure of Formula I:

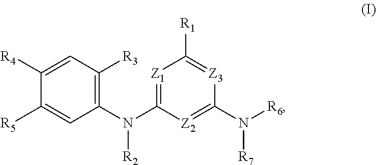

or a pharmaceutically acceptable form or an isotope derivative thereof, wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

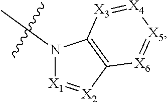

wherein
X₃ is N;
X₁, X₂, X₄, X₅ and X₆ are each independently CH or CR₁₅; and each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CRS.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

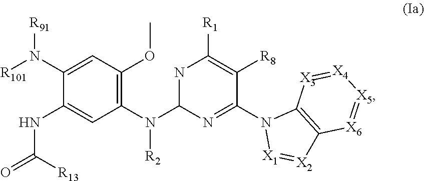

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

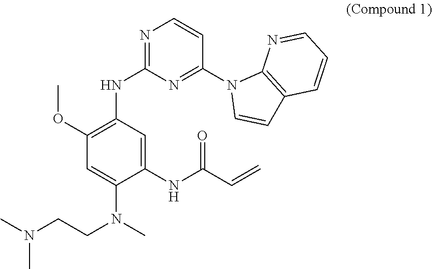 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound of Formula I is Compound 2:

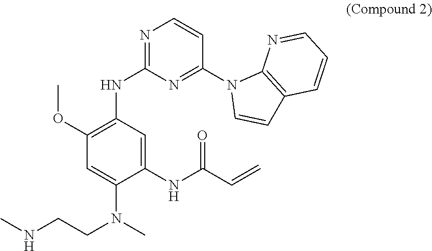 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In other aspects, the present disclosure provides methods for inhibiting or reducing the activity of EGFR in a subject suffering from a disease or condition related thereto, comprising administering to a subject in need thereof a therapeutically effective amount of a compound having the structure of Formula I:

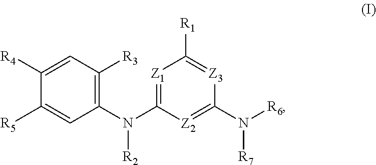

or a pharmaceutically acceptable form or an isotope derivative thereof, wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

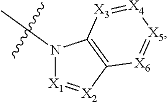

wherein
X₃ is N;
X₁, X₂, X₄, X₅ and X₆ are each independently CH or CR₁₅; and each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CRS.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

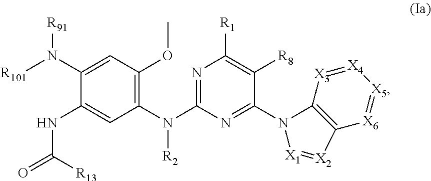

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

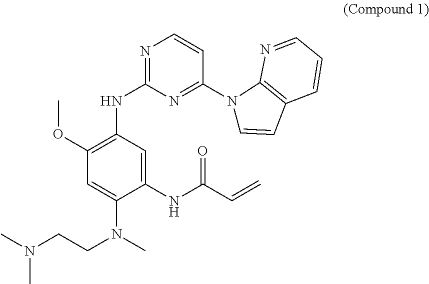 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compund of Formula I is Compound 2:

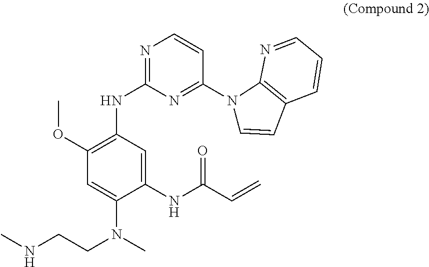 [SMILES: C=CC(=O)Nc1cc(Nc2nccc(-n3ccc4cccnc43)n2)c(OC)cc1N(C)CCNC]

In other aspects, the present disclosure provides methods for treating or reducing a disease or condition mediated by EGFR, comprising administering to the subject in need thereof a therapeutically effective amount of a compound having the structure of Formula I:

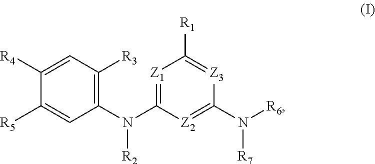

or a pharmaceutically acceptable form or an isotope derivative thereof, wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

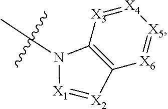

wherein
X₃ is N;
X₁, X₂, X₄, X₅ and X₆ are each independently CH or CR₁₅; and
each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CRS.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

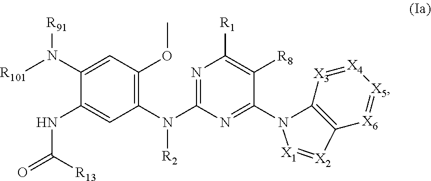

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

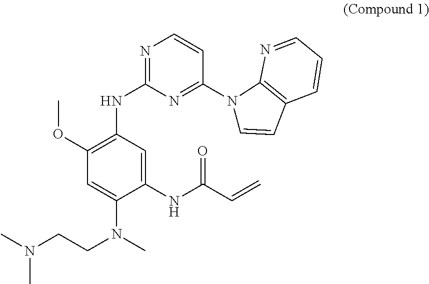 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound of Formula I is Compound 2:

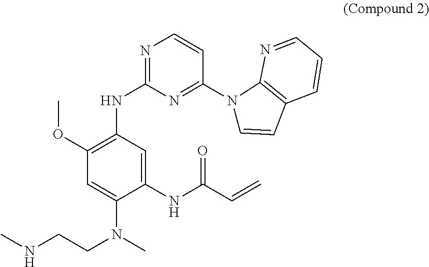 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain embodiments, the disease or condition is a cancer.

In certain embodiments, the cancer is selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas and myelomas.

In certain embodiments, the cancer is glioblastoma.

In certain embodiments, the cancer is lung cancer.

In certain embodiments, the cancer is NSCLC.

In certain embodiments, the cancer is SCLC.

In certain embodiments, the subject carries an EGFR mutation.

In certain embodiments, the subject carries T790M EGFR mutation.

In yet other aspects, the present disclosure provides use of a compound or a pharmaceutical composition thereof for treating or reducing a brain tumor, or a related disease or condition, wherein the compound has the structure of Formula I:

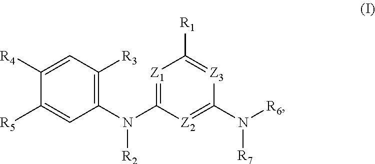

or a pharmaceutically acceptable form or an isotope derivative thereof, wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

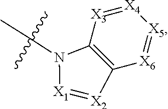

wherein
X₃ is N;
X₁, X₂, X₄, X₅ and X₆ are each independently CH or CR₁₅; and
each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CRS.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

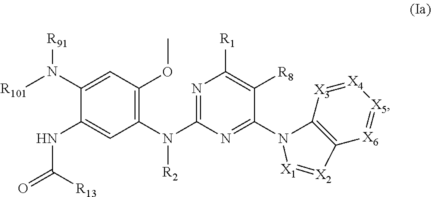

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

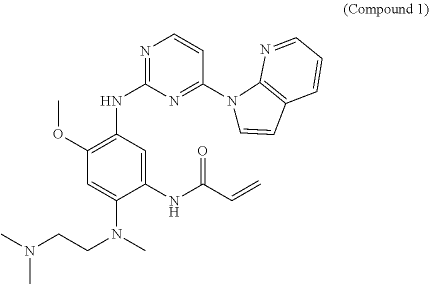 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound of Formula I is Compound 2:

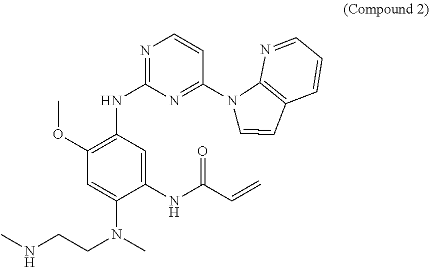 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain embodiments, the use is for treating cancer.

In certain embodiments, the use is for treating a cancer is selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas and myelomas.

In certain embodiments, the use is for treating glioblastoma.

In certain embodiments, the use is for treating lung cancer.

In certain embodiments, the use is for treating non-small cell lung cancer NSCLC.

In other aspects, the present disclosure provides use of a compound or a pharmaceutical composition thereof for inhibiting or reducing the activity of EGFR in a subject suffering from a brain tumor, wherein the compound has the structure of Formula I:

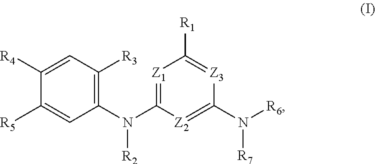

or a pharmaceutically acceptable form or an isotope derivative thereof, wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

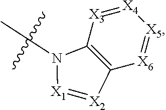

wherein
X₃ is N;
X₁, X₂, X₄, X₅ and X₆ are each independently CH or CR₁₅; and
each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CRS.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

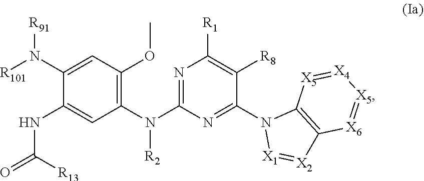

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

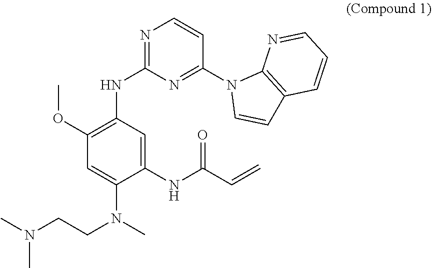 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound of Formula I is Compound 2:

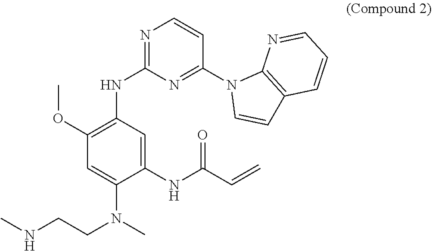 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain embodiments, the use is for treating cancer.

In certain embodiments, the use is for treating a cancer is selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas and myelomas.

In certain embodiments, the use is for treating glioblastoma.

In certain embodiments, the use is for treating lung cancer.

In certain embodiments, the use is for treating non-small cell lung cancer NSCLC.

In still other aspects, the present disclosure provides use of a compound or a pharmaceutical composition thereof for treating or reducing a brain disease or condition mediated by EGFR, comprising administering to the subject in need thereof a therapeutically effective amount of a compound having the structure of Formula I:

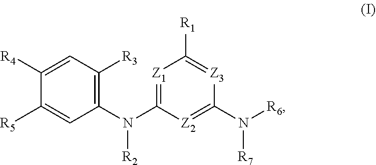

or a pharmaceutically acceptable form or an isotope derivative thereof, wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

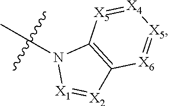

wherein
X₃ is N;
X₁, X₂, X₄, X₅ and X₆ are each independently CH or CR₁₅; and
each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CRS.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

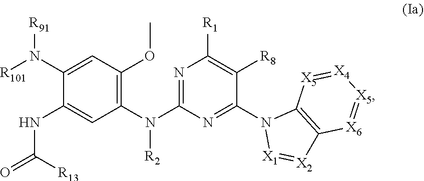

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂; or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

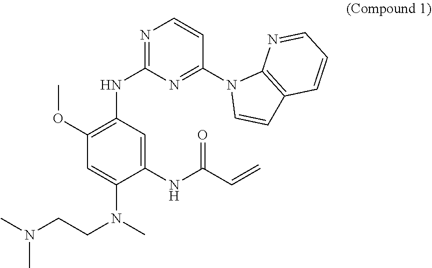 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound of Formula I is Compound 2:

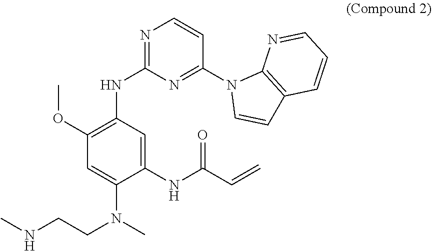 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain embodiments, the use is for treating cancer.

In certain embodiments, the use is for treating a brain cancer.

In certain embodiments, the use is for treating glioblastoma.

In still other aspects, the present disclosure provides use of a compound or a pharmaceutical composition thereof for treating or reducing a disease or condition, wherein the compound has the structure of Formula I:

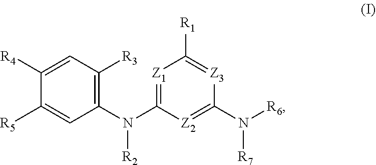

or a pharmaceutically acceptable form or an isotope derivative thereof, wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

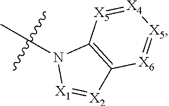

wherein
X₃ is N;
X₁, X₂, X₄, X₅ and X₆ are each independently CH or CR₁₅; and
each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CRS.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

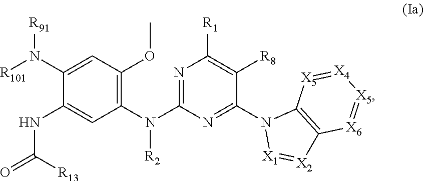

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

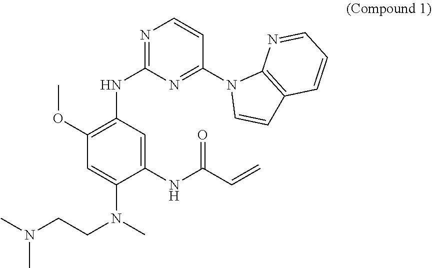 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound of Formula I is Compound 2:

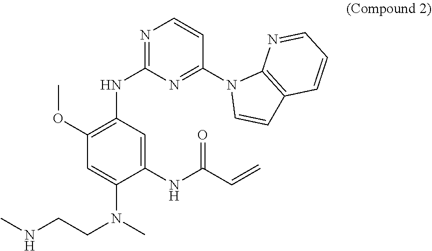 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain embodiments, the use is for treating cancer.

In certain embodiments, the use is for treating a cancer is selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas and myelomas.

In certain embodiments, the use is for treating glioblastoma.

In certain embodiments, the use is for treating lung cancer.

In certain embodiments, the use is for treating non-small cell lung cancer NSCLC.

In still other aspects, the present disclosure provides use of a compound or a pharmaceutical composition thereof for inhibiting or reducing the activity of EGFR in a subject suffering from a disease or condition related thereto, wherein the compound has the structure of Formula I:

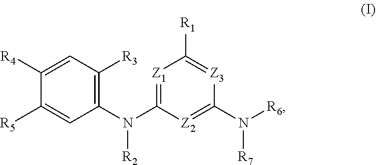

or a pharmaceutically acceptable form or an isotope derivative thereof, wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

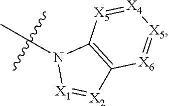

wherein
X₃ is N;
X₁, X₂, X₄, X₅ and X₆ are each independently CH or CR₁₅; and
each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CRS.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

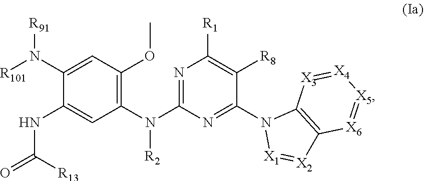

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂; or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

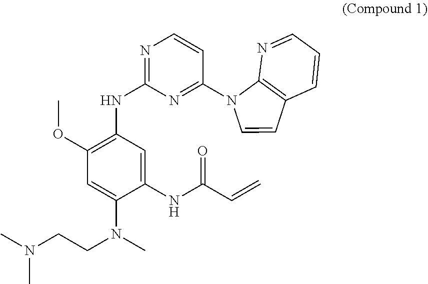 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound of Formula I is Compound 2:

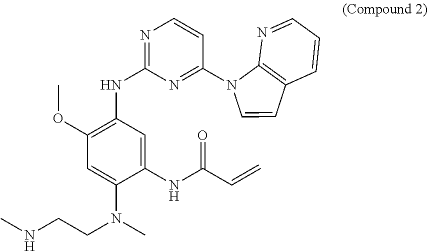 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain embodiments, the use is for treating cancer.

In certain embodiments, the use is for treating a cancer is selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas and myelomas.

In certain embodiments, the use is for treating glioblastoma.

In certain embodiments, the use is for treating lung cancer.

In certain embodiments, the use is for treating non-small cell lung cancer NSCLC.

In still other aspects, the present disclosure provides use of a compound or a pharmaceutical composition thereof for treating or reducing a disease or condition mediated by EGFR, wherein the compound has the structure of Formula I:

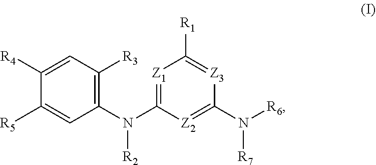

or a pharmaceutically acceptable form or an isotope derivative thereof, wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula,

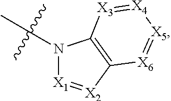

wherein
X₃ is N;
X₁, X₂, X₄, X₅ and X₆ are each independently CH or CR₁₅; and
each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

In certain embodiments, Z₁ and Z₂ are each N and Z₃ is CRS.

In certain embodiments, R₁ is H or NH₂, such as H.

In certain embodiments, R₃ is (C₁-C₄) alkoxy.

In certain embodiments, R₄ is NR₉R₁₀.

In certain embodiments, R₅ is NR₁₂C(O)R₁₃.

In certain embodiments, R₁₅ is selected from (C₁-C₆) alkyl and (C₁-C₆) haloalkyl. In some such embodiments, R₁₅ is selected from methyl and CF₃.

In certain embodiments, R₈ is H or halogen.

In certain embodiments, R₉ is (C₁-C₄) alkyl.

In certain embodiments, R₁₀ is (C₁-C₄) alkyl-NH(C₁-C₄) alkyl, or (C₁-C₄) alkyl-N((C₁-C₄) alkyl)₂.

In certain embodiments, R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

In certain embodiments, R₁₁ is (C₁-C₄) alkyl, R₁₂ is H, and R₁₃ is (C₂-C₆) alkenyl. In other embodiments, R₁₁ is (C₁-C₄) alkyl, R₁₂ is (C₁-C₆) alkyl, and R₁₃ is (C₂-C₆) alkenyl.

In some embodiments, the compound of Formula I is a compound of Formula Ia:

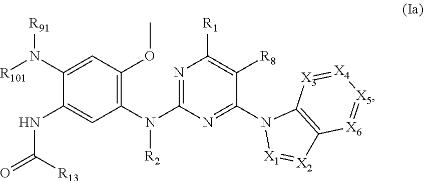

or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5- to 7-membered heterocycle optionally comprising 1 or 2 additional heteroatoms selected from N, O, and S and optionally substituted with one or more R₁₁.

In certain embodiments, the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
   or a pharmaceutically acceptable salt thereof.

In certain embodiments, the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

In certain embodiments, the compound of Formula I is Compound 1:

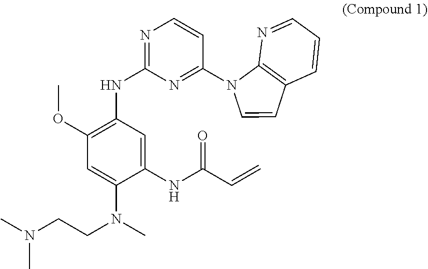 [SMILES: C=CC(=O)Nc1cc(Nc2nccc(-n3ccc4cccnc43)n2)c(OC)cc1N(C)CCN(C)C]

or a pharmaceutically acceptable form or an isotope derivative thereof.

In certain embodiments, the compound of Formula I is Compound 2:

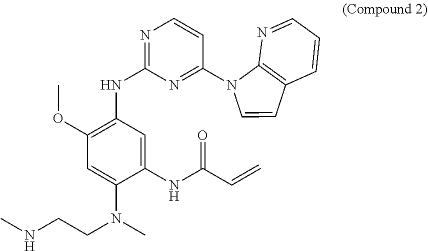 [SMILES: C=CC(=O)Nc1cc(Nc2nccc(-n3ccc4cccnc43)n2)c(OC)cc1N(C)CCNC]

or a pharmaceutically acceptable form or an isotope derivative thereof.

In certain embodiments, the use is for treating cancer.

In certain embodiments, the use is for treating a cancer is selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas and myelomas.

In certain embodiments, the use is for treating glioblastoma.

In certain embodiments, the use is for treating lung cancer.

In certain embodiments, the use is for treating non-small cell lung cancer NSCLC.

### Definitions

Listed below are definitions of various terms used to describe this application.

These definitions apply to the terms as they are used throughout this specification and claims, unless otherwise limited in specific instances, either individually or as part of a larger group.

As used herein, “at least” a specific value is understood to be that value and all values greater than that value.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term “about.”

As used herein, the term “administration” of a disclosed compound encompasses the delivery to a subject of a compound as described herein, or a prodrug or other pharmaceutically acceptable form thereof, using any suitable formulation or route of administration, as discussed herein.

The terms “disease”, “disorder” and “condition” are used interchangeably unless indicated otherwise.

The terms “cancer” or “tumor” are used interchangeably herein and refer to diseases or disorders involving abnormal cell growth and/or proliferation, such as glioma, thyroid carcinoma, breast carcinoma, brain cancer (e.g., glioblastoma), lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer).

As used herein, the terms “effective amount” or “therapeutically effective amount” refer to that amount of a compound or pharmaceutical composition described herein that is sufficient to effect the intended application including, but not limited to, disease treatment, as illustrated below.

In some embodiments, the amount is that effective for detectable killing or inhibition of the growth or spread of cancer cells; the size or number of tumors; or other measure of the level, stage, progression or severity of the cancer.

The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The term also applies to a dose that will induce a particular response in target cells, e.g., reduction of cell migration. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

The term “alkyl,” as used herein, refers to saturated, straight- or branched-chain hydrocarbon radicals containing, in certain embodiments, between one and six, or one and eight carbon atoms, respectively. Examples of C₁-C₆ alkyl radicals include, but are not limited to, methyl, ethyl, propyl, isopropyl, n-butyl, tert-butyl, neopentyl, n-hexyl radicals; and examples of C₁-C₈ alkyl radicals include, but are not limited to, methyl, ethyl, propyl, isopropyl, n-butyl, tert-butyl, neopentyl, n-hexyl, heptyl, octyl radicals.

The term “alkenyl,” as used herein, denotes a monovalent group derived from a hydrocarbon moiety containing, in certain embodiments, from two to six, or two to eight carbon atoms having at least one carbon-carbon double bond. The double bond may or may not be the point of attachment to another group. Alkenyl groups include, but are not limited to, for example, ethenyl, propenyl, butenyl, 1-methyl-2-buten-1-yl, heptenyl, octenyl and the like.

The term “alkynyl,” as used herein, denotes a monovalent group derived from a hydrocarbon moiety containing, in certain embodiments, from two to six, or two to eight carbon atoms having at least one carbon-carbon triple bond. The alkynyl group may or may not be the point of attachment to another group. Representative alkynyl groups include, but are not limited to, for example, ethynyl, 1-propynyl, 1-butynyl, heptynyl, octynyl and the like.

The term “alkoxy” refers to an —O-alkyl radical.

The term “aryl,” as used herein, refers to a mono- or poly-cyclic carbocyclic ring system having one or more aromatic rings, fused or non-fused, including, but not limited to, phenyl, naphthyl, tetrahydronaphthyl, indanyl, indenyl and the like.

The term “aralkyl,” as used herein, refers to an alkyl residue attached to an aryl ring. Examples include, but are not limited to, benzyl, phenethyl and the like.

The term “cycloalkyl,” as used herein, denotes a monovalent group derived from a monocyclic or polycyclic saturated or partially unsaturated carbocyclic ring compound.

Examples of C₃-C₈-cycloalkyl include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo [2.2.1]heptyl, and bicyclo [2.2.2] octyl. Also contemplated is a monovalent group derived from a monocyclic or polycyclic carbocyclic ring compound having at least one carbon-carbon double bond by the removal of a single hydrogen atom. Examples of such groups include, but are not limited to, cyclopropenyl, cyclobutenyl, cyclopentenyl, cyclohexenyl, cycloheptenyl, cyclooctenyl, and the like.

The term “heteroaryl,” as used herein, refers to a mono- or poly-cyclic (e.g., bi-, or tri-cyclic or more) fused or non-fused, radical or ring system having at least one aromatic ring, having from five to ten ring atoms of which one ring atoms is selected from S, O, and N; zero, one, or two ring atoms are additional heteroatoms independently selected from S, O, and N; and the remaining ring atoms are carbon. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

The term “heteroaralkyl,” as used herein, refers to an alkyl residue attached to a heteroaryl ring. Examples include, but are not limited to, pyridinylmethyl, pyrimidinylethyl and the like.

The term “heterocyclyl,” or “heterocycloalkyl,” as used herein, refers to a non-aromatic 3-, 4-, 5-, 6- or 7-membered ring or a bi- or tri-cyclic group fused of non-fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, and (v) any of the above rings may be fused to a benzene ring. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “alkylamino” refers to a group having the structure —NH(C₁-C₁₂ alkyl) where C₁-C₁₂ alkyl is as previously defined.

The term “dialkylamino” refers to a group having the structure —N(C₁-C₁₂ alkyl)₂ where C₁-C₁₂ alkyl is as previously defined.

The term “acyl” includes residues derived from acids, including but not limited to carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates and aliphatic phosphates. Examples of aliphatic carbonyls include, but are not limited to, acetyl, propionyl, 2-fluoroacetyl, butyryl, 2-hydroxy acetyl, and the like.

In accordance with the application, any of the aryls, substituted aryls, heteroaryls and substituted heteroaryls described herein, can be any aromatic group. Aromatic groups can be substituted or unsubstituted.

The terms “hal,” “halo,” and “halogen,” as used herein, refer to an atom selected from fluorine, chlorine, bromine and iodine.

As described herein, compounds of the application may optionally be substituted with one or more substituents, such as are illustrated generally above, or as exemplified by particular classes, subclasses, and species of the application. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. The terms “optionally substituted”, “optionally substituted alkyl,” “optionally substituted “optionally substituted alkenyl,” “optionally substituted alkynyl”, “optionally substituted cycloalkyl,” “optionally substituted cycloalkenyl,” “optionally substituted aryl”, “optionally substituted heteroaryl,” “optionally substituted aralkyl”, “optionally substituted heteroaralkyl,” “optionally substituted heterocycloalkyl,” and any other optionally substituted group as used herein, refer to groups that are substituted or unsubstituted by independent replacement of one, two, or three or more of the hydrogen atoms thereon with substituents including, but not limited to: —F, —Cl, —Br, —I, —OH, protected hydroxy, —NO₂, —CN, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkenyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkenyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkenyl, —CONH—C₃-C₁₂-cycloalkyl, —CONH-aryl, —CONH-heteroaryl, —CONH-heterocycloalkyl, —OCO₂—C₁-C₁₂-alkyl; —OCO₂—C₂-C₁₂-alkenyl, —OCO₂—C₂-C₁₂-alkenyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkenyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkenyl, —NHC(O)—C₃-C₁₂-cycloalkyl, —NHC(O)-aryl, —NHC(O)— heteroaryl, —NHC(O)— heterocycloalkyl, —NHCO₂—C₁-C₁₂-alkyl, —NHCO₂—C₂-C₁₂-alkenyl, —NHCO₂—C₂-C₁₂-alkenyl, —NHCO₂—C₃-C₁₂-cycloalkyl, —NHCO₂-aryl, —NHCO₂-heteroaryl, —NHCO₂-heterocycloalkyl, NHC(O)NH₂, —NHC(O)NH—C₁-C₁₂-alkyl, —NHC(O)NH—C₂-C₁₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, NHC(O)NH— heterocycloalkyl, NHC(S)NH₂, —NHC(S)NH—C₁-C₁₂-alkyl, —NHC(S)NH—C₂-C₁₂-alkenyl, —NHC(S)NH—C₂-C₁₂-alkenyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₁₂-alkenyl, —NHC(NH)NH—C₂-C₁₂-alkenyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₁₂-alkenyl, —NHC(NH)—C₂-C₁₂-alkenyl, —NHC(NH)—C₃-C₁₂-cycloalkyl, —NHC(NH)-aryl, —NHC(NH)-heteroaryl, —NHC(NH)— heterocycloalkyl, —C(NH)NH—C₁-C₁₂-alkyl, —C(NH)NH—C₂-C₁₂-alkenyl, —C(NH)NH—C₂-C₁₂-alkenyl, C(NH)NH—C₃-C₁₂-cycloalkyl, —C(NH)NH-aryl, —C(NH)NH-heteroaryl, —C(NH)NHheterocycloalkyl, —S(O)—C₁-C₁₂-alkyl, —S(O)—C₂-C₁₂-alkenyl, —S(O)—C₂-C₁₂-alkenyl, —S(O)—C₃-C₁₂-cycloalkyl, —S(O)-aryl, —S(O)-heteroaryl, —S(O)-heterocycloalkyl-SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkenyl, —S—C₃-C₁₂-cycloalkyl, —S-aryl, —S— heteroaryl, —S-heterocycloalkyl, or methylthiomethyl.

It is understood that the aryls, heteroaryls, alkyls, and the like can be further substituted.

The term “EGFR” herein refers to epidermal growth factor receptor kinase.

The term “HER” or “Her”, herein refers to human epidermal growth factor receptor kinase.

The term “subject” as used herein refers to any animal (e.g., a mammal), including, but not limited to, humans, non-human primates, rodents, and the like, which is to be the recipient of a particular treatment. A subject therefore refers to, for example, dogs, cats, horses, cows, pigs, guinea pigs, and the like. Preferably the subject is a human. When the subject is a human, the subject may be referred to herein as a patient. In some embodiments, the subject has an EGFR mutation. In other embodiments, the subject has T790M EGFR mutation. In other embodiments, the subject has deletion in exon 19 EGFR mutation. In some embodiments, the subject has L858R/T790M EGFR mutation.

“Treat”, “treating” and “treatment” refer to a method of alleviating or abating a disease and/or its attendant symptoms.

As used herein, a “pharmaceutically acceptable form” of a disclosed compound includes, but is not limited to, pharmaceutically acceptable salts, esters, hydrates, solvates, isomers, prodrugs, and isotopically labeled derivatives of disclosed compounds. In one embodiment, a “pharmaceutically acceptable form” includes, but is not limited to, pharmaceutically acceptable salts, esters, isomers, prodrugs and isotopically labeled derivatives of disclosed compounds. In some embodiments, a “pharmaceutically acceptable form” includes, but is not limited to, pharmaceutically acceptable salts, esters, stereoisomers, prodrugs and isotopically labeled derivatives of disclosed compounds.

As used herein, the term “pharmaceutically acceptable salt” refers to those salts of the compounds formed by the process of the present application which are, within the scope of sound medical judgment, suitable for use in contact with the tissues of humans and lower animals without undue toxicity, irritation, allergic response and the like, and are commensurate with a reasonable benefit/risk ratio; salts that are not pharmaceutically acceptable may, however, be useful in the preparation of the compounds described herein of their pharmaceutically acceptable salts. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the application, or separately, such as by reacting the free base function with a suitable organic acid. When a compound is acidic, suitable “pharmaceutically acceptable salts” refers to salts prepared from pharmaceutically acceptable non-toxic bases including inorganic and organic bases. Salts derived from inorganic bases include aluminum, ammonium, calcium, copper, ferric, ferrous, lithium, magnesium, manganic, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines, arginine, betaine, caffeine, choline, N, N¹-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like. When a compound is basic, salts may be prepared from pharmaceutically acceptable non-toxic acids, including inorganic and organic acids. Such acids include acetate, acetic, acid citrate, acid phosphate, ascorbate, benzenesulfonic, benzenesulfonate, benzoic, benzoate, bromide, bisulfate, bitartrate, camphorsulfonic, chloride, citrate, citric, ethanesulfonate, ethanesulfonic, formate, fumarate, fumaric, gentisinate, gluconate, gluconic, glucuronate, glutamate, glutamic, hydrobromic, hydrochloric, iodide, isethionic, isonicotinate, lactate, lactic, maleate, maleic, malic, mandelic, methanesulfonic, methanesulfonate, mucic, nitrate, nitric, oleate, oxalate, pamoic, pamoate (i.e., 1,1′-methylene-bis-(2-hydroxy-3-naphthoate)), pantothenic, pantothenate, phosphate, phosphoric, saccharate, salicylate, succinic, succinate, sulfuric, sulfate, tannate, tartrate, tartaric, p-toluenesulfonate, toluenesulfonic acid (TsOH) and the like. Particular embodiments include TsOH, citric, hydrobromic, hydrochloric, maleic, phosphoric, sulfuric and tartaric acids.

Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters of the compounds formed by the process of the present application which hydrolyze in vivo and include those that break down readily in the human body to leave the parent compound or a salt thereof. Such esters can act as a prodrug as defined herein. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfinic acids, sulfonic acids and boronic acids. Examples of particular esters include, but are not limited to, formates, acetates, propionates, butyrates, acrylates and ethylsuccinates. The esters can be formed with a hydroxy or carboxylic acid group of the parent compound.

The term “pharmaceutically acceptable prodrugs” as used herein refers to those prodrugs of the compounds formed by the process of the present application which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present application. “Prodrug”, as used herein means a compound which is convertible in vivo by metabolic means (e.g., by hydrolysis) to afford any compound delineated by the formulae of the instant application. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al. (ed). “Design and Application of Prodrugs, Textbook of Drug Design and Development, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002). A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

This application also encompasses pharmaceutical compositions containing, and methods of treating disorders through administering, pharmaceutically acceptable prodrugs of compounds of the application. For example, compounds of the application having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the application. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxy carbonyls, as outlined in Advanced Drug Delivery Reviews, 1996, 19, 1 15. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities

Combinations of substituents and variables envisioned by this application are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject, formulation into therapeutic products, intermediates for use in production of therapeutic compounds, isolatable or storable intermediate compounds, treating a disease or condition responsive to therapeutic agents).

The application also provides for methods using a pharmaceutical composition comprising a compound of Formula (I), or a pharmaceutically acceptable ester, salt, or prodrug thereof, together with a pharmaceutically acceptable carrier.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

Suitable carriers, diluents and excipients well known to those skilled in the art include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which a compound described herein is being formulated. Solvents are generally selected based on solvents recognized by persons skilled in the art as safe (GRAS-Generally Regarded as Safe) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g., enteric or slow release) preservatives, antioxidants, opaquing agents, glidants, processing aids, colorants, sweeteners, perfuming agents, flavoring agents and other known additives to provide an elegant presentation of the drug (i.e., a compound described herein or pharmaceutical composition thereof) or aid in the manufacturing of the pharmaceutical product (i.e., medicament).

In another aspect, the application provides methods using a kit comprising a compound capable of inhibiting EGFR activity selected from one or more compounds of Formula (I), or a pharmaceutically acceptable salt, hydrate, solvate, prodrug, stereoisomer, or tautomer thereof, and instructions for use in treating cancer.

Compounds of Formula (I) and methods of synthesizing them are described in U.S. Pat. No. 10,266,517, which is hereby incorporated by reference in its entirety.

Another aspect is an isotopically labeled compound of any of the formulae delineated herein. Such compounds have one or more isotope atoms which may or may not be radioactive (e.g., ³H, ²H, ¹⁴C, ¹³C, ¹⁸, ³⁵S, ³²P, ¹²⁵I, and ¹³¹I) introduced into the compound. Such compounds are useful for drug metabolism studies and diagnostics, as well as therapeutic applications.

The present disclosure encompasses salts of the compounds disclosed herein and their pharmaceutical compositions. A salt of a compound of the present disclosure may be formed between an acid and a basic group of the compound, such as an amino functional group, or a base and an acidic group of the compound, such as a carboxyl functional group. According to other embodiments, the compound is a pharmaceutically acceptable acid addition salt. A compound of the application can be prepared as a pharmaceutically acceptable acid addition salt by reacting the free base form of the compound with a pharmaceutically acceptable inorganic or organic acid. Alternatively, a pharmaceutically acceptable base addition salt of a compound of the application can be prepared by reacting the free acid form of the compound with a pharmaceutically acceptable inorganic or organic base.

Alternatively, the salt forms of the compounds of the application can be prepared using salts of the starting materials or intermediates.

The free acid or free base forms of the compounds of the application can be prepared from the corresponding base addition salt or acid addition salt from, respectively. For example a compound of the application in an acid addition salt form can be converted to the corresponding free base by treating with a suitable base (e.g., ammonium hydroxide solution, sodium hydroxide, and the like). A compound of the application in a base addition salt form can be converted to the corresponding free acid by treating with a suitable acid (e.g., hydrochloric acid, etc.).

Prodrug derivatives of the compounds of the application can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the application with a suitable carbamylating agent (e.g., 1,1-acyloxyalkylcarbanochloridate, para-nitrophenyl carbonate, or the like).

The compounds of the present invention may be prepared by methods known in the art of organic synthesis as set forth in part by the following synthetic schemes. In the schemes described below, it is well understood that protecting groups for sensitive or reactive groups are employed where necessary in accordance with general principles of chemistry. Protected derivatives of the compounds of the application can be made by means known to those of ordinary skill in the art. A detailed description of techniques applicable to the creation of protecting groups and their removal can be found in T. W. Greene, “Protecting Groups in Organic Chemistry”, 3rd edition, John Wiley and Sons, Inc., 1999. These groups may be removed at a convenient stage of the compound synthesis using methods that are readily apparent to those skilled in the art. The selection processes, as well as the reaction conditions and order of their execution, shall be consistent with the preparation of the compounds described herein.

The compounds described herein may be made from commercially available starting materials or synthesized using known organic, inorganic, and/or enzymatic processes.

All the abbreviations used in this application are found in “Protective Groups in Organic Synthesis” by John Wiley & Sons, Inc, or the MERCK INDEX by MERCK & Co., Inc, or other chemistry books or chemicals catalogs by chemicals vendor such as Aldrich, or according to usage known in the art.

The synthesis of the compounds described herein may be readily achieved by synthetic chemists of ordinary skill by reference to the Exemplary Synthesis and Examples disclosed herein. Such methods can be carried out utilizing corresponding deuterated and optionally, other isotope-containing reagents and/or intermediates to synthesize the compounds described herein, or invoking standard synthetic protocols known in the art for introducing isotopic atoms to a chemical structure.

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can pro-duce the desired bridged macrocyclic products of the present application. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

As used herein, the term an “isolated” or “substantially isolated” molecule (such as a polypeptide or polynucleotide) is one that has been manipulated to exist in a higher concentration than in nature or has been removed from its native environment. For example, a subject antibody is isolated, purified, substantially isolated, or substantially purified when at least 10%, or 20%, or 40%, or 50%, or 70%, or 90% of non-subject-antibody materials with which it is associated in nature have been removed. For example, a polynucleotide or a polypeptide naturally present in a living animal is not “isolated,” but the same polynucleotide or polypeptide separated from the coexisting materials of its natural state is “isolated.” Further, recombinant DNA molecules contained in a vector are considered isolated for the purposes of the present invention. Isolated RNA molecules include in vivo or in vitro RNA replication products of DNA and RNA molecules. Isolated nucleic acid molecules further include synthetically produced molecules. Additionally, vector molecules contained in recombinant host cells are also isolated. Thus, not all “isolated” molecules need be “purified.”

As used herein, the term “purified” when used in reference to a molecule, it means that the concentration of the molecule being purified has been increased relative to molecules associated with it in its natural environment, or environment in which it was produced, found or synthesized. Naturally associated molecules include proteins, nucleic acids, lipids and sugars but generally do not include water, buffers, and reagents added to maintain the integrity or facilitate the purification of the molecule being purified. According to this definition, a substance may be 5% or more, 10% or more, 20% or more, 30% or more, 40% or more, 50% or more, 60% or more, 70% or more, 80% or more, 90% or more, 95% or more, 98% or more, 99% or more, or 100% pure when considered relative to its contaminants.

Some aspects of the present invention include a method of inhibiting the activity of EGFR in a subject comprising administering to the subject an effective amount of at least one compound described herein, or a pharmaceutically acceptable salt thereof, or a pharmaceutical composition described herein.

In some embodiments, the compound described herein, or a pharmaceutically acceptable salt thereof, or a pharmaceutical composition described herein is capable of inhibiting the activity of EGFR containing one or more mutations. In some embodiments, the mutant EGFR contains one or more mutations selected from T790M, L718Q, L844Y, L858R, and Del. In some embodiments, the mutant EGFR contains a combination of mutations, wherein the combination is selected from Del/L718Q, Del/L844Y, Del/T790M, Del/T790M/L718Q, Del/T790M/L844Y, L858R/L718Q, L858R/L844Y, L858R/T790M, and L858R/T790M/L718Q. In some embodiments, the EGFR mutation is T790M mutation. In other embodiments, the EGFR mutation is deletion in exon 19. In particular embodiments, the EGFR mutation is L858R/T790M mutation.

In some embodiments, the compound described herein, or a pharmaceutically acceptable salt thereof, or a pharmaceutical composition described herein is capable of inhibiting the activity of EGFR containing one or more mutations, but do not affect the activity of a wild-type EGFR.

Inhibition of EGFR containing one or more mutations, such as those described herein, but not a wild-type EGFR, provides a novel approach to the treatment, prevention, or amelioration of diseases including, but not limited to, cancer and metastasis, inflammation, arthritis, systemic lupus erythematous, skin-related disorders, pulmonary disorders, cardiovascular disease, ischemia, neurodegenerative disorders, liver disease, gastrointestinal disorders, viral and bacterial infections, central nervous system disorders, Alzheimer's disease, Parkinson's disease, Huntington's disease, amyotrophic lateral sclerosis, spinal cord injury, and peripheral neuropathy.

In some embodiments, a drug-resistant EGFR mutant comprises a sensitizing mutation, such as Del and L858R.

In some embodiments, the application provides a compound inhibiting kinase activity of a drug-resistant EGFR mutant harboring a sensitizing mutation (e.g., Del and L858R) and a drug-resistance mutation (e.g., T790M, L718Q, and L844V) with less than a 10-fold difference in potency (e.g., as measured by IC50) relative to an EGFR mutant harboring the sensitizing mutation but not the drug-resistance mutation. In some embodiments, the difference in potency is less than about 9-fold, 8-fold, 7-fold, 6-fold, 5-fold, 4-fold, 3-fold, or 2-fold.

In some embodiments, the present disclosure provides a compound that is more potent than one or more known EGFR inhibitors, including but not limited to gefitinib, erlotinib, lapatinib, WZ4002, HKI-272, CL-387, 785, and AZD9291, at inhibiting the activity of EGFR containing one or more mutations as described herein, such as T790M, L718Q, L844Y, L858R, Del, or a combination thereof. For example, the compound can be at least about 2-fold, 3-fold, 5-fold, 10-fold, 25-fold, 50-fold or about 100-fold more potent (e.g., as measured by IC50) than gefitinib, erlotinib, lapatinib, WZ4002, HKI-272, CL-387, 785, and AZD9291 at inhibiting the activity of the EGFR containing one or more mutations as described herein. In other embodiments, the application provides a compound that is less potent than one or more known EGFR inhibitors, including but not limited to gefitinib, erlotinib, lapatinib, WZ4002, HKI-272, CL-387, 785, and AZD9291, at inhibiting the activity of EGFR containing one or more mutations as described herein, such as T790M, L718Q, L844Y, L858R, Del, or a combination thereof.

Potency of a compound can be determined by IC50 value. A compound with a lower IC50 value, as determined under substantially similar conditions, is a more potent inhibitor relative to a compound with a higher IC50 value. In some embodiments, the substantially similar conditions comprise determining an EGFR-dependent phosphorylation level in 3T3 cells expressing a wild type EGFR, a mutant EGFR, or a fragment of any thereof.

An EGFR sensitizing mutation comprises without limitation L858R, G719S, G719C, G719A, L861Q, a deletion in exon 19 and/or an insertion in exon 20. Drug-resistant EGFR mutants can have without limitation a drug resistance mutation comprising T790M, T854A, L718Q or D761Y.

An alternative method to measure effects on EGFR activity is to assay EGFR phosphorylation. Wild type or mutant (L858R/T790M, Del/T790M, Del/T790M/L 718Q, or L858R/T790M/L718Q) EGFR can be transfected into NIH-3T3 cells (which do not normally express endogenous EGFR) and the ability of the inhibitor (using concentrations as above) to inhibit EGFR phosphorylation can be assayed. Cells are exposed to increasing concentrations of inhibitor for 6 hours and stimulated with EGFR for 10 minutes. The effects on EGFR phosphorylation are assayed by Western Blotting using phospho-specific (Y1068) EGFR antibodies.

In some embodiments, the present invention provides methods of treating a disease mediated by EGFR in a subject comprising administering to the subject an effective amount of at least one compound described herein, or a pharmaceutically acceptable salt thereof, or a pharmaceutical composition described herein. In some embodiments of the above disclosed aspect, the disease mediated by EGFR is cancer.

In some embodiments, the present invention provides a method of treating lung cancer. In some embodiments, the present invention provides a method of treating non-small cell lung cancer (NSCLC). In some embodiments, the present invention provides a method of treating small cell lung cancer (SCLC).

Administering a compound, or a pharmaceutically acceptable salt thereof, or a pharmaceutical composition described herein to a mammal comprises any suitable delivery method. Most suitable means of administration for a particular patient will depend on the nature and severity of the disease or condition being treated or the nature of the therapy being used and on the nature of the active compound. Administering a compound, or a pharmaceutically acceptable form (e.g., salt) thereof, or a pharmaceutical composition described herein to a mammal includes administering a compound, or a pharmaceutically acceptable form (e.g., salt) thereof, or a pharmaceutical composition described herein topically, enterally, parenterally, transdermally, transmucosally, via inhalation, intracisternally, epidurally, intravaginally, intravenously, intramuscularly, subcutaneously, intradermally or intravitreally to the mammal. Administering a compound, or a pharmaceutically acceptable salt thereof, or a pharmaceutical composition described herein to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound, or a pharmaceutically acceptable salt thereof, or a pharmaceutical composition that metabolizes within or on a surface of the body of the mammal to a compound, or a pharmaceutically acceptable salt thereof, or a pharmaceutical composition described herein.

Thus, a compound, or a pharmaceutically acceptable salt thereof, or a pharmaceutical composition described herein, may be systemically administered, e.g., orally, in combination with a pharmaceutically acceptable vehicle such as an inert diluent or an assimilable edible carrier. They may be enclosed in hard- or soft-shell gelatin capsules, may be compressed into tablets, or may be incorporated directly with the food of the patient's diet. For oral therapeutic administration, a compound, or a pharmaceutically acceptable salt thereof, or a pharmaceutical composition as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

Compositions for parenteral injection comprise pharmaceutically-acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions can also contain adjuvants such as preservative, wetting agents, emulsifying agents, and dispersing agents. Prevention of the action of microorganisms may be ensured by the inclusion of various antibacterial and antifungal agents, for example, paragen, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33 et seq.

Useful dosages of a compound described herein can be determined by comparing their in vitro activity and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound described herein, required for use in treatment can vary not only with the particular salt selected but also with the route of administration, the nature of the condition being treated and the age and condition of the patient and can be ultimately at the discretion of the attendant physician or clinician. In general, total daily dose of the compositions of the invention to be administered to a human or other mammal host in single or divided doses may be in amounts, for example, from about 0.1 to about 20 mg/kg body weight daily, from about 0.5 to about 5 mg/kg body weight, from about 5 to about 10 mg/kg body weight. In some embodiments, a dose of 5 mg/kg or less can be suitable. The desired dose may conveniently be presented in a single dose or as divided doses administered at appropriate intervals. The compound described herein can be conveniently administered in unit dosage form; for example, containing about 25 mg to about 500 mg, about 50 mg to about 300 mg, or about 100 mg to about 250 mg of active ingredient per unit dosage form.

Solid dosage forms for oral administration include capsules, tablets, pills, powders, and granules. In such solid dosage forms, the compounds described herein or derivatives thereof are admixed with at least one inert customary excipient (or carrier) such as sodium citrate or dicalcium phosphate or (i) fillers or extenders, as for example, starches, lactose, sucrose, glucose, mannitol, and silicic acid, (ii) binders, as for example, carboxymethylcellulose, alginates, gelatin, polyvinylpyrrolidone, sucrose, and acacia, (iii) humectants, as for example, glycerol, (iv) disintegrating agents, as for example, agar-agar, calcium carbonate, potato or tapioca starch, alginic acid, certain complex silicates, and sodium carbonate, (v) solution retarders, as for example, paraffin, (vi) absorption accelerators, as for example, quaternary ammonium compounds, (vii) wetting agents, as for example, cetyl alcohol, and glycerol monostearate, (viii) adsorbents, as for example, kaolin and bentonite, and (ix) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage. Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

The disclosed methods can include a kit comprising a compound, or a pharmaceutically acceptable salt thereof, or a pharmaceutical composition described herein and instructional material which can describe administering a compound, or a pharmaceutically acceptable salt thereof, or a composition described herein to a cell or a subject. This should be construed to include other embodiments of kits that are known to those skilled in the art, such as a kit comprising a (such as sterile) solvent for dissolving or suspending a compound, or a pharmaceutically acceptable salt thereof, or a composition described herein prior to administering a compound, or a pharmaceutically acceptable salt thereof, or a composition described herein to a cell or a subject. In some embodiments, the subject can be a human.

Compounds of the present application can be conveniently prepared, or formed during the process of the application, as solvates (e.g., hydrates). Hydrates of compounds of the present application can be conveniently prepared by recrystallization from an aqueous/organic solvent mixture, using organic solvents such as dioxin, tetrahydrofuran or methanol.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

In addition, in certain embodiments, some of the compounds of this application have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms, and other stereoisomeric forms that may be defined, in terms of absolute stereochemistry, as (R)- or (S)-, or as (D)- or (L)- for amino acids. All such isomeric forms of these compounds are expressly included in the present application. Optical isomers may be prepared from their respective optically active precursors by the procedures described herein, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). The compounds of this application may also be represented in multiple tautomeric forms, in such instances, the application expressly includes all tautomeric forms of the compounds described herein (e.g., alkylation of a ring system may result in alkylation at multiple sites, the application expressly includes all such reaction products). When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion. All such isomeric forms of such compounds are expressly included in the present application. All crystal forms of the compounds described herein are expressly included in the present application.

In other embodiments, the compounds or pharmaceutically acceptable salts thereof as described herein, may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

In the present specification, the structural formula of the compound represents a certain isomer for convenience in some cases, but the present application includes all isomers, such as geometrical isomers, optical isomers based on an asymmetrical carbon, stereoisomers, tautomers, and the like. In addition, a crystal polymorphism may be present for the compounds represented by the formula. It is noted that any crystal form, crystal form mixture, or anhydride or hydrate thereof is included in the scope of the present application. Furthermore, so-called metabolite which is produced by degradation of the present compound in vivo is included in the scope of the present application.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four nonidentical substituents is termed a “chiral center”.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this application include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose. Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine.

Additionally, the compounds of the present application, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc.

Solvates and polymorphs of the compounds of the invention are also contemplated herein. “Solvate” means solvent addition forms that contain either stoichiometric or non stoichiometric amounts of solvent. Some compounds have a tendency to trap a fixed molar ratio of solvent molecules in the crystalline solid state, thus forming a solvate. The solvate can be of a disclosed compound or a pharmaceutically acceptable salt thereof. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. Solvates of the compounds of the present invention include, for example, hydrates. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

The present application is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14. Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

It is to be understood that the compounds of the present application may be depicted as different tautomers. It should also be understood that when compounds have tautomeric forms, all tautomeric forms are intended to be included in the scope of the present application, and the naming of the compounds does not exclude any tautomer form.

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired bridged macrocyclic products of the present application. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this application may be modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

The compounds of the application are defined herein by their chemical structures and/or chemical names. Where a compound is referred to by both a chemical structure and a chemical name, and the chemical structure and chemical name conflict, the chemical structure is determinative of the compound's identity.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

### EXAMPLES

In order that the invention described herein may be more fully understood, the following examples are set forth. The examples described in this application are offered to illustrate the compounds, compositions, materials, device, and methods provided herein and are not to be construed in any way as limiting their scope.

Various aspects regarding synthesis, characterization and formulation for N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide (Compound 1) can be found, for example, in Gray et al., US Patent Application Publication No.: 2017/0362204 A1.

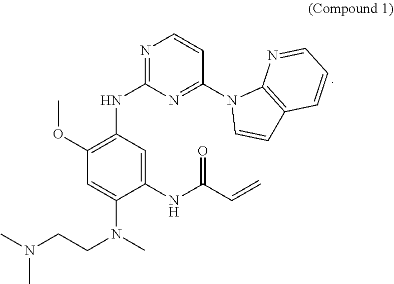 [SMILES: C=CC(=O)Nc1cc(Nc2nccc(-n3ccc4cccnc43)n2)c(OC)cc1N(C)CCN(C)C]

### Materials and Methods

### Brain Exposure

Comparative assessment of brain exposure of Compound 1 and gefitinib following single Oral (PO) or intravenous (IV) administration to sprague dawley rats were performed by Inotiv. Protocols are available upon request.

### Cell Culture

HEK293 cells and U251 cells were maintained in DMEM supplemented with 10% fetal bovine serum (FBS) and 100 μg/ml penicillin-streptomycin. Mouse neural stem cells (NSCs) were expanded in NeuroCult proliferation medium (mouse) (StemCell Technologies) supplemented with 20 ng/ml EGF. Primary mouse glioma cells (CPEvIII) were cultured in NeuroCult proliferation medium (mouse) (StemCell Technologies) supplemented with 20 ng/ml EGF, 10 ng/ml FGF and 0.0002% Heparin. Primary human glioblastoma lines BT112, BT179 and BT333 were maintained in NeuroCult proliferation medium (human) (StemCell Technologies) with 20 ng/ml EGF, 10 ng/ml FGF and 0.0002% Heparin.

### Compounds and Reagents

Gefitinib and erlotinib were purchased from Selleck Chemicals. Lapatinib was purchased from MedChemexpress. Osimertinib (AZD9291) was obtained from commercial sources. Compound 1 was synthesized by Pharmaron (Wang et al., bioRxiv, 2020.2003.2009.984500). For in vitro studies, compounds were dissolved in DMSO. For in vivo studies, Compound 1 was dissolved in 10% NMP/90% PEG300 and administered by oral gavage at 37.5 mg/kg or 75 mg/kg daily.

### Western Blot Analysis

Western blot analysis was performed as described previously (Ni, J., et al. (2012). Cancer discovery 2, 425-433; Ni, J., et al. (2016). Nature medicine 22, 723-726; Ni, J., et al. (2017). Neurooncology 19, 22-30). Anti-pEGFR-1068 (#3777), anti-pEGFR-1173 (#4407), anti-EGFR (#4267), anti-pERK1/2 (#9101), anti-ERK1/2 (#9102), anti-S6RP (#2211), and anti-S6RP (#2217) antibodies were purchased from Cell Signaling Technology. Anti-a-Tubulin antibody was purchased from Sigma.

### Cell Viability Assay

Cells were seeded in 96-well plates at a density of 1,000 cells per well and treated with two-fold serial dilutions of compounds with a starting concentration of 20 μM. Cell viability was assessed after three days of treatment by CellTiter-Glo (Promega). Curve fitting analysis and IC50 value determination were performed using GraphPad Prism 8.

### Mice

Ptenf/f (from Dr. Hong Wu, UCLA) mice were backcrossed to C57BL/6 strain background for 10 generations. They were then crossed with Cdkn2a-null (Ink4a−/−; /Arf−/−) mice (Ni et al., 2017), which are on an C₅₇BL/6 background to produce Cdkn2a-null; Ptenf/f mice. ICR-SCID mice were purchased from Taconic. All animal experiments were performed in accordance with NIH animal use guidelines and protocols approved by the Dana-Farber Cancer Institute Animal Care and Use Committee (IACUC).

### Intracranial Injections of Cells

Cells (100,000 cells resuspended in 1 μl PBS) were intracranially injected into the right striatum (0 mm anterior, 2 mm lateral, and 2.5 mm ventral to bregma) of 8-10 week-old ICR-SCID mice. Animals were monitored daily for development of neurological defects.

### Primacy Mouse CPEvIII Glioma

Neural stem cells (NSCs) from E14.5 embryonic mice (Cdkn2a-null; Ptenf/f) striata were isolated and cultured as previously described (Rietze, R. L., and Reynolds, B. A. (2006). Methods in enzymology 419, 3-23). NSCs were infected twice with adenovirus expressing Cre recombinase (AdCre; MOI50) (University of Iowa) to knock out floxed Pten. Cells were then transduced with retrovirus expressing EGFRvIII (pBabe-puro-EGFRvIII) (from Dr. Charles Stiles, DFCI) and selected with 1 μg/ml puromycin. The resulting cells (Cdkn2anull; Ptennull; EGFRvIII, denominated CPEvIII) can form a glioma after grafted into the mouse brain. Tumors were then isolated and mechanically dissociated for expansion in vitro and in vivo.

### Bioluminescence Imaging

Cells were transduced with lentiviral luciferase (HIV-Luc-zsGreen, addgene #39196). Bioluminescence signals from luciferase-expressing cells in live mice were recorded 10 minutes after intraperitoneal injection of D-luciferin (80 mg/kg) (Gold Biotechnology) with IVIS Lumina III Imaging System (PerkinElmer). The signals were analyzed with Living Image Software (PerkinElmer).

### Statistical Analysis

Statistical analysis of animal survival was determined by the log-rank (Mantel-Cox) test (Prism). Data were considered statistically significant when P<0.05.

### Example 1. Compound 1 In Vitro Activity in HEK293 Cells Expressing EGFRvIII

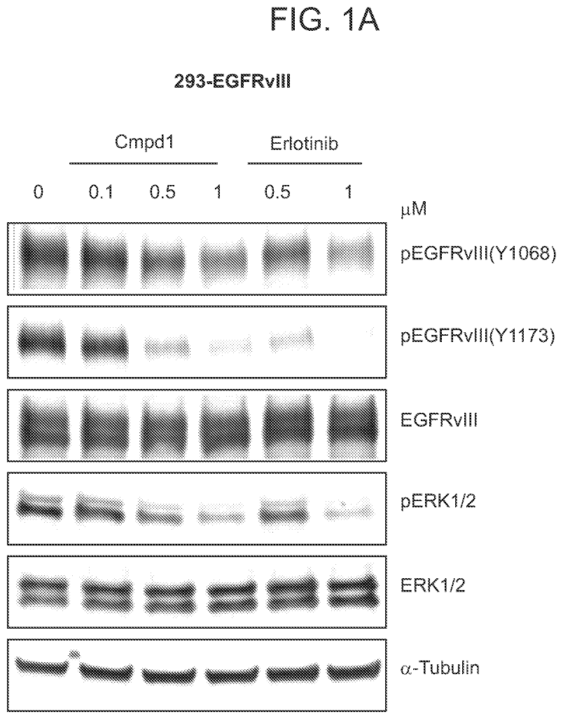

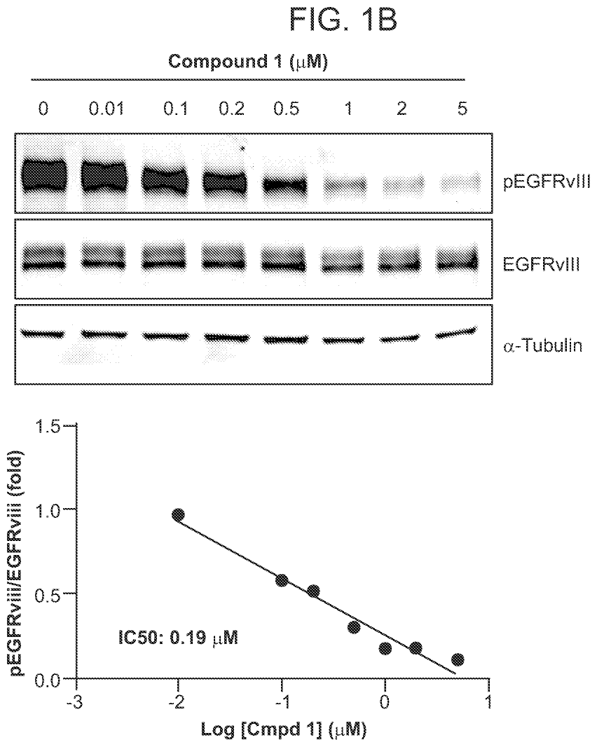

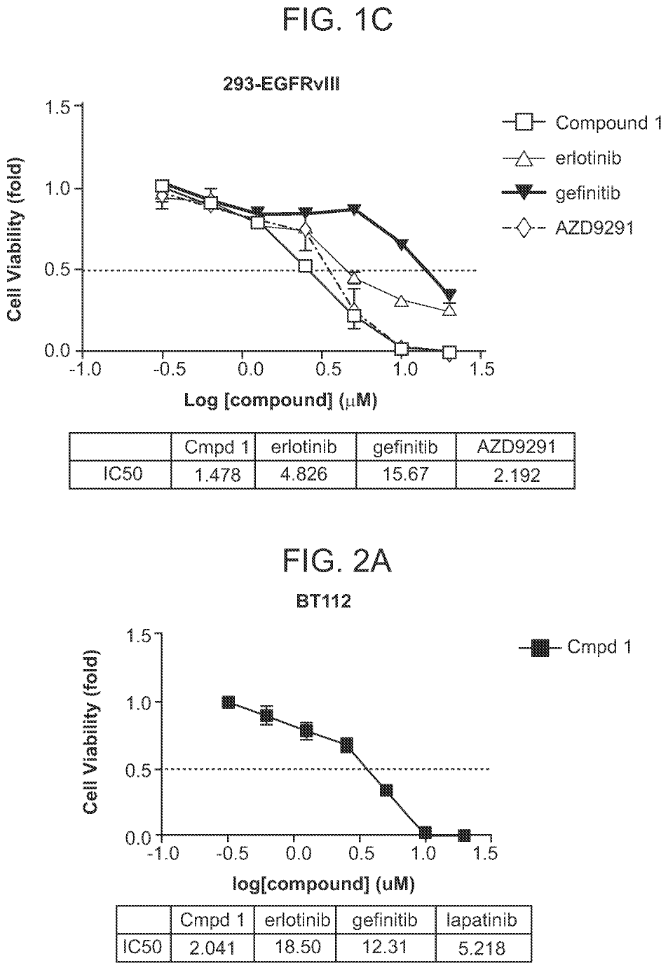

As EGFRvIII is the most common EGFR variant in GBM, the effect of Compound 1 on the activity of EGFRvIII was tested. HEK293-EGFRvIII cells stably expressing EGFRvIII (293-EGFRvIII) was generated. Compound 1 reduced the phosphorylation of EGFRvIII at both tyrosine sites 1068 and 1173 (pEGFRvIIIY1068 and pEGFRvIIIY1173), as well as phosphorylation of the downstream signaling molecules ERK1 and ERK2 (ERK1/2) in a dose-dependent manner that was comparable to that of erlotinib (FIG. 1A). Further dose titration revealed an IC50 value of 0.19 μM for Compound 1 on EGFRvIII phosphorylation (FIG. 1B). Furthermore, Compound 1 reduced viability of 293-EGFRvIII cells with an IC50 of 1.48 M, which was lower than those of erlotinib (IC50 4.83 μM), gefitinib (IC50 15.67 μM) and osimertinib (IC50 2.19 μM) (FIG. 1C).

### Example 2. Compound 1 In Vitro Activity Against GBM Patient-Derived Cell Lines Harboring EGFR Amplification and/or Mutations

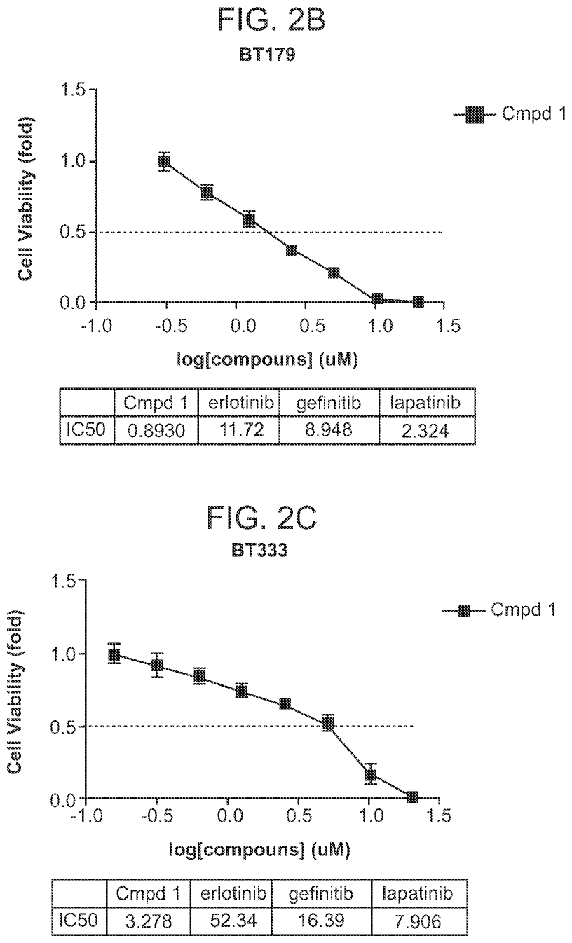

Patient-derived glioblastoma cell lines (PDCLs BT112, BT179, and BT333) characterized by EGFR amplification (EGFRamp) and/or mutation(s) were cultured and treated with Compound 1, erlotinib, gefitinib, or lapatinib (a type II EGFR TKI that is highly active against GBM EGFR variants in vitro (Vivanco, I., et al. (2012). Cancer discovery 2, 458-471)). Lapatinib more actively suppressed the survival of GBM patient-derived cells in vitro than the type I EGFR TKIs erlotinib and gefitinib (FIGS. 2A-2C). Notably, Compound 1 was the most potent TKI within this group, as shown by greater potency at reducing the viability of these PDCLs with the lowest IC50 values (Table 1).

TABLE 1
IC50 values of kinase inhibitors

| | | Compound | Erlotinib | Gefitinib | Lapatinib |
|---|---|---|---|---|---|
| | | 1 IC₅₀ | IC₅₀ | IC₅₀ | IC₅₀ |
| PDCL | EGFRamp/mutant | (μM) | (μM) | (μM) | (μM) |
| BT112 | EGFRamp/EGFRviii | 2.041 | 18.50 | 12.31 | 5.218 |
| BT179 | EGFRamp | 0.8930 | 11.72 | 8.948 | 2.324 |
| BT333 | EGFRamp/EGFR-F209I | 3.278 | 52.34 | 16.39 | 7.906 |

### Example 3. Compound 1 In Vitro Activity Against Human GBM U251 Cells Expressing EGFRvIII

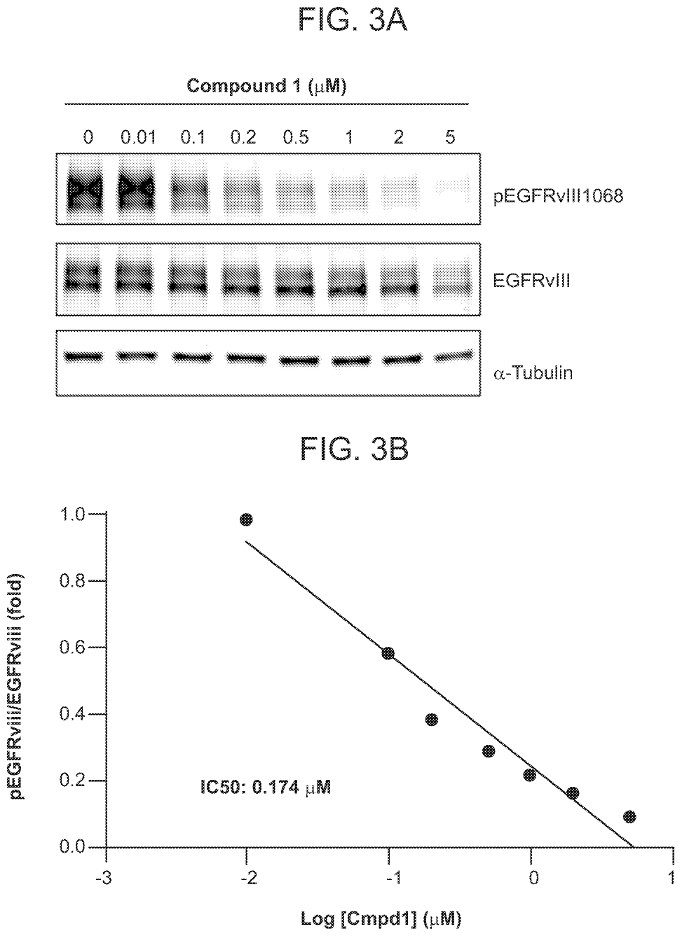

In parallel, the conventional human GBM U251 cell line was used as a surrogate model for both in vitro and in vivo studies. U251 cells were engineered to stably express EGFRvIII (U251-EGFRvIII) via retroviral-mediated gene transfer. Compound 1 was able to reduce EGFRvIII phosphorylation in U251-EGFRvIII cells in a dose-dependent manner in culture with an IC50 value of 0.174 μM (FIGS. 3A and 3B). Examination of the effect of Compound 1 along with other EGFR-TKIs on the viability of U251-EGFRvIII cells revealed that, similar to the effect on 293-EGFRvIII cells (FIGS. 1A-1C), Compound 1 and osimertinib (AZD9291 or Tagrisso®) have comparable potencies on suppressing the viability of U251-EGFRvIII cells, with IC50 values of 1.52 μM and 2.64 μM, respectively, whereas erlotinib and gefitinib showed much higher IC50 values of 13.65 μM and 20.35 μM, respectively (FIG. 3C and Table 2).

TABLE 2
IC50 values of kinase inhibitors on U251-EGFRvIII cell proliferation.

| | Compound 1 | Erlotinib | Gefitinib | Osimertinib |
|---|---|---|---|---|
| IC50 (μM) | 1.484 | 14.96 | 22.10 | 2.453 |

### Example 4. Compound 1 In Vivo Activity Against Orthotopic Xenograft Model GBM U251-EGFRvIII

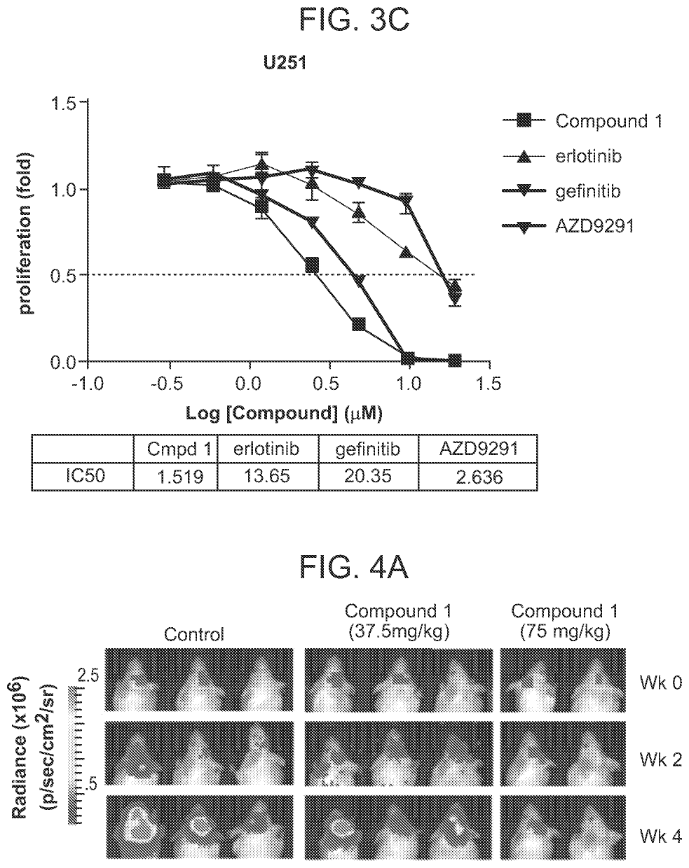

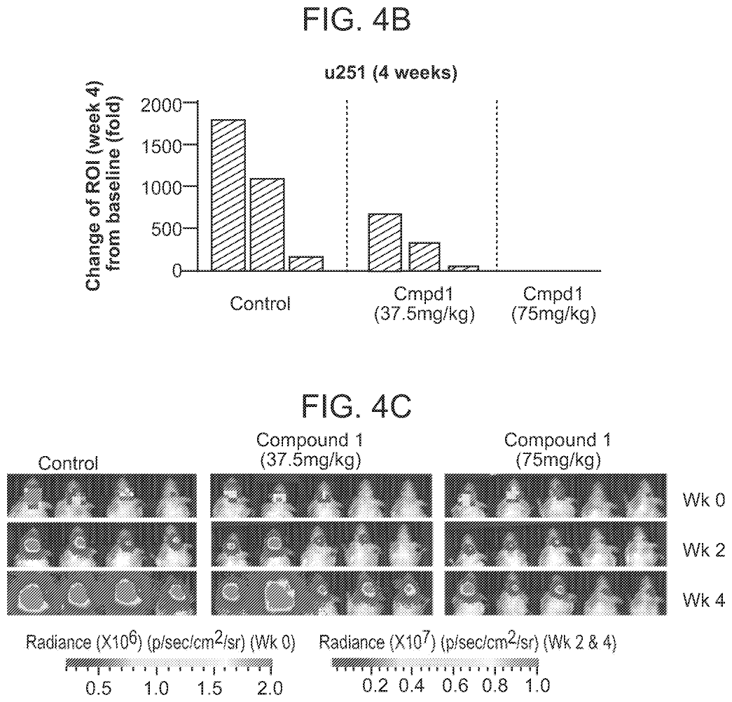

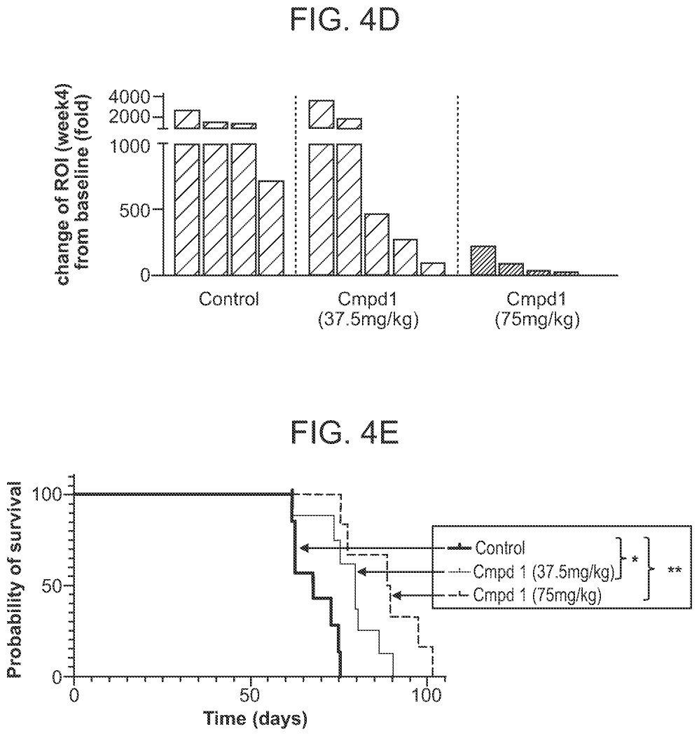

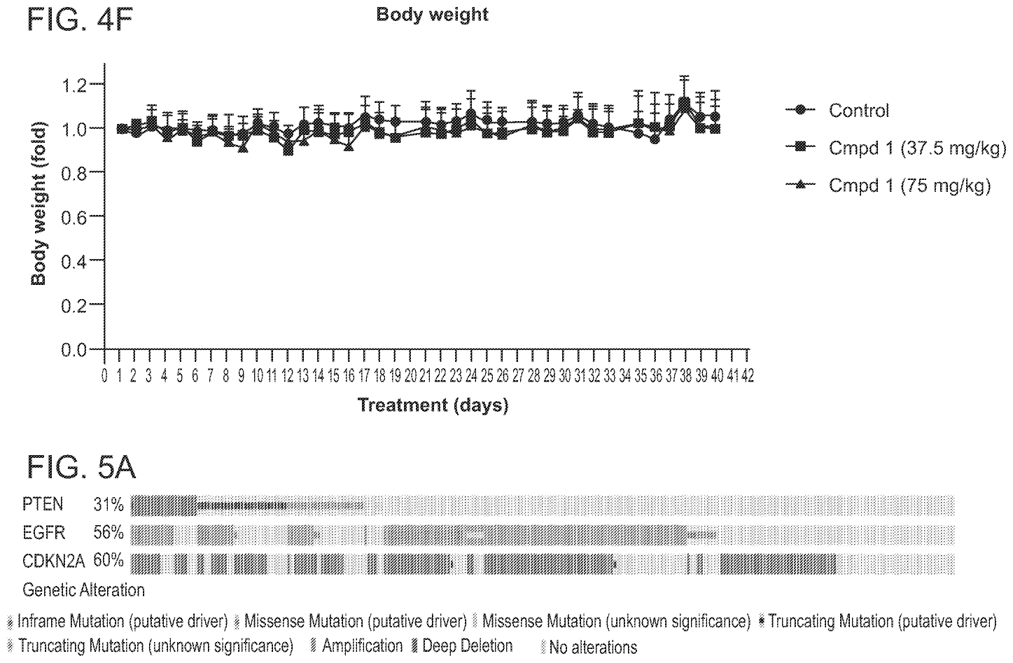

To evaluate the in vivo activity of Compound 1 in GBM with mutant EGFR, U251-EGFRvIII GBM cells expressing luciferase was generated to facilitate monitoring drug response in vivo by bioluminescence-imaging analysis. When luminescence signals were detectable, mice bearing orthotopic U251-EGFRvIII tumors were randomized into three treatment groups: vehicle control, Compound 1 at 37.5 mg/kg, or Compound 1 at 75 mg/kg. Two independent experiments were performed. Treatment with Compound 1 reduced the luminescence signals and prolonged the survival of the mice bearing orthotopic U251-EGFRvIII tumors in a dose-dependent manner in both cohorts (FIGS. 4A-4D). The medium survival was 68 days for control mice, 80 days for mice treated with Compound 1 at 37.5 mg/kg, and 89.5 days for mice treated with Compound 1 at 75 mg/kg (FIG. 4E). All mice appeared normal with no significant body weight loss during treatment time (FIG. 4F), suggesting that Compound 1 has activity in orthotopic tumors of U251-EGFRvIII and is well-tolerated in mice.

### Example 5. Compound 1 In Vitro and In Vivo Activity Against a Genetically-Engineered Mouse (GEM) Model of GBM

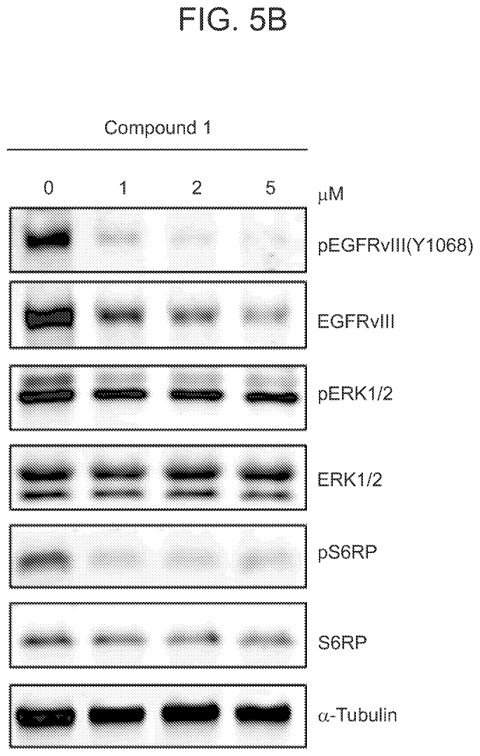

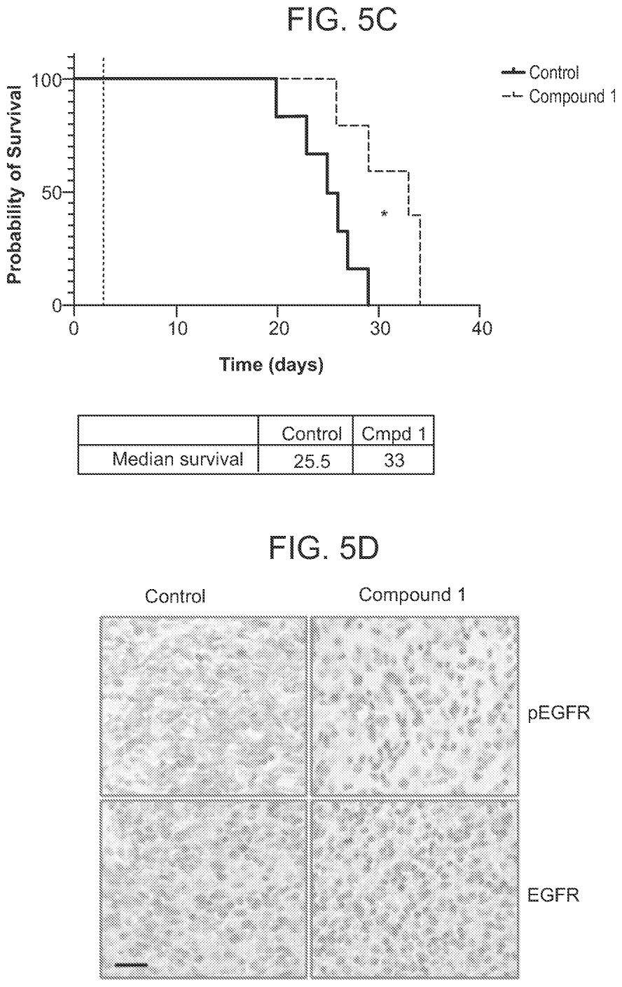

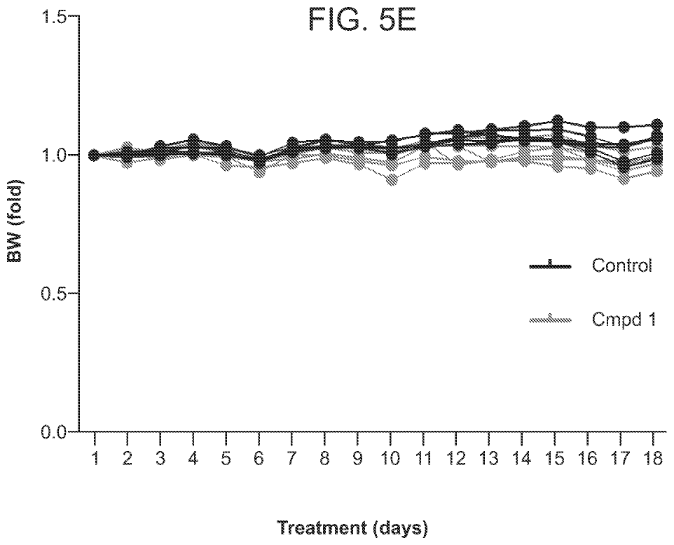

In GBM, EGFR mutations frequently coexist with CDKN2A deletion and PTEN deficiency (Brennan, C. W., et al. (2013). Cell 155, 462-477; Cancer Genome Atlas Research, N. (2008). Nature 455, 1061-1068). A syngeneic genetically engineered mouse (GEM) model of GBM driven by Cdkn2a and Pten double deletion concomitant with EGFRvIII expression (termed CPEvIII) has been generated. FIG. 5A is a diagram that shows the genetic alterations observed in these genes in GBM. Primary CPEvIII tumor cells were isolated and cultured as neurospheres and allografted intracranially in mice. As shown in FIG. 5B, Compound 1 markedly reduced phosphorylation of EGFRvIII, ERK1/2 and S6RP in a dose-dependent manner in vitro in neurosphere cultures. Compound 1 (75 mg/kg, QD) also prolonged the survival of mice bearing intracranial orthotopic allograft of CPEvIII, with medium survival of 25.5 days for control mice and 33 days for Compound 1 treated mice, p=0.017 (FIG. 5C). Immunohistochemistry (IHC) analysis of pEGFR in tumors harvested from mice at the end point revealed that phosphorylation levels of EGFRvIII were reduced in mice treated with Compound 1 (FIG. 5D). Compound 1 was also well tolerated in this cohort of mice with no significant loss of body weight observed during Compound 1 treatment (FIG. 5E). Together, these results suggest that Compound 1 has activity against CPEvIII GBM tumor cells both in vitro and in vivo.

### Example 6. Comparative Assessment of Brain Exposure of Compound 1 and Gefitinib Following Administration to Sprague Dawley Rats

Compound 1 and gefitinib were administered to Sprague Dawley rats in a single oral (PO) dose (30 mg/kg or 50 mg/kg, respectively).

Compound 1 and gefitinib were also administered to 9 week (week) male Sprague Dawley rats via intravenous infusion, Each mouse had two surgically implanted catheters for blood collection and for IV dosing (femoral and jugular, respectively). Formulations of Compound 1 and gefitinib were prepared within 24 hours of dosing. A single IV infustion for 5 hours was administered to each animal, which were then twice observed clinically (30 minutes to 1 hour post-posing and at the end of the study (T_{Terminal}). Blood samples were collected at 5 time points from each animal over the 5 hour infusion period (5 minutes, 1 hour, 2, hours, 3 hours and T_{Terminal}) and processed to plasma. Brains were collected at Tterminal (blotted, weighed, and snap frozen) and brain exposure was compared.

Genfitinib concentrations were quantitated by LC-MS/MS from brain and plasma from Group 1 animals. Compound 1 and metabolite (M38) concentrations were quantitated by LC-MS/MS from brain and plasma from Group 2. Non-compartmental analysis was performed using Phoenix WinNonlin or Watson LIMS with PK parameters provided (when calculable) including, but not limited to estimated concentration at T=0 (C0), total body clearance (CL), volume of distribution predicted at steady state (Vss), elimination half-life, maximal concentration (Cmax), time-to-reach maximal concentration (Tmax), area-underthe-curve with the dosing interval (AUC0-t); brain-to-plasma ratios were calculated.

Results of the study in orally- and IV-dosed mice are shown in Tables 3 and 4, respectively.

TABLE 3

| | | | | | | Brain | |
|---|---|---|---|---|---|---|---|
| | | | | Plasma | | Tissue | |
| | | | | Concentration | | Concen- | Brain/ |
| | | Dose | | by Hour | | tration | Plasma |
| Test | | (mg/ | Animal | (ng/mL) | | (ng/g) | (ratios) |
| Article | Route | kg) | ID | 3 hr | 7 hr | 7 hr | 7 hr |
| Gefitinib | PO | 50 | 1M001 | 1260 | 1030 | 562 | 0.546 |
| | | | 1M002 | 2660 | 2240 | 561 | 0.250 |
| | | | 1M003 | 1940 | 4700 | 666 | 0.142 |
| | | | Mean | 1953 | 2657 | 596 | 0.224 |
| | | | SD | 700 | 1870 | 60.3 | 0.0322 |
| | | | n | 3 | 3 | 3 | 3 |
| Com- | | 30 | 2M001 | 273 | 275 | 7400 | 26.9 |
| pound 1 | | | 2M002 | 181 | 265 | 8280 | 31.2 |
| | | | 2M003 | 229 | 197 | 5210 | 26.4 |
| | | | Mean | 228 | 246 | 6960 | 28.3 |
| | | | SD | 46 | 42 | 1580 | 3.73 |
| | | | n | 3 | 3 | 3 | 3 |

TABLE 4

| | | | | | | | | | Brain Tissue | Brain/ |
|---|---|---|---|---|---|---|---|---|---|---|
| | | Dose | | Plasma Concentration by Hour | | | | | Concentration | Plasma |
| Test | | (mg/ | Animal | (ng/mL) | | | | | (ng/g) | (ratios) |
| Article | Route | kg) | ID | 0.083 | 1 | 2 | 3 | 5 | 5 | 5 |
| Gefitinib | IV | 3 | 1M001 | 39.9 | 135 | 210 | 279 | 292 | 151 | 0.517 |
| (n = 6) | | | 1M002 | 38.2 | 125 | 166 | 206 | 282 | 149 | 0.528 |
| | | | 1M003 | 35.8 | 88.4 | 161 | 216 | 303 | 189 | 0.624 |
| | | | 1M004 | 55.6 | 115 | 161 | 225 | 286 | 155 | 0.542 |
| | | | 1M005 | 42.9 | 122 | 164 | 265 | 281 | 153 | 0.544 |
| | | | 1M006 | 39.8 | 116 | 180 | 252 | 237 | 129 | 0.544 |
| | | | Mean | 42.0 | 117 | 174 | 241 | 280 | 154 | 0.550 |
| | | | SD | 7.04 | 15.7 | 19.1 | 29.1 | 22.6 | 19.4 | 0.0377 |
| | | | n | 6 | 6 | 6 | 6 | 6 | 6 | 6 |
| Compound 1 | | | 2M007 | 14.3 | 63.0 | 70.1 | 120 | 119 | 2640 | 22.2 |
| (n = 6) | | | 2M008 | 14.2 | 63.5 | 93.4 | 95.5 | 107 | 2380 | 22.2 |
| | | | 2M009 | 13.5 | 54.8 | 84.2 | 93.7 | 120 | 2400 | 20.0 |
| | | | 2M010 | 12.8 | 59.6 | 81.6 | 106 | 109 | 2020 | 18.5 |
| | | | 2M011 | 13.1 | 44.2 | 70.7 | 89.5 | 97.4 | 2000 | 20.5 |
| | | | 2M012 | 11.3 | 51.1 | 71.9 | 94.7 | 109 | 1980 | 18.2 |
| | | | Mean | 13.2 | 56.0 | 78.7 | 99.9 | 110 | 2240 | 20.3 |
| | | | SD | 1.10 | 7.52 | 9.37 | 11.3 | 8.37 | 275 | 1.74 |
| | | | n | 6 | 6 | 6 | 6 | 6 | 6 | 6 |

### Example 7. Compound 1 Brain Penetration

Data obtained in a previously published report on brain penetration of EGFR TKIs in GMB was compared to brain penetrance data described herein (Table 5). The comparison indicates that gefitinib, erlotinib, afatinib, and visimpro failed to effectively treat GBM., and the effectiveness of osimertinib was not determined. Each of these drugs, with the exception of vizimpro, appeared to be a substrate of P-gp and Bcrp (efflux transporter proteins). In contrast, markedly increased brain penetrance was observed for Compound 1 relative the other drugs, which suggests that Compound 1 is not a substrate of the efflux transporter proteins.

TABLE 5
Brain Penetration Data

| | Brain Penetration | | |
|---|---|---|---|
| | (% of B/P ratio in | Efflux | |
| | preclinical model) | Liability | Glioblastoma |
| Gefitinib | 22%, 27%* | P-gp, Bcrp | Failed |
| Erlotinib | 13.7%* | P-gp, Bcrp | Failed |
| Afatinib | ND | P-gp, Bcrp | Failed |
| Vizimpro | ND | ND | Failed |
| Osimertinib | 180%* | P-gp, Bcrp | ND |
| Compound 1 | 2830% (oral) | Not a substrate | |
| | 2000% (infusion) | | |

ND = not determined;
*= Kim et al., Drug Metab. Dispos. 47(4):393-404 (2019).

### Example 8 Kinase Profiling of Compound 1

EGFR amplifications and/or mutations are common in GBM, estimated to occur in over 50% of patients. Accordingly, the binding affinity of Compound 1 for wt EGFR and several mutants was characterized and compared to that of the second-generation kinase inhibitor osimertinib (Table 6).

TABLE 6

| Gene Symbol | Osimertinib Kd (nM) | Compound 1 Kd (nM) |
|---|---|---|
| ALK | 69.00 | 250.00 |
| EGFR | 16.00 | 4.90 |
| EGFR(E746-A750del) | 1.70 | 0.70 |
| EGFR(G719C) | 22.00 | 2.20 |
| EGFR(G719S) | 21.00 | 1.70 |
| EGFR(L747-E749del, A750P) | 0.86 | 0.97 |
| EGFR(L747-S752del, P753S) | 8.10 | 3.10 |
| EGFR(L747-T751del,Sins) | 1.50 | 2.00 |
| EGFR(L858R) | 4.30 | 4.60 |
| EGFR(L858R,T790M) | 0.24 | 0.11 |
| EGFR(L861Q) | 3.50 | 3.90 |
| EGFR(S752-I759del) | 4.80 | 2.90 |
| EGFR(T790M) | 0.30 | 0.15 |
| ERBB2 | 6.00 | 11.00 |
| ERBB4 | 3.40 | 11.00 |
| PDGFRA | 740.00 | 1500.00 |
| PDGFRB | 1500.00 | 5200.00 |

### Example 9. Compound 1 has Lower Skin Toxicity than Osimertinib

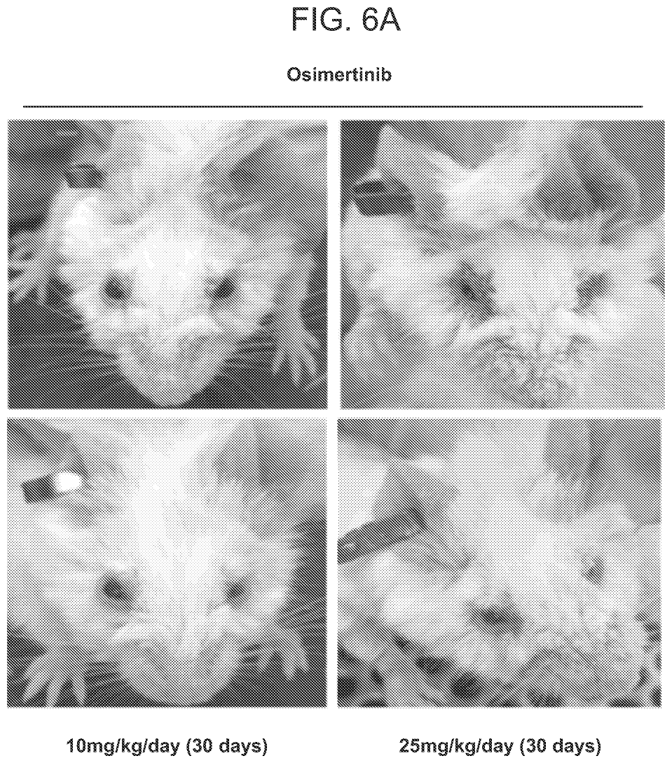

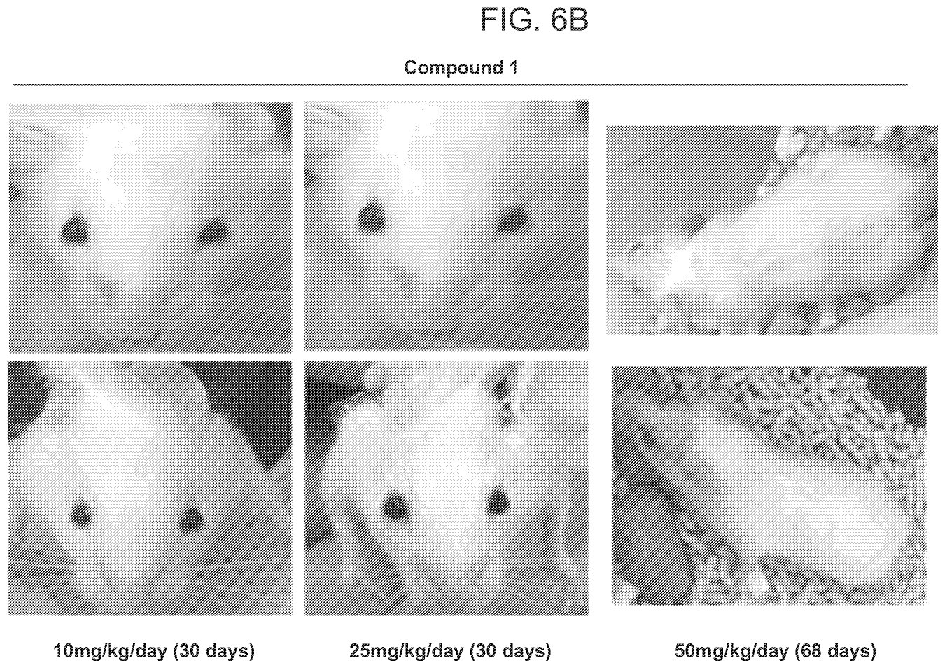

Mice were treated with osimertinib (10-25 mg/kg/day) or Compound 1 (10-50 mg/kg/day) and observed visually for skin toxicity. (osmertinib, FIG. 6A; Compound 1, FIG. 6B).

### Example 10. Compound 1 has a Larger Therapeutic Window than Osimertinib

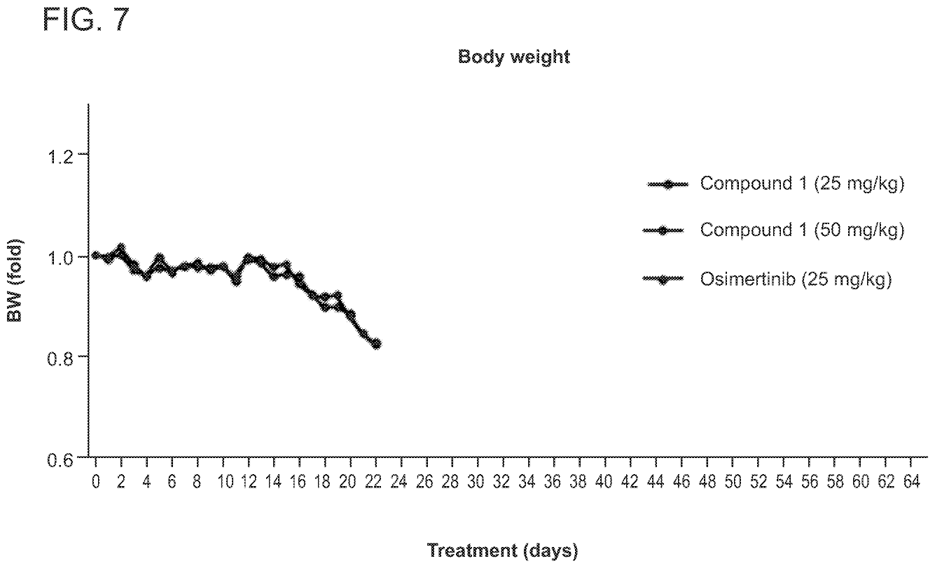

FIG. 7 shows body weight over the course of treatment for mice treated with Compound 1 (25-50 mg/kg) or osmertinib (25 mg/kg). Mice treated with osimertinib (25 mg/kg) reached the study endpoint within one month due to significant body weight loss.

### Example 11. Effects of Compound 1 and Osimertinib on NSCLC Brain Metastases

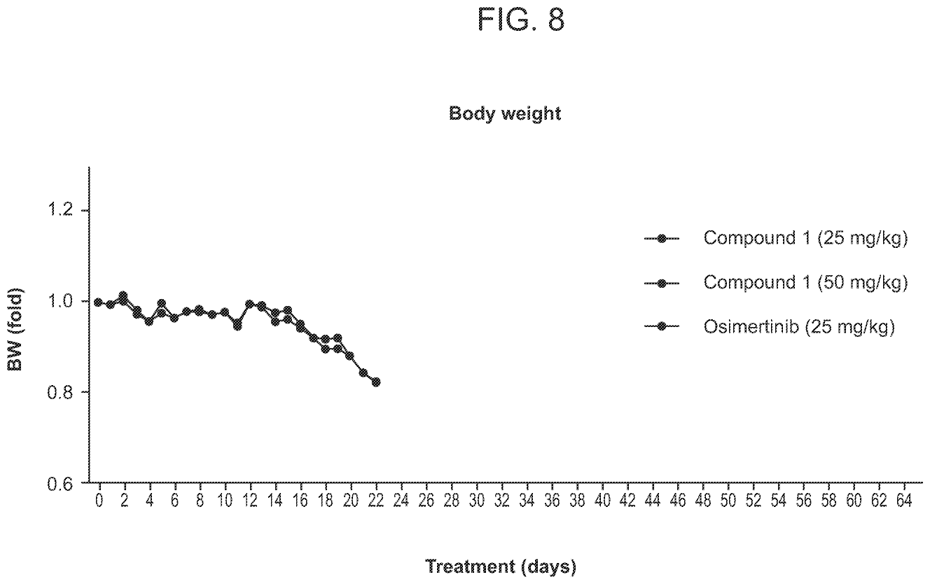

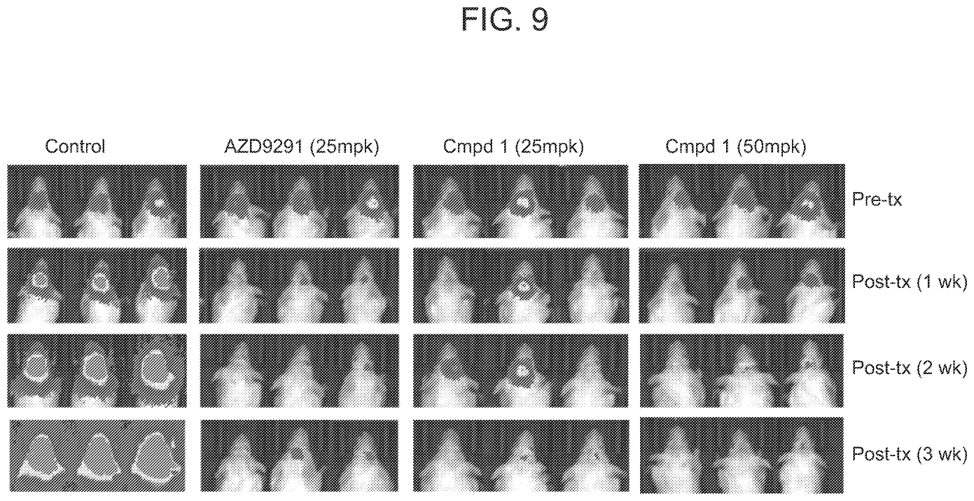

Female SCID mice bearing non small-cell lung cancer (NSCLC) brain metastases were dosed with Compound 1 (25-50 mg/kg) or osimertinib (AZD9291; 25 mg/kg). Reduced brain metastases were observed in all treated animals relative to control post-treatment. However, as shown in Table 1 and FIG. 8, mice in the AZD9291 group reached the study endpoint at 4 weeks due to body weight loss and skin lesions and therefore did not exhibit extended survival. However, mice treated with Compound 1 had median survival of 80.5 days (25 mg/kg) or over 100 days (50 mg/kg) (Table 7). These data indicate that Compound 1 has a larger therapeutic window for the treatment of NSCLC brain metastases (FIG. 9).

TABLE 7

| | | | Compound | Compound |
|---|---|---|---|---|
| | | AZD9291 | 1 | 1 |
| | Control | (25 mpk) | (25 mpk) | (50 mpk) |
| Median | 28.5 | 30 Days | 80.5 Days | >100 days |
| survival | Days | (due to body | | |
| | | weight loss) | | |

### Example 12: Imaging of Tissues Derived from Compound 1 Treated Mice

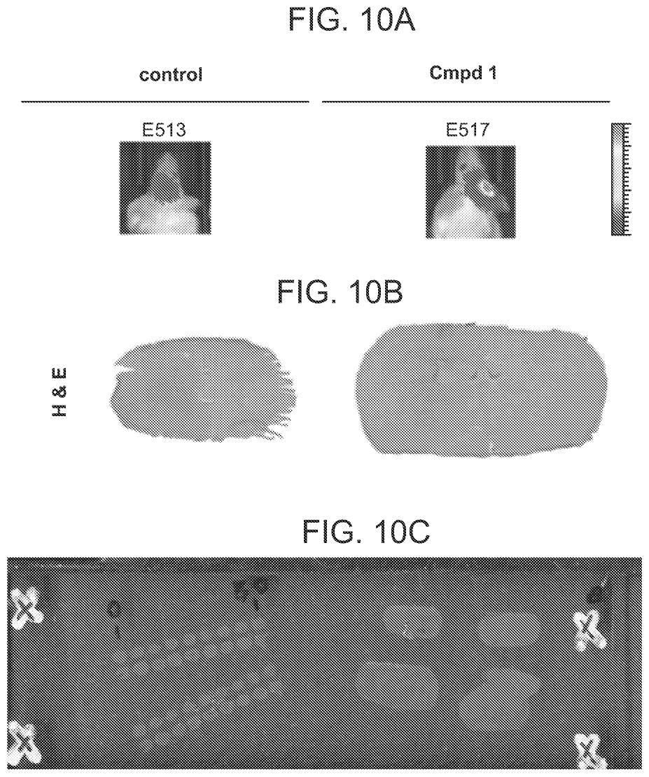

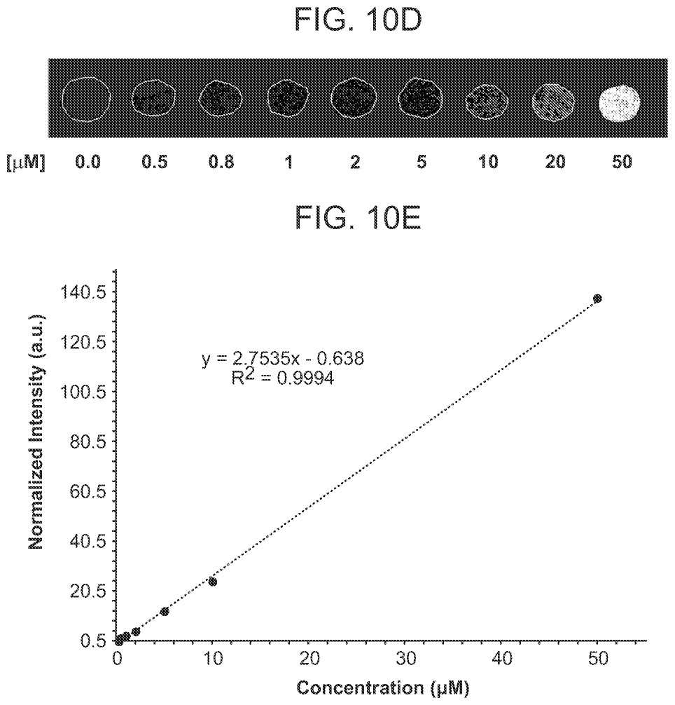

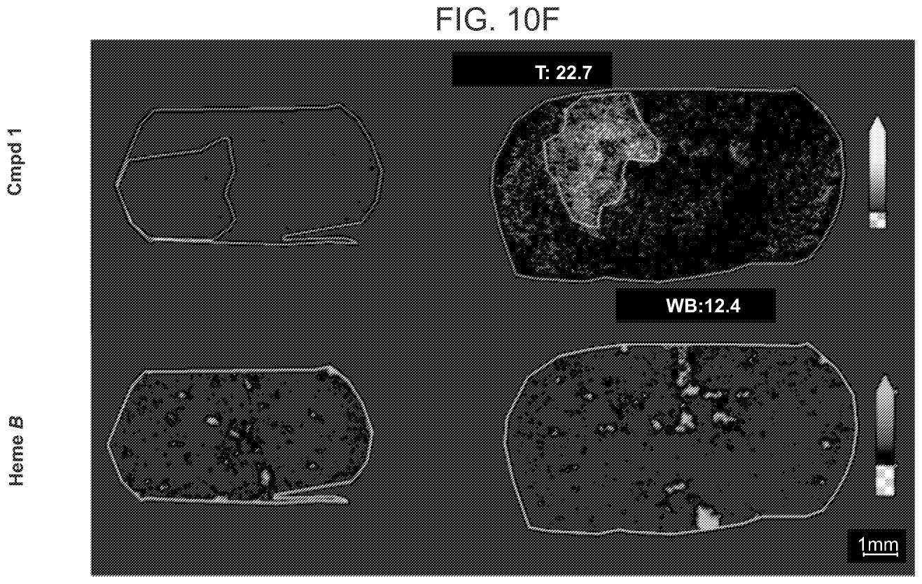

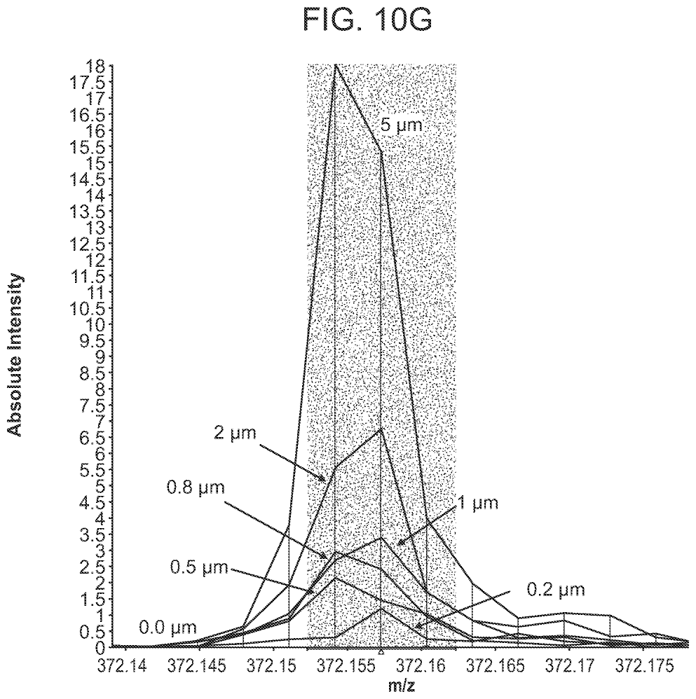

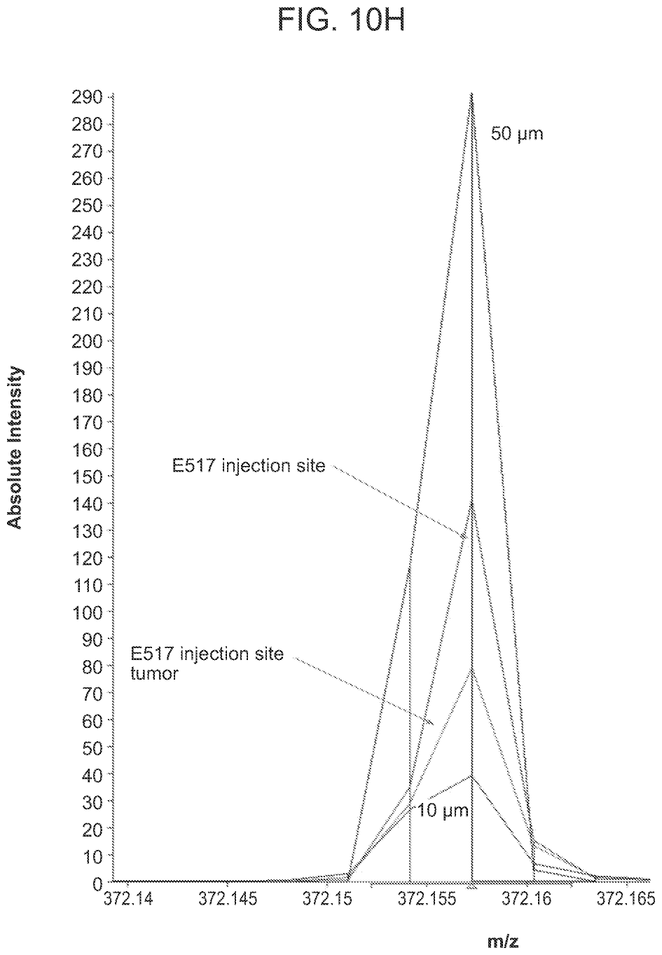

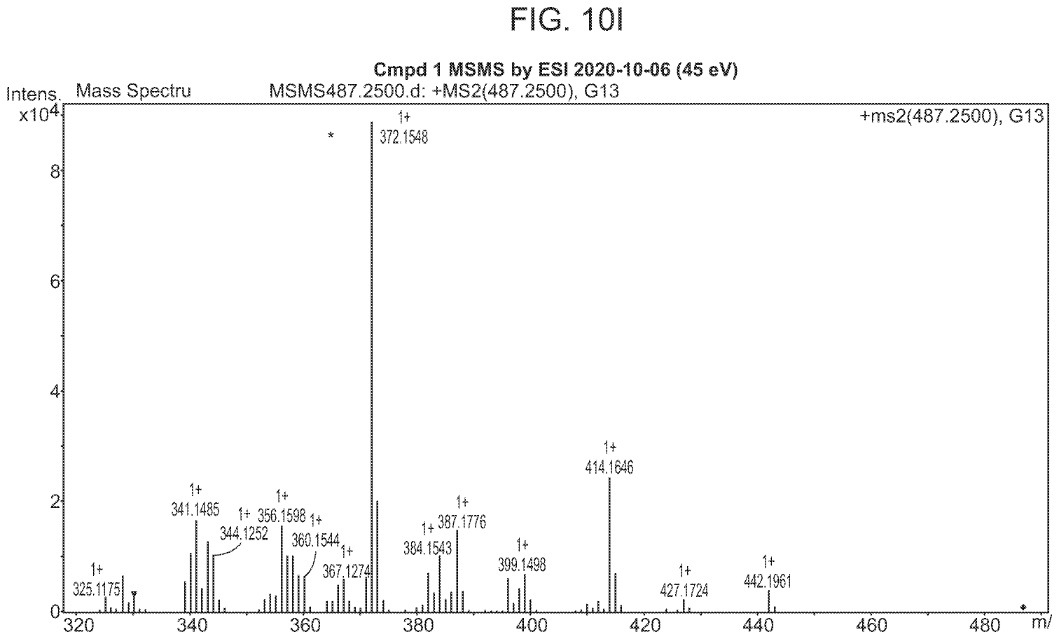

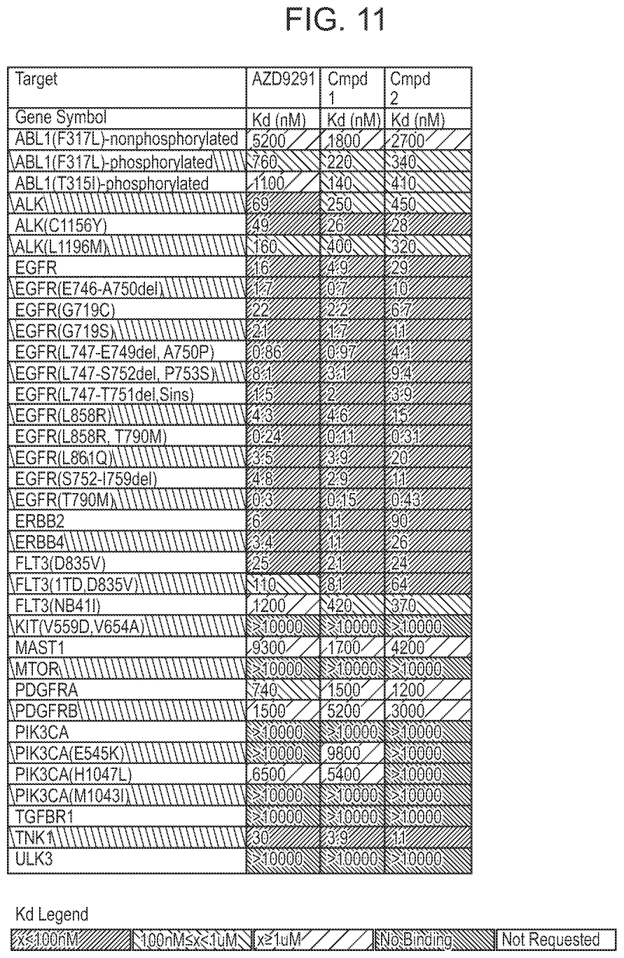

To image the drugs in brain tissues more sensitively, a matrix-assisted laser desorption ionization mass spectrometry imaging (MALDI-MSI) technique was employed. Briefly, GBM-bearing mice dosed with 100 mg/kg Compound 1 administered orally and control mice were sacrificed 7 hours post-treatment. Brain tissues were obtained and imaged using bioluminescence (FIG. 10A) and stained with hematoxylin and eosin (H&E) (FIG. 10B) to provide results to compare with images obtained using MALDI-MSI.

Tissue sections were prepared and deposited onto a MALDI matrix (80 mg/mL) super-DHB (sDHB) matrix, which is a 9:1 mixture of 2,5-dihydroxybenzoic acid (DHB) with 2-hydroxy-5-methoxybenzoic acid, 70:30 MeOH, and 0.1% trifluoroacetic acid (TFA). The Tissue-MALDI Sample Preparation System (TM-Sprayer) parameters were 75° C., a 0.18 mL/min flow rate at 10 psi.

A control mimetic was plated onto the MALDI substrate along with brain tissue sections from a control and a treated mouse (FIGS. 10C, 10D) and subjected to MALDI-MSI. FIG. 10E shows the normalized curve generated from the intensities observed for different concentration. FIG. 10F is an image of the intensities observed for the brain tissue samples. FIGS. 10G and 10H show the absolute intensities observed for the mimetics and for the brain tissue sample derived from the Compound 1 treated mouse. FIG. 10I shows an MSMS analysis of Compound 1.

### Example 13: Additional Testing

Compound 1 and its active/major metabolite, Compound 2, were evaluated in multiple in vitro and in vivo pharmacology studies to assess on target responses in lung and brain models. Kinase selectivity was also explored.

Compound 1 and/or Compound 2 (active/major metabolite) were evaluated for off-target activity in a large panel of receptors and ion channels, including a human ether-i-go-go-related gene (hERG) assay. Compound 1 was assessed comprehensively in Good Laboratory Practice (GLP) safety pharmacology studies, including studies of cardiovascular, respiratory and central nervous system function.

Pharmacokinetics/toxicokinetics (mouse/rat/dog), comparative protein binding, P450 inhibition and induction, transporter profiling, comparative in vitro metabolism and in vivo metabolism (rat) studies were conducted with Compound 1. CNS exposure was evaluated by oral and intravenous administration. The rat and dog are metabolically and pharmacologically relevant species for the nonclinical development program.

The toxicity of Compound 1 and via metabolism, its major metabolite Compound 2, were evaluated in dose-range and 4-week oral GLP studies in rats and dogs. All GLP studies were conducted using the tosylate salt, Lot Number A05993-056LB. The Phase 1 clinical study will be conducted with drug substance from Lot Number NB-Compound 1-A-3. The impurities in the clinical lot were qualified in the GLP toxicology studies. The drug product contains only neat drug substance in capsules.

### Pharmacology

Compound 1 has been thoroughly evaluated in primary, secondary and safety pharmacology studies as shown in Table 8.

TABLE 8
Summary of Pharmacology Studies

| | | | | |
|---|---|---|---|---|
| | | Dose | Dose (mg/kg) or | Report |
| Study Type | Species/System | Route | Concentration | Reference |

Primary Pharmacology

| | | | | |
|---|---|---|---|---|
| In vitro Activity with PC9GR4 and | PC9GR4 and | in vitro | n/a | Wang |
| H1975 Cells | H1975 cell lines | | | 2020 |
| In vivo Efficacy with H1975: | NOD.SCID Mice | PO | 3, 10, 30 | PRM-006 |
| Human Lung Tumor Cells | | | | |
| In vivo Efficacy with PC-9: Human | NOD.SCID Mice | PO | 3, 15, 50, 75 | PRM-007 |
| Lung Tumor Cells | | | | |
| Kinase Selectivity Binding | 468 kinases | in vitro | 1 μM free base | PRM-003 |
| Compound 1 and Compound 1-M38 | | | | |
| Targeted Kinase Binding Constants | Targeted kinases | in vitro | up to 30 μM free | PRM-004 |
| | | | base | |
| Summary of in vitro and in vivo | Multiple in vitro | in vivo/ | 0 to 5 μM in vitro | PRM-008; |
| pharmacology in glioblastoma | and in vivo assays | in vitro | 0, 37.5, 75 mg/kg | Ni 2021 |
| (GBM) | | | in vivo | |

Secondary Pharmacology

| | | | | |
|---|---|---|---|---|
| hERG Assay | HEK cells | in vitro | up to 30 μM free | PRM-001 |
| | | | base | |
| Receptor Selectivity Screen | Broad human | in vitro | 10 μM free base | PRM-005 |
| | receptor profile | | | |

Safety Pharmacology

| | | | | |
|---|---|---|---|---|
| Cardiovascular Function | Dog | PO | 0, 10, 30, 100 | PRM-002 |
| (Telemetry) | | | | |
| CNS-Functional Observational | Rat | PO | 0, 10, 30 100 | TOX-003 |
| Battery | | | | |
| Respiratory Rate | Dog | PO | 0, 10, 30, 100 | TOX-004 |

### Pharmacology Overview

Compound 1 and its active metabolite, Compound 2, were evaluated in multiple in vitro and in vivo studies to assess on target and off target pharmacology.

Compound 1 and Compound 2 (major metabolite) were evaluated in a panel of 468 human kinases and disease relevant mutant variants. Compound 1 and Compound 2 bound to few non-mutant kinases at <1% of control. The data suggest that Compound 1 and its major metabolite do not demonstrate significant non-selective kinase binding to wild-type proteins. Targeted non-mutant kinases included for Compound 1: MAST1, PAK4, PDGFRB and ULK3 and for Compound 1-M38: ERBB2, JAK3 (JH1-domain-catalytic), MKNK2, MTOR, OSR1 and TNK1. While there is some variability between Compound 1 and Compound 2, it is clear that both compounds are very effective at binding to a large panel of mutant kinases. Binding constants (Kd)<100 nM were confirmed for ALK, EGFR and FLT3 mutants. Binding constant to wild type EGFR was 4.9 nM. Binding constants <1 nM were apparent with EGFR (E746A-740del, L858R-T790M and T790M) double and single mutants for Compound 1; Compound 2 showed similar activity with EGFR (L858R-T790M; T790M) double and single mutants.

In HEK293 cells stably expressing EGFRvIII (most common EGFR variant in human GBM), Compound 1 reduced the phosphorylation of EGFRvIII (pEGFRvIII) at tyrosine sites 1068 and 1173 as well as phosphorylation of the downstream signaling molecules ERK1 and ERK2 (ERK1/2) in a dose-dependent manner. Further dose titration revealed an IC50 of 0.19 μM for Compound 1 on pEGFRvIII (Table 4-2). Compound 1 also reduced viability in HEK293-EGFRvIII cells with an IC50 of 1.48 μM. This was approximately the same as osimertinib, but 3-fold and 10-fold more active than erlotinib and gefitinib.

In a human GBM cell line (U251 cells) stably expressing EGFRvIII, Compound 1 decreased pEGFR with an IC50 of 0.17 μM (Table 9). In this study, decreased cell viability with Compound 1 (IC₅₀ 1.5 μM) approximately equaled that of osimertinib (IC₅₀ 2.6 μM); however, cell survival was 9-fold and 13-fold less than that of erlotinib and gefitinib.

In three primary patient-derived GBM cell lines harboring EGFR amplification and/or mutations in a neurosphere culture system, Compound 1 was the most potent inhibitor of cell viability with IC50 ranging from 0.89 to 3.28 μM, when compared to erlotinib, gefitinib or lapatinib.

TABLE 9
Compound 1 In Vitro Glioblastoma Activity

| Model | Target | Endpoint | Activity | Endpoint | Activity |
|---|---|---|---|---|---|
| HEK293 Cell | EGFRvIII | Reduce | 0.19 (μM) | Reduce cell | 1.48 (μM) |
| Line | | pEGFRvIII | | viability | |
| U251 Human | EGFRvIII | Reduce | 0.17 (μM) | Reduce cell | 1.5 (μM) |
| GBM Cell Line | | pEGFRvIII | | viability | |
| Three Patient | EGFR | n/a | n/a | Reduce cell | 0.89 to 3.28 (μM) |
| Cell Lines | amplification/ | | | viability | |
| | mutation | | | | |
| CPEVIII | EGFRvIII plus | Reduce | Marked | n/a | n/a |
| | Cdkn2a and | PEGFRvIII, | reduction in | | |
| | Pten double | pERK and | pEGFR, PERK. | | |
| | deletion | pS6RP | pS6RP | | |

To evaluate the in vivo activity of Compound 1 in GBM with mutant EGFR, U251 cells were generated to stably express EGFRvIII (U251-EGFRvIII) via retroviral-mediated gene transfer. U251-EGFRvIII cells were further engineered to express luciferase to facilitate monitoring drug response in vivo by bioluminescence-imaging analysis. In a pilot in vivo orthotopic study with U251-EGFRvIII cells (luciferase expressing) implanted in the brain, mice were treated from Day 28 post-implantation; luminescence signals were detected in the brain on Day 28. Compound 1 doses were 0 (n=7), 37.5 (n=8) and 75 (n=7) mg/kg/QD. There was no body weight loss during the study. Median survival was 68 days for controls, 80 days for low-dose and 89.5 days for high-dose Compound 1. These data suggest Compound 1 was safe and efficacious in this pilot study.

In GBM, EGFR mutations frequently coexist with a Cdkn2a deletion and Pten deficiency (Brennan 2013). In a genetically engineered mouse model of GBM driven by Cdkn2a and Pten double deletion concomitant with EGFRvIII expression (CPEvIII model), primary CPEvIII tumor cells were cultured as neurospheres and grafted intracranially in mice. Compound 1 markedly reduced phosphorylation of EGFRvIII, ERK1/2 and S6RP in a dose-dependent manner in vitro in neurosphere cultures. Compound 1 (75 mg/kg, QD) also prolonged the survival of mice bearing intracranial grafts of CPEvIII, with medium survival of 25.5 days for control mice and 33 days for Compound 1 treated mice (p=0.017) (Table 10). Immunohistochemistry analysis of pEGFR in tumors harvested from mice at termination revealed that phosphorylation levels of EGFRvIII were reduced in mice treated with Compound 1. Compound 1 was also well tolerated in this cohort of mice with no significant loss of body weight observed during Compound 1 treatment. Together, these results suggest that Compound 1 has activity against CPEvIII GBM tumor cells both in vitro and in vivo.

TABLE 10
Compound 1 In Vivo Glioblastoma Activity

| | | Oral Dose | |
|---|---|---|---|
| Model | Target | (mg/kg) | Survival |
| U251 cell | EGFRvIII | 0, 37.5, 75 | 0 mg/kg-68 days |
| xenografts | (luciferase | | 37.5 mg/kg-80 days |
| | expressing) | | (p < 0.05) 75 mg/kg-89.5 |
| | | | days (p < 0.01) |
| CPEvIII | EGFRvIII | 0, 75 | 0 mg/kg-25.5 days |
| genetically | plus Cdkn2a | | 75 mg/kg-33 days |
| engineered cell | and Pten double | | (p < 0.017) |
| xenografts | deletion | | |

One key feature of Compound 1 that sets it apart from all competitors is its extraordinary distribution to brain tissue. Compound 1's brain/plasma ratio is ˜20 in rats with continuous infusion. A recent study of brain distribution of competing EGFR-TKIs shows that gefitinib has 27% brain penetration, erlotinib 13.7%, and osimertinib 180%, (Kim 2019). The same study also showed that the 5 approved EGFR-TKIs are subject to extensive efflux transport. By contrast, current Crimson in vitro data show that Compound 1 is not likely a transporter substrate, rather a weak inhibitor of breast cancer resistant protein (BCRP) and no inhibition of P-glycoprotein (P-gp).

Together, the data suggest that Compound 1 has impressive in vitro and in vivo activity in models of GBM.

In in vitro non-small cell lung cancer (NSCLC) cell proliferation assays using PC9GR4 cells harboring the Ex19del/T790M double mutation and H1975 cells carrying the L858R/T790M double mutation, Compound 1 inhibited growth in the PC9GR4 cell line with an IC₅₀ of 3.66 nM and an IC₅₀ of 4.39 nM in H1975 cells (Wang 2020).

Compound 1 was also evaluated in xenograft models of lung tumor cell lines harboring EGFR single and double mutations in the mouse. Studies in NOD.SCID mice with SQ human lung implants (H1975 cells: EGFR L858R-T790M and PC-9 cells: EGFR Ex19Del) demonstrated that oral Compound 1 (30 or 50 mg/kg) was efficacious and safe. Anti-tumor activity was noted at Compound 1 plasma concentrations of 800 ng/mL (1.6 μM) and 1500 ng/mL (3.1 μM) at 4 hours post-dose in H1975 and PC-9 models, respectively. In H1975 lung tumor implants, substantial tumor regression (essentially cures) occurred over the course of the study.

Safety pharmacology studies demonstrate no respiratory or CNS risk. In the cardiovascular (CV) dog study, 1 of 4 dogs showed signs of a reversible ventricular conduction disturbance 10 to 24 hours following the high dose (100 mg/kg) of Compound 1. This arrythmia was not associated with maximum plasma concentration (Cₘₐₓ) or Tₘₐₓ of either parent or metabolite. No definitive link to the test article could be identified. Given the unexplained reversible arrythmia in this high-dose (100 mg/kg) telemetry dog, routine electrocardiogram (ECG) monitoring in Phase 1 is recommended.

Although the hERG IC₅₀ was 1.9 μM (925 ng/mL) with an IC₁₀₀˜30 μM, there were no QTc signals in a high-fidelity dog CV telemetry study (Spence 1998; Miyazaki 2002) with doses up to 100 mg/kg and an estimated single-dose Cₘₐₓ of 2618 ng/mL (5.4 μM) for Compound 1 and Cₘₐₓ˜367 ng/mL (0.75 μM) for Compound 1-M38; there were no adverse effects on blood pressure or cardiac intervals. There were no adverse CNS effects in the rat functional observational battery and there were no adverse effects on respiratory rate in the dog. Receptor binding studies suggest that Compound 1 has little off-target risk.

Compound 1 is efficacious in vitro and in vivo in mouse tumor models and relatively selective to the mutant kinases of interest. Safety pharmacology studies demonstrate no respiratory or CNS risk and no off-target receptor engagement. Given the unexplained arrythmia in one high-dose (100 mg/kg) telemetry dog and one high-dose (100 mg/kg) death (day 13) related to myocardial degeneration in the 4-week dog toxicology study, routine ECG monitoring through 8 hours post-dose to cover parent and metabolite and monitoring of cardiac enzymes (troponin) in Phase 1 is recommended along with pharmacokinetics.

The pharmacology and brain exposure profile are supportive of Phase 1 oncology dose-escalation studies in humans.

### Metabolism-Pharmacokinetics

The absorption, distribution, and metabolism of Compound 1 were explored in the studies outlined in Table 11.

TABLE 11
Summary of Metabolism-Pharmacokinetic Studies

| | | | | |
|---|---|---|---|---|
| | | Dose | Dose (mg/kg) or | Report |
| Study Type | Species/System | Route | Concentration | Reference |

Bioanalytical

| | | | | |
|---|---|---|---|---|
| LCMSMS-Compound 1 | Rat Plasma | NA | NA | MPK-014 |
| LCMSMS-Compound 2 | Rat Plasma | NA | NA | MPK-015 |
| LCMSMS-Compound 1 | Dog Plasma | NA | NA | MPK-016 |
| LCMSMS-Compound 2 | Dog Plasma | NA | NA | MPK-017 |

Pharmacokinetics and Absorption

| | | | | |
|---|---|---|---|---|
| Pharmacokinetics | Rat | IV, PO | 3, 10, 30 | MPK-001 |
| Oral Plasma/Brain Exposure | Rat | Oral | Compound 1: 30 | MPK-019 |
| | | | Gefitinib: 50 | |
| IV Plasma/Brain Exposure | Rat | IV | Compound 1: 3 | MPK-018 |
| | | | Gefitinib: 3 | |
| BCRP Inhibition | Caco-2 cells | in vitro | 0.1, 0.3, 1, 3, 10, 30 | MPK-011 |
| P-glycoprotein Inhibition | MDCK11-MDR1 | in vitro | 0.3, 1, 3, 10, 30, 100 | MPK-012 |
| | cells | | μM | |
| Permeation and Absorption | Caco-2 cells | in vitro | 0.5, 5, 50 μM | MPK-013 |

Distribution

| | | | | |
|---|---|---|---|---|
| Comparative Protein Binding | human, monkey dog, | in vitro | 0.5, 5, 50 μM | MPK-007 |
| | rat, mouse plasma | | | |

Metabolism

| | | | | |
|---|---|---|---|---|
| Comparative metabolic | human, monkey, dog, | in vitro | 1 μM | MPK-003 |
| stability | rat and mouse | | | |
| | hepatocytes | | | |
| Comparative metabolism | human, monkey, dog, | in vitro | 10 μM | MPK-004 |
| | rat and mouse | | | |
| | hepatocytes | | | |
| Metabolite Identification | rat dog | PO | 7.39 | MPK-005 |
| | monkey | | 3 | |
| | | | 3 | |
| CYP 1A2, 2B6 and 3A4 | human hepatocytes | in vitro | 0.03, 0.1, | MPK-008 |
| Induction | | | 0.3, 1 μM | |
| CYP450 Inhibition | human liver | in vitro | 0, 0.3, 1, 3, | MPK-009 |
| | microsomes | | 10, 30, 100 | |
| CYP Phenotyping | human liver | in vitro | 1 μM | MPK-010 |
| | microsomes | | | |

NA = not applicable;
LCMSMS = liquid chromatography-tandem mass spectrometry

Sensitive and reproducible liquid chromatography-tandem mass spectrometry (LC-MS/MS) assays were developed and validated to support metabolism-pharmacokinetic-toxicokinetic studies with Compound 1 and its major active metabolite, Compound 2.

Compound 1 is a moderately high clearance compound with a large volume of distribution in rats. In rats, oral Tₘₐₓ for Compound 1 was generally about ˜5 hours and for Compound 2 it was generally ˜7 hours; T_{1/2} was generally about 5 hours for Compound 1 and 9 hours for Compound 1-M38. There were no gender differences and bioavailability averaged 50% in rats and appeared independent of dose. Exposure was generally dose proportional.

Whole brain exposure of Compound 1 was 20-fold higher than plasma at estimated steady state by continuous intravenous infusion and oral dosing; Compound 2 brain exposure was essentially equal to plasma. There were no adverse effects noted in these studies.

Compound 1 was a weak inhibitor of rosuvastatin transport via human BCRP with an apparent IC₅₀ value of 3.02 μM (1471 ng/mL). Compound 1 did not inhibit P-gp mediated transport of digoxin (IC₅₀>30.0 μM). Compound 1 has moderate permeability in Caco-2 cells and is not likely a substrate of efflux transporters. Based on these data, there is generally a low risk of significant drug-drug interaction at therapeutic plasma concentrations via effects on these transporters. Further understanding of DDI risk will await definition of Phase 2 doses and exposures.

Compound 1 is primarily metabolized by CYP3A4/3A5; weak inhibition of CYP3A4-T, but not CYP3A4-M, was observed with an IC50 of 4.89 μM (2381 ng/mL). There was no CYP induction based on enzyme activity; based on mRNA there was CYP3A4 induction at 1 M, but not at lower concentrations. Metabolism, inhibition and induction were not observed with other CYPs. Potential DDI interactions exist with drugs that are inhibitors of CYP3A4-T or those metabolized by CYP3A4/3A5; these DDI risks will be better defined once there are human pharmacokinetics (PK) data.

Compound 1 was highly protein bound (95.6 to 98.7%) to plasma protein across species. Binding was independent of concentration.

In in vitro studies, metabolic stability was greatest in human hepatocytes; 22 metabolites were detected in human, monkey, dog, rat and/or mouse hepatocytes. Compound 2 was a major demethylated active metabolite in all species and with the exception of a minor metabolite, M48 in human hepatocytes, all human metabolites were represented in the rat and/or dog, the toxicology species.

In in vivo studies in rat, dog and monkey, 35 metabolites were identified. The major circulating compound in all species was parent compound; parent was also a major component in rat feces. Compound 2, demethylated metabolite, was a major active metabolite in all species in plasma and in rat feces. M34, glutathione conjugation and hydrolysis and acetylation metabolite, was a major metabolite in rat urine. Compound 1 was extensively metabolized during excretion with 31 metabolites in rat urine and 32 metabolites in rat feces.

Compound 1 has many metabolites and one major active metabolite in all species evaluated. Potential metabolic interactions exist with drugs that are inhibitors of CYP3A4-T or those metabolized by CYP3A4/3A5. The importance of these metabolic risks will be better defined once there are human PK data and targeted efficacious plasma concentrations are identified for Phase 2.

### Toxicology

Compound 1 has been tested in repeated-dose toxicity studies in rats and dogs for up to 4 weeks (Table 12). The design of the studies conducted under GLP was in full accordance with the relevant international guidelines. Dose formulation analytical methods were validated. The GLP toxicology program was completed with Compound 1 Lot Number A05993-056LB. The Phase 1 clinical study will be conducted with drug substance from Lot Number NB-Compound 1-A-3. All impurities in the clinical lot stored under accelerated conditions for 6 months were qualified in the toxicology program. The drug product is neat Compound 1 in capsules.

TABLE 12
Compound 1 Toxicology Program

| | | | | | |
|---|---|---|---|---|---|
| Study | | | | | |
| Type and | | Dose | | | Report |
| Duration | Species | Route | Dose (mg/kg) | GLP | Reference |

Repeat-dose Toxicity

| | | | | | |
|---|---|---|---|---|---|
| 14 days | Rat | PO | 30, 100, | N | TOX-002 |
| | | | 500, 1000 ª | | |
| | | | 0, 30, 100, 300 | | |
| 14 days | Dog | PO | 100, 300, | N | TOX-001 |
| | | | 600, 1000 ^{a} | | |
| | | | 0, 30, 100, 300 | | |
| 28 days | Rat | PO | 0, 10, 30, 100 | Y | TOX-003 |
| 28 days | Dog | PO | 0, 10, 30, 100 | Y | TOX-004 |
| Analytical | | | | | |
| Dose | NA | NA | NA | Y | TOX-006 |
| formulation | | | | | |
| validation | | | | | |

### Oral Rat Dose-Range Study

Male and female Sprague-Dawley rats were given Compound 1 orally by gavage either once (30, 100, 500, 1000 mg/kg/day) or once daily for up to 14 days (30, 100, 300 mg/kg/day). A single oral dose of Compound 1 was well tolerated at doses from 30 to 1000 mg/kg. When given for 14 days, morbidity and mortality, associated with marked decreases in food consumption and body weight (6.9%-F and 20.8%-M) were observed at 300 mg/kg; this group was terminated on Day 9. Lymphoid depletion was observed in thymus and spleen as was renal tubular vacuolation at this high dose. Similar but much less severe thymic lesions were noted at 100 mg/kg/day on Day 14. No significant toxicity was observed at 30 mg/kg/day. The STD₁₀ was estimated to be 100 mg/kg/day in this study.

### Oral Rat 4-Week Study

Compound 1 (0, 10, 30, and 100 mg/kg/day) was given orally, once daily by gavage to male and female Sprague-Dawley rats for up to 28 days. Morbidity, associated with marked body weight loss, necessitated termination of dosing in the high-dose group on Days 12/13. At this time, 5/sex in the high-dose group were taken to necropsy and 5/sex were started on recovery for the remainder of the study.

On Day 28, exposure to Compound 1 and Compound 2 increased with the increase in dose from 10 to 30 mg/kg; exposure as area under the curve (AUC) was generally dose proportional. There were no obvious sex differences or accumulation (Table 13).

TABLE 13
Summary of Rat Day 28 Mean Compound 1 and Compound 2
Toxicokinetic (TK)
Parameters

| | | | AUC₀₋₂₄ |
|---|---|---|---|
| Dose (mg/kg) | Sex | Cₘₐₓ (ng/mL) | (ng*hr/mL) |
| 10-Compound 1 | M | 109 | 1143 |
| 30-Compound 1 | M | 310 | 4353 |
| 10-Compound 2 | M | 48.9 | 708 |
| 30-Compound 2 | M | 100 | 1801 |
| 10-Compound 1 | F | 151 | 1798 |
| 30-Compound 1 | F | 300 | 4604 |
| 10-Compound 2 | F | 24.6 | 413 |
| 30-Compound 2 | F | 55.5 | 1079 |

Major histological findings at the interim necropsy (100 mg/kg) included generalized lymphoid depletion, pulmonary edema/inflammation and testicular seminiferous tubule degeneration. All findings except testicular degeneration were reversible (˜14 days) in this study; the reversibility period was not long enough to assess reversibility of testicular changes. On Day 29, only testicular degeneration was observed at and 100 mg/kg. No toxicity was noted at 10 mg/kg. The STD₁₀ was 30 mg/kg.

### Oral Dog Dose-Range Study

In the dog dose-range study, Compound 1 was given to male and female beagle dogs orally by gavage either once (30, 100, 600, 1000 mg/kg) or once daily for 14 days (30, 100, 300 mg/kg/day). A single oral dose of Compound 1 was well tolerated at doses from 30 to 1000 mg/kg. When given for 14 days, morbidity and mortality, associated with mild decreases in food consumption and marked decreases in body weight (7.6%-M and 10.6%-F) were observed at 300 mg/kg/day; thymic lymphoid depletion, hepatocellular pigment, renal tubular vacuolation and erosion in gastric fundus stomach were noted in these dogs. Similar but much less severe thymic and liver changes were noted at 100 mg/kg/day on Day 15.

No significant toxicity was observed at 100 mg/kg/day. The HNSTD was estimated to be 100 mg/kg/day in this study.

### Oral Dog 4-Week Study

Compound 1 (0, 10, 30, and 100 mg/kg/day) was given orally, once daily by gavage, to male and female beagle dogs (4/sex) for up to 28 days. In the high-dose group, one male was found dead on Day 13; abnormal clinical observations and decreased body weight at this dose necessitated suspension of dosing in the high-dose group on Day 18-M and Day 17-F. The high-dose survivors started a recovery period on Day 18/17 to Day 29. The cause of death in this high-dose male was attributed to myocardial degeneration. This lesion was not observed in any other dog on study. Increases in alkaline phosphatase, globulin, cholesterol, triglycerides and fibrinogen and decreased reticulocyte counts noted in the high-dose group at mid-study returned towards baseline by the end of the study following 11/12 days of recovery. Testicular changes at all doses (seminiferous epithelial vacuolation at 10 and 30 mg/kg and degeneration/atrophy at 100 mg/kg) were not reversible in this short-term study; the reversibility period was not long enough to assess reversibility of testicular changes.

Exposure to Compound 1 and Compound 2, assessed by Cₘₐₓ and AUC₀₋₂₄, generally increased as the dose increased on Day 1 and Day 28. The increases were generally dose-proportional or greater-than dose-proportional on Day 1 and Day 28. Gender difference was not observed. No significant accumulation of Compound 1 or Compound 2 was observed. These data are shown in Table 14.

TABLE 14
Summary of Dog Day 28 Mean Compound 1 and Compound 2 TK
Parameters

| | | | AUC₀₋₂₄ |
|---|---|---|---|
| Dose (mg/kg) | Sex | Cₘₐₓ (ng/mL) | (ng*hr/mL) |
| 10-Compound 1 | M | 578 | 3877 |
| 30-Compound 1 | M | 974 | 7180 |
| 10-Compound 2 | M | 143 | 1424 |
| 30-Compound 2 | M | 263 | 2975 |
| 10-Compound 1 | F | 867 | 6536 |
| 30-Compound 1 | F | 508 | 3490 |
| 10-Compound 2 | F | 242 | 3357 |
| 30-Compound 2 | F | 161 | 1791 |

No significant toxicities were observed at 10 or 30 mg/kg/day in females. The HNSTD was estimated to be 30 mg/kg/day.

### Integrated Nonclinical Efficacy and Safety Overview

GBM is the most common primary brain tumor in adults (Ostrom 2018). Many targeted therapies have demonstrated extensive success in other cancer types but have limited efficacy in GBM; the prognosis for patients with GBM remains grim (Kurz 2018; Miller and Wen 2016). More than 50% of GBMs have aberrant EGFR genetic variants. Most of these EGFR variants occur through mutations in the extracellular domain (Vivanco 2012). Among them, the most common EGFR variant (v), EGFRvIII (deletion of exon 2-7), has an in-frame extracellular domain truncation (Furnari 2015). It has been shown that EGFR-mutant GBM cells are likely addicted to EGFR signaling (An 2018; Huang 2009). Therefore, EGFR is an attractive therapeutic target in GBM.

Compound 1, a third-generation tyrosine kinase inhibitor, is under development for GBM (Wang 2020) Compound 1 binds covalently to its target. Compound 1 has demonstrated mutant kinase binding (Kd<1 nM) selectivity for several EGFR single and double mutants; there was little activity against wild-type EGFR. Compound 2 (major active metabolite) showed similar selective activity. In oral GBM mouse studies, Compound 1 appears safe and active at 37.5 and 75 mg/kg; IC₅₀ in in vitro studies targeting decreases in phosphorylated EGFR and cell viability ranged from 0.17 to 3.28 μM.

There were no adverse effects on blood pressure or cardiac intervals, including QT and QTc. One high-dose telemetry dog had a reversible junctional arrythmia from 12 to 24 hours post-dose that was not associated with Tₘₐₓ of either parent or metabolite, suggesting that it was not caused by Compound 1 or Compound 2. There were no adverse effects on CNS parameters or respiratory rate. Receptor binding studies suggest that Compound 1 has little off-target risk. Given the unexplained arrythmia in one high-dose telemetry dog routine ECG monitoring through 8 hours post-dose in Phase 1 is recommended.

Compound 1 is a moderately high-clearance compound with a large volume of distribution in rats. In rats, oral Tₘₐₓ for Compound 1 was generally about 5 hours and for Compound 2 it was generally 7 hours; T_{1/2} was generally about 5 hours for Compound 1 and 9 hours for Compound 2. There were no gender differences and bioavailability averaged 50% in rats and appeared independent of dose. Exposure was generally dose proportional. Compound 1 was highly protein bound to plasma protein across species; binding was independent of concentration.

Compound 1 has many metabolites and one major active metabolite (Compound 2) that was identified in all species evaluated. Potential metabolic interactions exist with drugs that are inhibitors of CYP3A4-T or those metabolized by CYP3A4/3A5. The importance of these metabolic risks will be better defined once there are human PK data and targeted efficacious plasma concentrations are identified in humans.

In a 4-week toxicity study in rats, there was mortality at 100 mg/kg; major histological findings at an interim necropsy (100 mg/kg) included generalized lymphoid depletion, pulmonary edema/inflammation and testicular seminiferous tubule degeneration. All findings except testicular degeneration were reversible in this study; the reversibility period was not long enough to assess reversibility of testicular changes. On Day 29, testicular degeneration was observed at 30 and 100 mg/kg. No toxicity was noted at 10 mg/kg. The STD₁₀ was 30 mg/kg.

In a 4-week toxicity study in dogs, there was one death at 100 mg/kg attributed to myocardial degeneration on Day 12; this was the only high-dose dog with myocardial lesions. Testicular changes noted at all doses (seminiferous epithelial vacuolation at 10 and 30 mg/kg and degeneration/atrophy at 100 mg/kg), were not reversible; the reversibility period was not long enough to assess reversibility of testicular changes. No significant toxicities were observed at 10 or 30 mg/kg/day in females. The HNSTD was estimated to be 30 mg/kg/day.

Testicular lesions were noted in dogs and rats; a NOEL was not established in dogs and it was 10 mg/kg in rats; the studies were not of sufficient duration to study reversibility. Reversible junctional arrhythmias in one dog and myocardial lesions leading to death in another dog were noted at 100 mg/kg; the NOEL for myocardial risk was 30 mg/kg in the dog and 100 mg/kg in the rat. These potential risks should be identified in the informed consent document and considered in starting dose calculations. ECG monitoring through 8 hours post-dose to cover parent and metabolite and cardiac troponin assessments are recommended in the Phase 1 clinical program.

### Phase I Starting Dose Recommendation

Based on pharmacological potency and on the nonclinical safety pharmacology, metabolism and pharmacokinetic studies and dose responses, Compound 1 is expected to be a relatively safe and efficacious compound at therapeutic doses in oncology patients. The toxicology program in the dog (HNSTD=30 mg/kg) supports a starting dose up to 300 mg/60 kg based on oncology dose-selection guidance. Toxicology data in the rat (STD₁₀=30 mg/kg) support a starting dose up to 180 mg/60 kg based on oncology dose-selection guidance.

Testicular lesions were observed in both the rat and dog from 30 mg/kg and 10 mg/kg, respectively. Myocardial risks were described at 100 mg/kg in the dog but not at 30 mg/kg; the NOEL for myocardial risk is 30 mg/kg (human equivalent dose [HED]=15 mg/kg). Based on these data, the starting dose would be 1.5 mg/kg or 90 mg/60 kg for this oral compound.

The recommended starting dose for Phase 1 oncology patients is 100 mg QD.

### Nonclinical Highlights

Pharmacologically active in GBM mouse models at 37.5 and 75 mg/kg QD orally
Pharmacologically active in NSCLC mouse models at 30 and 50 mg/kg OD orally
   Activity was observed at plasma concentrations of 800 ng/mL (1.6 μM) and 1500 ng/mL (3.1 μM) at 4 hours post-dose
Many metabolites (22 in human, monkey, dog, rat and/or mouse hepatocytes) and one major active metabolite (Compound 2) were identified in all species evaluated including in human hepatocytes
Moderate high-clearance with a large volume of distribution in rat
Oral Tₘₐₓ was generally about 5 hours and 7 hours for Compound 1 and Compound 2
T_{1/2} was generally about 5 and 9 hours for Compound 1 and Compound 2
Highly bound to plasma protein across species and independent of concentration
Absolute bioavailability was ˜50% in rats
Whole brain exposure of Compound 1 was 20-fold higher than plasma at estimated steady state by continuous intravenous infusion and oral dosing in the rat; Compound 2 brain exposure was essentially equal to plasma
Potential metabolic interactions exist with drugs that are inhibitors of CYP3A4-T or those metabolized by CYP3A4/3A5
Weak inhibitor of rosuvastatin transport via human BCRP with an apparent IC₅₀ value of 3.02 μM (1471 ng/mL)
No P-gp inhibition
Moderate permeability in Caco-2 cells and not a likely substrate of efflux transporters
No adverse effects on heart rate, blood pressure or cardiac intervals, including QT and QTc in the dog
   one high-dose telemetry dog had a reversible junctional arrythmia from 12 to 24 hours post-dose that was not associated with Tₘₐₓ of either parent or metabolite, suggesting that it was not caused by Compound 1 or Compound 2
Testicular degeneration in rats and dogs; studies were not of sufficient duration to assess reversibility
Myocardial lesions leading to death in one high-dose dog on day 12 (100 mg/kg)
NOEL for myocardial risk was 30 mg/kg in the dog and 100 mg/kg in the rat
4-week Dog toxicology no observed adverse effect level (NOAEL)-10 mg/kg
4-week Rat toxicology NOAEL-10 mg/kg
STD₁₀ was 30 mg/kg in rats
HNSTD was 30 mg/kg in dogs

### Dosage and Administration

Compound 1 is administered orally as a single daily dose (although alternative frequencies or intermittent schedules may be instigated in response to emerging safety, tolerability, or PK data). Dosing should occur at approximately the same time each day on an empty stomach (i.e., at least 1 hour before or 2 hours after eating). The initial daily dose in the first-in-human trial will be 100 mg.

The toxicology program in the dog (HNSTD=30 mg/kg) supports a starting dose up to 300 mg/60 kg based on oncology dose-selection guidance. Toxicology data in the rat (STD₁₀=30 mg/kg) support a starting dose up to 180 mg/60 kg based on oncology dose-selection guidance.

Testicular lesions were observed in both the rat and dog from 30 mg/kg and 10 mg/kg, respectively. Myocardial risks were described at 100 mg/kg in the dog but not at 30 mg/kg; the NOEL for myocardial risk is 30 mg/kg (HED=15 mg/kg). Based on these data, the starting dose would be 1.5 mg/kg or 90 mg/60 kg.

The recommended starting dose for Phase 1 oncology patients is 100 mg QD.

### Example 14

Drug delivery across the blood-brain barrier (BBB) is a major obstacle that all EGFR-targeting agents for brain cancers have to face. Numerous EGFR-TKIs have been evaluated for the treatment of GBM unsuccessfully, despite evidence that EGFR signaling is required for the viability of EGFR-mutant GBM cells (Westphal, M., et al. CNS Drugs 31, 2017, 723-735). Many of these inhibitors fail to cross the BBB or are substrates of drug efflux pumps, and often have a relatively small therapeutic window.

To determine the brain penetration of Compound 1, a comparative assessment of brain exposure of Compound 1 and gefitinib was performed seven hours following a single oral dose of in rats with Compound 11 (30 mg/kg) or gefitinib (50 mg/kg). The results showed that Compound 11 brain/plasma (B/P) ratio was 28.3 and the B/P ratio for gefitinib was 0.22 (Tables 3 and 4). A more in-depth comparative assessment was carried out on brain exposure of Compound 11 and gefitinib following continuous intravenous infusion to estimated steady state in rats. Both compounds were infused to a total dose of 3 mg/kg over five hours. Results showed that Compound 11 B/P ratio was 20.3 and gefitinib was 0.55, consistent with the data from oral administration of the drugs. Notably, plasma concentrations of Compound 11 are much lower than that of gefitinib at all time points in both oral and intravenous administrations (Tables 3 and 4). These data confirm that Compound 11 is an ideal compound for treating GBM as it is preferentially present at high concentrations in brain with no apparent adverse effects in these pilot studies.

Compound 11 was a weak inhibitor of rosuvastatin transport via human breast cancer resistance protein (BCRP), with an apparent IC50 value of 3.02 μM. Compound 11 did not inhibit P-glycoprotein (P-gp)-mediated transport of digoxin (IC50>30.0 μM). Compound 11 also showed moderate permeability in Caco-2 cells. Based on these data, it is unlikely that Compound 11 is a substrate of efflux transporters, and there is generally a low risk of significant drug-drug interaction at therapeutic plasma concentrations via effects on these transporters.

Remarkably, Compound 11 distributes and accumulates in the brain at levels that are approximately 20-fold in excess of blood plasma levels. Unlike other EGFR-TKIs, which are subject to efflux transporters, Compound 11 is not a substrate of P-gp- or BCRP-mediated drug transport functions; instead, it is a modest inhibitor of BCRP. Moreover, Compound 11 exhibits a relatively high clearance rate to maintain relatively low plasma levels.

A recent study showed that, while EGFR is important for brain development during embryonic and early postnatal stages, adult mice with brain-specific deletion of EGFR appear to be normal (Robson, J. P., et al. The FEBS Journal 285, 2018, 3175-3196), suggesting that EGFR inhibition in the CNS will not lead to dose-limiting toxicity. Thus, the distinct pharmacologic properties of Compound 11, in particular its high brain/plasma ratio, will potentially provide a “tissue-based” therapeutic window to allow effective inhibition of EGFR in the tumor, while relatively sparing the receptor systemically. Pre-clinical data demonstrated that Compound 11 provides a sufficiently wide therapeutic window to effectively inhibit EGFRvIII intracranially without significant extracranial toxicity.

### Example 15: Preparation of Salt of N-(5-((4-(1H-pyrrolo[2,3-b]pyridin-1-yl)pyrimidin-2-yl)amino)-4-methoxy-2-(methyl(2-(methylamino)ethyl)amino)phenyl)acrylamide (Salt of Compound 12)

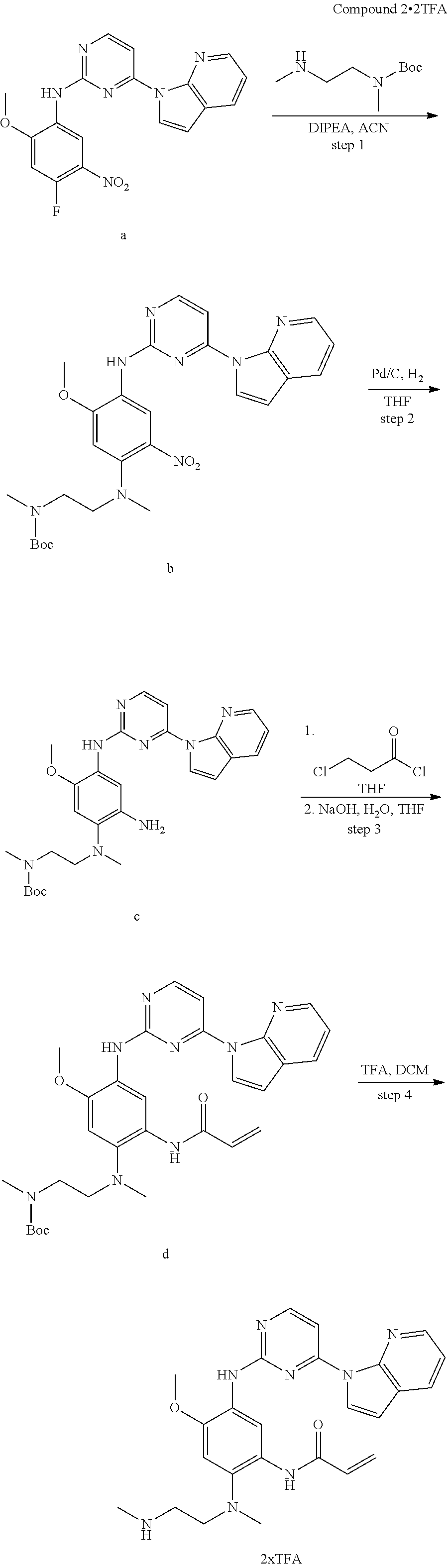

### Step 1. Synthesis of tert-butyl (2-((4-((4-(1H-pyrrolo[2,3-b]pyridin-1-yl)pyrimidin-2-yl)amino)-5-methoxy-2-nitrophenyl)(methyl)amino)ethyl)(methyl)carbamate (Compound b)

To a solution of N-(4-fluoro-2-methoxy-5-nitrophenyl)-4-(1H-pyrrolo[2,3-b]pyridin-1-yl)pyrimidin-2-amine (Compound a; 17.2 g, 1.0 eq.) in ACN (260 mL) were added tert-butyl methyl(2-(methylamino)ethyl)carbamate (12.7 g 1.5 eq.) and DIPEA (11.7 g, 2.0 eq.) at 20° C. and the resulting mixture was stirred at 80° C. for 24 h. The reaction mixture was cooled to 38° C., add 1120 (200 mL) and stirred for additional 30 min. The resultant mixture was filtered through celite and the filter cake was rinsed with H₂O and then with ACN. The filter cake was dried at 40˜45° C. (tank temperature) under vacuum to give crude Compound b (23.0 g, 93% yield, 97.9% purity determined by HPLC) as red solid. Rₜ=5.329 min. MS m/z: 225.2 [(M−Boc)/2+1].

### Steps 2 and 3. Synthesis of tert-butyl (2-((4-((4-(1H-pyrrolo[2,3-b]pyridin-1-yl)pyrimidin-2-yl)amino)-2-acrylamido-5-methoxyphenyl)(methyl)amino)ethyl)(methyl)carbamate (Compound d)

To a solution of Compound b (16 g, 1.0 eq.) in THF (160 mL) was added Pd/C (0.8 g, 5 wt %) and the resultant mixture was vacuumed to ≤−80 KPa and then inflated with hydrogen to atm for three times. The hydrogenation reaction was kept at 25±5° C. for 42 h. The reaction mixture was filtered through celite and the filter cake was rinsed with THF. The filtrate was cooled to 0-5° C. and added 3-chloropropanoyl chloride while keeping the reaction mixture at 0-5° C. After 15 min, yellow solid precipitated out. LC-MA analysis showed that an intermediate, tert-butyl (2-((4-((4-(1H-pyrrolo[2,3-b]pyridin-1-yl)pyrimidin-2-yl)amino)-2-amino-5-methoxyphenyl)(methyl)amino)ethyl)(methyl)carbamate (Compound c), was completed gone in 1 h. The reaction mixture was added a solution of NaOH (4.7 g, 4.0 eq.) in water (128 mL) at 20-25° C. and stirred for 21 h. The organic phase was separated and concentrated under reduced pressure at a temperature below 40° C. The concentrated organic phase was added ethyl acetate and washed with water. The organic layer was collected and concentrated under reduced pressure at a temperature below 40° C. Purification by chromatography (silica gel 200-300 mesh, PE:EA=3:1) provided Compound d (10 g, 60% yield, 94.6% purity determined by HPLC) as light yellow solid. Rₜ=4.221 min. MS m/z: 573.2 [M+1].

### Step 4. Synthesis of N-(5-((4-(1H-pyrrolo[2,3-b]pyridin-1-yl)pyrimidin-2-yl)amino)-4-methoxy-2-(methyl(2-(methylamino)ethyl)amino)phenyl)acrylamide bis(2,2,2-trifluoroacetate) (Compound 2-2TFA)

To a solution of Compound d (3.0 g, 1.0 eq) in DCM (45 mL) at 20-25° C. was added TFA (11.1 g, 18.6 eq.). After 18 h, the reaction mixture was concentrated under vacuum at 40° C. until dry. The concentrated residue was dissolved in EA and then added saturated sodium bicarbonate solution until PH is 7-8. The resultant mixture was filtered through celite and the filter cake was rinsed with H₂O then ACN. The filter cake was dried at 40-45° C. (tank temperature) under vacuum to afford Compound 2-2TFA (2.0 g, 80.6% yield, 97.8% purity determined by HPLC) as red solid. Rₜ=13.299 min. MS m/z: 473.2 [M+1]. ¹H NMR (300 MHz, DMSO-d6) δ 9.31 (s, 1H), 8.92 (s, 1H), 8.73 (d, J=4.0 Hz, 1H), 8.63-8.53 (m, 3H), 8.52-8.40 (m, 2H), 8.20 (s, 1H), 8.13 (dd, J=7.8, 1.6 Hz, 1H), 7.33 (dd, J=7.8, 4.8 Hz, 1H), 7.00 (s, 1H), 6.83-6.67 (m, 2H), 6.36 (dd, J=16.9, 2.1 Hz, 1H), 5.81 (dd, J=10.1, 2.1 Hz, 1H), 3.90 (s, 3H), 3.22 (t, J=5.7 Hz, 2H), 3.14 (d, J=5.7 Hz, 2H), 2.64 (s, 3H), 2.60 (s, 3H).

### Example 16: Cell Viability Assays for AZD9291 and Compound 1

Cell viability assays for known EGFR inhibitor AZD9291 and Compound 1 described herein were performed following the procedures set forth in the General Biological Assay A, Cell Viability Assay. CellTier-Glo (Promega) assay kit (https://www.promega.com/resources/protocols/technical-bulletins/0/celltiter-glo-luminescentcell-viability-assay-protocol/) was used here. NCI-H1975 cells (EGFR L858R/T790M mutation) and PC-9 cells (EGFR exon 19 deletion) were used in the cell viability assays.

AZD9291 was obtained from commercial source. Compound 2 was synthesized as described in Example 15. Compound 1 was synthesized according to the methods disclosed in Gray et al., U.S. Patent Application Publication No.: 2017/0362204 A1.

NCI-H1975 cells (EGFR L858R/T790M mutation) were incubated with each of AZD9291 and Compound 1 for 72 hours. AZD9291 exhibited IC₅₀ of 12.93 nM. Compound 1 exhibited IC₅₀ of 34.39 nM.

PC-9 cells (EGFR exon 19 deletion) were incubated with each of AZD9291, Compound 1. AZD9291 exhibited IC₅₀ of 18.12 nM. Compound 1 exhibited IC₅₀ of 62.78 nM.

In the cell viability assays, Compound 1 showed excellent potency in inhibiting both EGFR with L858R/T790M mutation and EGFR with exon 19 deletion.

### Example 17: In vitro EGFR/ERK Phosphorylation Assays for AZD9291 and Compound 1

The EGFR/ERK Phosphorylation Assays were performed following known procedures. NCI-H1975 cells (EGFR L858R/T790M mutation) and PC-9 cells (EGFR exon 19 deletion) were used in the phosphorylation assays.

NCI-H1975 cells (EGFR L858R/T790M mutation) were treated with each of AZD9291 and Compound 1 for 6 hours. EGFR signaling pathway was determined by Western blot analysis.

PC-9 cells (EGFR exon 19 deletion) were treated with each of AZD9291 and Compound 1 for 6 hours. EGFR signaling pathway was determined by Western blot analysis.

### Example 18: Toxicokinetic Analyses of EGFR Inhibitor Compound 1

Thirty-two beagle dogs (conventional, naive) were divided into four groups. Each group consists of four male and four female. Group 1 beagle dogs were administered vehicle and groups 2-4 beagle dogs were administered Compound 1 at 10 mg/kg, 30 mg/kg, and 100 mg/kg, respectively, by oral gavage once daily for 28 consecutive days (Table 15). T_{1/2} of 8 h and Tₘₐₓ of 4 h were determined for Compound 1 on day 1. Cₘₐₓ (ng/mL) and AUCₗₐₛₜ (hr*ng/mL) of Compound 1 on day 1 were obtained for groups 2-4 (Table 15).

TABLE 15

| | | | | TK Parameters | |
|---|---|---|---|---|---|
| Study | Group | Dose Level | | Cₘₐₓ | AUCₗₐₛₜ |
| Day | No. | (mg/kg/day) | Sex | (ng/ml) | (hr*ng/ml) |
| 1 | 2 | 10 | Male | 166 | 1648 |
| | | | Female | 142 | 2060 |
| | 3 | 30 | Male | 317 | 3789 |
| | | | Female | 170 | 1959 |
| | 4 | 100 | Male | 367 | 5187 |
| | | | Female | 293 | 4055 |

Compound 1 and its active/major metabolite, Compound 2, were evaluated in multiple in vitro and in vivo pharmacology studies to assess on target responses in lung and brain models. Kinase selectivity was also explored.

Compound 1 and/or Compound 2 (active/major metabolite) were evaluated for off-target activity in a large panel of receptors and ion channels, including a human ether-i-go-go-related gene (hERG) assay. Compound 1 was assessed comprehensively in GLP safety pharmacology studies, including studies of cardiovascular, respiratory and central nervous system function.

Pharmacokinetics/toxicokinetics (mouse/rat/dog), comparative protein binding, P450 inhibition and induction, transporter profiling, comparative in vitro metabolism and in vivo metabolism (rat) studies were conducted with Compound 1. CNS exposure was evaluated by oral and intravenous administration. The rat and dog are metabolically and pharmacologically relevant species for the nonclinical development program.

The toxicity of Compound 1 and via metabolism, its major metabolite Compound 2, were evaluated in dose-range and 4-week oral GLP studies in rats and dogs. All GLP studies were conducted using the tosylate salt.

### Pharmacology and Activity Profiles

Comparative activity profiling studies, shown in FIG. 11, were conducted which showed that Compound 1 being superior or competitive to osimertinib. (See also, Ni, J. et al. 2021 “Targeting EGFR in glioblastoma with a novel brain-penetrant small molecule EGFR-TKI” bioRxiv preprint doi: https://doi.org/10.1101/2021.01.09.426030.)

Compound 1 and its active metabolite, Compound 2, were evaluated in multiple in vitro and in vivo studies to assess on target and off target pharmacology.

Compound 1 and Compound 2 (major metabolite) were evaluated in a panel of 468 human kinases and disease relevant mutant variants. Compound 1 and Compound 2 bound to few non-mutant kinases at <1% of control. The data suggest that Compound 1 and its major metabolite do not demonstrate significant non-selective kinase binding to wild-type proteins. Targeted non-mutant kinases included for Compound 1: MAST1, PAK4, PDGFRB and ULK3 and for Compound 2: ERBB2, JAK3 (JH1-domain-catalytic), MKNK2, MTOR, OSR1 and TNK1. While there is some variability between Compound 1 and Compound 2, it was clear that both compounds are very effective at binding to large panel of mutant kinases. Binding constants (Kd)<100 nM were confirmed for ALK, EGFR and FLT3 mutants. Binding constant to wild type EGFR was 4.9 nM. Binding constants <1 nM were apparent with EGFR (E746A-740del, L858R-T790M and T790M) double and single mutants for Compound 1; Compound 2 showed similar activity with EGFR (L858R-T790M; T790M) double and single mutants.

Safety pharmacology studies demonstrate no cardio-respiratory or CNS risk.

There were no adverse CNS effects in the rat functional observational battery and there were no adverse effects on respiratory rate in the dog. Receptor binding studies suggest that Compound 1 has little off-target risk.

### Metabolism-Pharmacokinetics

Sensitive and reproducible LC-MS/MS assays were developed and validated to support metabolism-pharmacokinetic-toxicokinetic studies with Compound 1 and its major active metabolite, Compound 2.

Compound 1 is a moderately high clearance compound with a large volume of distribution in rats. In rats, oral Tₘₐₓ for Compound 1 was generally about ˜5 hours and for Compound 2 it was generally ˜7 hours; T_{1/2} was generally about 5 hours for Compound 1 and 9 hours for Compound 2. There were no gender differences and bioavailability averaged 50% in rats and appeared independent of dose. Exposure was generally dose proportional.

Whole brain exposure of Compound 1 was found to be about 20-fold higher than plasma at estimated steady state by continuous intravenous infusion and oral dosing; Compound 2 brain exposure was essentially equal to plasma. There were no adverse effects noted in these studies.

Compound 1 is primarily metabolized by CYP3A4/3A5; weak inhibition of CYP3A4-T, but not CYP3A4-M, was observed with an IC50 of 4.89 μM (2381 ng/mL). There was no CYP induction based on enzyme activity; based on mRNA there was CYP3A4 induction at 1 M, but not at lower concentrations. Metabolism, inhibition and induction were not observed with other CYPs. Potential DDI interactions exist with drugs that are inhibitors of CYP3A4-T or those metabolized by CYP3A4/3A5; these DDI risks will be better defined once there are human PK data.

Compound 1 was highly protein bound (95.6 to 98.7%) to plasma protein across species. Binding was independent of concentration.

In in vitro studies, metabolic stability was greatest in human hepatocytes and 22 metabolites were detected in human, monkey, dog, rat and/or mouse hepatocytes. Compound 2 was a major de-methylated active metabolite in all species and with the exception of a minor metabolite,

In in vivo studies in rat, dog and monkey, 35 metabolites were identified. The major circulating compound in all species was parent compound; parent was also a major component in rat feces. Compound 2, the de-methylated metabolite, was a major active metabolite in all species in plasma and in rat feces. Compound 1 was extensively metabolized during excretion with 31 metabolites in rat urine and 32 metabolites in rat feces.

### Brain and Plasma Exposure Studies

Comparative assessment of brain and plasma exposure of Compound 1, Compound 2 and gefitinib were performed.

TABLE 16
Comparative Assessment of Brain Exposure of
Compound 1 and Geftinib following Administration
to Sprague Dawley Rats (Single Oral PO)

| | | | | | Brain | |
|---|---|---|---|---|---|---|
| | | | Plasma | | Tissue | |
| | | | Concentration | | Concen- | Brain/ |
| | | | by Hour | | tration | Plasma |
| Test | Dose | Animal | (ng/mL) | | (ng/g) | (ratios) |
| Article | (mg/kg) | ID | 3 hrs | 7 hrs | 7 hrs | 7 hrs |
| Gefitinib | 50 mg/kg | 1M001 | 1260 | 1030 | 562 | 0.546 |
| | Gefitinib | 1M002 | 2660 | 2240 | 561 | 0.250 |
| | | 1M003 | 1940 | 4700 | 666 | 0.142 |
| | | Mean | 1953 | 2657 | 596 | 0.224 |
| | | SD | 700 | 1870 | 60.3 | 0.0322 |
| | | n | 3 | 3 | 3 | 3 |
| Com- | 30 mg/kg | 2M001 | 273 | 275 | 7400 | 26.9 |
| pound 1 | Com- | 2M002 | 181 | 265 | 8280 | 31.2 |
| | pound 1 | 2M003 | 229 | 197 | 5210 | 26.4 |
| | | Mean | 228 | 246 | 6960 | 28.3 |
| | | SD | 46 | 42 | 1580 | 3.73 |
| | | n | 3 | 3 | 3 | 3 |

TABLE 17
Comparative Assessment of Brain Exposure of Compound 11, Compound 12 and Geftinib
following Administration to Sprague Dawley Rats (Route (IV), 3 mg/kg)

| | | | | | | | Brain Tissue | Brain/ |
|---|---|---|---|---|---|---|---|---|
| | | Plasma Concentration by Hour | | | | | Concentration | Plasma |
| Test | Animal | (ng/mL) | | | | | (ng/g) | (ratios) |
| Article | ID | 0.083 | 1 | 2 | 3 | 5 | 5 | 5 |
| Gefitinib | 1M001 | 39.9 | 135 | 210 | 279 | 292 | 151 | 0.517 |
| | 1M002 | 38.2 | 125 | 166 | 206 | 282 | 149 | 0.528 |
| | 1M003 | 35.8 | 88.4 | 161 | 216 | 303 | 189 | 0.624 |
| | 1M004 | 55.6 | 115 | 161 | 225 | 286 | 155 | 0.542 |
| | 1M005 | 42.9 | 122 | 164 | 265 | 281 | 153 | 0.544 |
| | 1M006 | 39.8 | 116 | 180 | 252 | 237 | 129 | 0.544 |
| | Mean | 42.0 | 117 | 174 | 241 | 280 | 154 | 0.550 |
| | SD | 7.04 | 15.7 | 19.1 | 29.1 | 22.6 | 19.4 | 0.0377 |
| | n | 6 | 6 | 6 | 6 | 6 | 6 | 6 |
| Compound 1 | 2M007 | 14.3 | 63.0 | 70.1 | 120 | 119 | 2640 | 22.2 |
| | 2M008 | 14.2 | 63.5 | 93.4 | 95.5 | 107 | 2380 | 22.2 |
| | 2M009 | 13.5 | 54.8 | 84.2 | 93.7 | 120 | 2400 | 20.0 |
| | 2M010 | 12.8 | 59.6 | 81.6 | 106 | 109 | 2020 | 18.5 |
| | 2M011 | 13.1 | 44.2 | 70.7 | 89.5 | 97.4 | 2000 | 20.5 |
| | 2M012 | 11.3 | 51.1 | 71.9 | 94.7 | 109 | 1980 | 18.2 |
| | Mean | 13.2 | 56.0 | 78.7 | 99.9 | 110 | 2240 | 20.3 |
| | SD | 1.10 | 7.52 | 9.37 | 11.3 | 8.37 | 275 | 1.74 |
| | N | 6 | 6 | 6 | 6 | 6 | 6 | 6 |
| Compound 2 | 2M007 | BLQ | 3.24 | 5.62 | 12.9 | 18.2 | 24.5 | 1.35 |
| | 2M008 | BLQ | 3.47 | 7.75 | 12.9 | 18.1 | 23.6 | 1.30 |
| | 2M009 | BLQ | 3.14 | 6.99 | 10.6 | 19.6 | 20.9 | 1.07 |
| | 2M010 | BLQ | 3.47 | 8.41 | 12.7 | 18.9 | 21.0 | 1.11 |
| | 2M011 | BLQ | 3.28 | 7.39 | 12.5 | 20.4 | 21.6 | 1.06 |
| | 2M012 | BLQ | 2.71 | 6.20 | 12.3 | 16.8 | 19.8 | 1.18 |
| | Mean | BLQ | 3.22 | 7.06 | 12.3 | 18.7 | 21.9 | 1.18 |
| | SD | — | 0.281 | 1.02 | 0.873 | 1.26 | 1.79 | 0.123 |
| | N | 6 | 6 | 6 | 6 | 6 | 6 | 6 |

TABLE 18
Compound 1 Comparison between Plasma,
Brain and Lung in Sprague Dawley Rats (n = 4,
po, qd for 7 days, 24 hrs after last dosing)

| | Animal | Brain | Lung | Plasma |
|---|---|---|---|---|
| Group | No. | (ng/g) | (ng/g) | (ng/mL) |
| Control | Female | BLOG | BLOG | BLOG |
| | Male | BLOG | BLOG | BLOG |
| | Female | 39.76 | 1224.44 | 20.81 |
| Compound 1 | Male | 14.86 | 328.90 | 10.55 |
| (10 mg/kg) | | | | |
| Compound 1 | Female | 3774.45 | 44972.78 | 243.64 |
| (50 mg/kg) | Male | 2079.12 | 9707.15 | 149.31 |

### INCORPORATION BY REFERENCE

All of the U.S. patents, and U.S. and PCT published patent applications cited herein are hereby incorporated by reference.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

We claim:

1. A method of treating glioblastoma multiforme, astrocytoma, congenital tumor of the brain, ependymoma, germinoma, glioma, gliomatosis, gliosarcoma, medulloblastoma, meningioma, meningiosarcoma, oligodendroglioma, pinealoma, retinoblastoma, schwannoma, or spinal cord neurofibroma, comprising administering to a human subject in need thereof a therapeutically effective amount of a compound of Formula I: 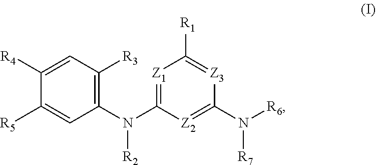
or pharmaceutically acceptable salts, hydrates, solvates, stereoisomers, or tautomers thereof, wherein the therapeutically effective amount is at least 100 mg/day, and wherein:
Z₁, Z₂, and Z₃ are each independently N or CR₈, wherein at least two of Z₁, Z₂, and Z₃ are N;
R₈ is H, (C₁-C₄) alkyl, (C₁-C₄) haloalkyl, or halogen;
R₁ is H, (C₁-C₄) alkyl, (C₁-C₄) haloalkyl, NH₂, NH(C₁-C₄) alkyl, N((C₁-C₄) alkyl)₂, or halogen;
R₂ is H or (C₁-C₆) alkyl;
R₃ is (C₁-C₄) alkoxy, (C₁-C₄) alkyl, (C₁-C₄) haloalkyl, or halogen;
R₄ is NR₉R₁₀ or a 5- to 7-membered heterocycle comprising 1-3 heteroatoms selected from N, O, and S and optionally substituted with one or more R₁₁;
R₉ is H or (C₁-C₄) alkyl;
R₁₀ is (C₁-C₄) alkyl, (C₁-C₄) alkyl-NH(C₁-C₄) alkyl, or (C₁C₄) alkyl-N((C₁-C₄) alkyl)₂;
or R₉ and R₁₀ together with the nitrogen atom to which they are attached form a 5- to 7-membered heterocycle optionally comprising 1 or 2 additional heteroatoms selected from N, O, and S and optionally substituted with one or more R₁₁;
each R₁ is independently (C₁-C₄) alkyl, (C₁-C₄) haloalkyl, (C₁-C₄) alkoxy, or halogen;
R₅ is NR₁₂C(O)R₁₃ or C(O)NR₁₂R₁₃;
R₁₂ is H or (C₁-C₆) alkyl;
R₁₃ is (C₁-C₆) alkyl or (C₂-C₆) alkenyl, wherein the alkyl or alkenyl is optionally substituted with one or more substituents independently selected from halogen, OH, CN, and NH₂;
R₆ and R₇ together with the nitrogen atom to which they are attached form a substituent of the formula, 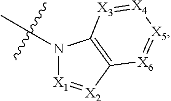
wherein
X₃ is N;
X₁, X₂, X₄, X₅ and X₆ are each independently CH or CR₁₅; and
each R₁₅ is independently (C₁-C₆) alkyl, (C₁-C₆) haloalkyl, (C₁-C₆) alkoxy, OH, NH₂, NH(C₁-C₆) alkyl, N((C₁-C₆) alkyl)₂, or halogen.

2. The method of claim 1, wherein the subject does not lose more than 10% of its body weight within 1 month after administration.

3. The method of claim 1, wherein the therapeutically effective amount of the compound is administered daily to the subject for at least 1 month; and subject does not lose more than 10% of its body weight within 1 month of daily administration.

4. The method of any one of claims 1-3, wherein the therapeutically effective amount is from 100 mg/day to 1000 mg/day.

5. The method of any one of claims 1-3, wherein the therapeutically effective amount is from 100 mg/day to 800 mg/day.

6. The method of any one of claims 1-3, wherein the therapeutically effective amount is from 100 mg/day to 500 mg/day.

7. The method of any one of claims 1-3, wherein the therapeutically effective amount is from 200 mg/day to 500 mg/day.

8. The method of any one of claim 1-7, further comprising examining the skin of the subject within 1 month after administration, wherein the subject does not exhibit skin lesions within 1 month after administration.

9. The method of any one of claim 1-7, further comprising examining the skin of the subject within 2 months after administration, wherein the subject does not exhibit skin lesions within 2 months after administration.

10. The method of any one of claim 1-9, wherein the method is a method of treating glioblastoma multiforme.

11. The method of claim 10, wherein the glioblastoma multiforme is characterized by elevated levels of EGFR and/or mutated EGFR.

12. The method of claim 10 or claim 11, wherein the compound of Formula I is not a substrate of an efflux transporter.

13. The method of claim 11, wherein the compound of Formula I is characterized by a binding affinity for EGFR and/or mutated EGFR in the subject of no more than 10 nM, such as no more than 9 nM, no more than 8 nM, no more than 7 nM, no more than 6 nM, no more than 5 nM, no more than 4 nM, no more than 3 nM, no more than 2 nM, no more than 1 nM, no more than 0.9 nM, no more than 0.8 nM, no more than 0.7 nM, no more than 0.6 nM, no more than 0.5 nM, no more than 0.4 nM, no more than 0.3 nM, no more than 0.2 nM, no more than 0.15 nM, no more than 0.12 nM, no more than 0.11 nM, or no more than 0.10 nM.

14. The method of any one of claim 1-9, wherein the method is a method of treating an astrocytoma.

15. The method of any one of claims 1-14, wherein Z₁ and Z₂ are each N and Z₃ is CR₈.

16. The method of any one of claims 1-15, wherein R₁ is H or NH₂.

17. The method of any one of claims 1-16, wherein R₂ is H.

18. The method of any one of claims 1-17, wherein R₃ is (C₁-C₄) alkoxy.

19. The method of any one of claims 1-18, wherein R₄ is NR₉R₁₀.

20. The method of any one of claims 1-19, wherein R₅ is NR₁₂C(O)R₁₃.

21. The method of any one of claims 1-20, wherein R₁₅ is selected from (C₁-C₆) alkyl and (C₁-C₆) haloalkyl.

22. The method of claim 21, wherein R₁₅ is selected from methyl and CF₃.

23. The method of any one of claims 1-22, wherein R₈ is H or halogen.

24. The method of any one of claims 1-23, wherein R₉ is (C₁-C₄) alkyl.

25. The method of any one of claims 1-24, wherein R₁₀ is (C₁-C₄) alkyl-NH(C₁-C₄) alkyl, or (C₁-C₄) alkyl-N((C₁-C₄) alkyl)₂.

26. The method of any one of claims 1-25, wherein R₄ is NR₉R₁₀ and R₉ and R₁₀ together with the nitrogen atom to which they are attached form a 5- to 7-membered heterocycle optionally comprising 1 or 2 additional heteroatoms selected from N, O, and S and optionally substituted with one or more R₁₁.

27. The method of any one of claims 1-26, wherein R₁₁ is (C₁-C₄) alkyl, R₁₂ is H, and R₁₃ is (C₂-C₆) alkenyl.

28. The method of any one of claims 1-26, wherein Ru is (C₁-C₄) alkyl, R₁₂ is (C₁-C₆) alkyl, and R₁₃ is (C₂-C₆) alkenyl.

29. The method of any one of claims 1-14, wherein the compound of Formula I is a compound of Formula Ia: 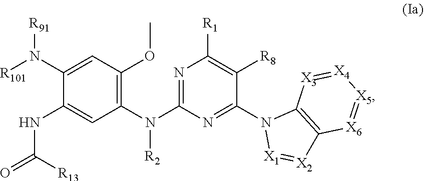
or pharmaceutically acceptable salts, hydrates, solvates, stereoisomers, or tautomers thereof, wherein:
X₁, X₂, X₄, X₅ and X₆ are each independently CR₁₅;
R₉₁ is (C₁-C₄) alkyl;
R₁₀₁ is (C₁-C₄) alkyl-NH(C₁-C₄) alkyl or (C₁-C₄) alkyl-N((C₁-C₄) alkyl)₂;
or R₉₁ and R₁₀₁ together with the nitrogen atom to which they are attached form a 5- to 7-membered heterocycle optionally comprising 1 or 2 additional heteroatoms selected from N, O, and S and optionally substituted with one or more Rn.

30. The method of any one of claims 1-14, wherein the compound is selected from the group consisting of:
N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide;
N-(2-((2-(dimethylamino)ethyl)(methyl)amino)-4-methoxy-5-((4-(3-(trifluoromethyl)-1H-pyrrolo[2,3-b]pyridin-1-yl)pyrimidin-2-yl)amino)phenyl)acrylamide;
N-(4-methoxy-2-(4-methylpiperazin-1-yl)-5-((4-(3-(trifluoromethyl)-1H-pyrrolo[2,3-b]pyridin-1-yl)pyrimidin-2-yl)amino)phenyl)acrylamide;
N-(5-((4-(1H-pyrrolo[2,3-b]pyridin-1-yl)pyrimidin-2-yl)amino)-4-methoxy-2-(4-methylpiperazin-1-yl)phenyl)acrylamide; and
N-(2-((2-(dimethylamino)ethyl)(methyl)amino)-4-methoxy-5-((4-(3-methyl-1H-pyrrolo[2,3-b]pyridin-1-yl) pyrimidin-2-yl)amino)phenyl)acrylamide,
or a pharmaceutically acceptable salt thereof.

31. The method of any one of claims 1-14, wherein the compound is N-(5-((4-(1H-pyrrolo[2,3-b]pyridin-1-yl)pyrimidin-2-yl)amino)-2-((2-(dimethylamino)ethyl)(methyl)amino)-4-methoxyphenyl)acrylamide, or a pharmaceutically acceptable salt thereof.

32. The method of any one of claims 1-31, wherein the compound is administered once per day.

33. The method of any one of claims 1-31, wherein the compound is administered two times per day.

34. The method of any one of claims 1-31, wherein the compound is administered three times per day.

35. The method of any one of claims 1-34, wherein the compound is administered systemically.

36. The method of claim 35, wherein the compound is administered orally.

37. The method of claim 35, wherein the compound is administered intravenously.

38. A method for treating or reducing a brain tumor, or a related disease or condition, comprising administering to a subject in need thereof a therapeutically effective amount of a compound having the formula of Compound 1: 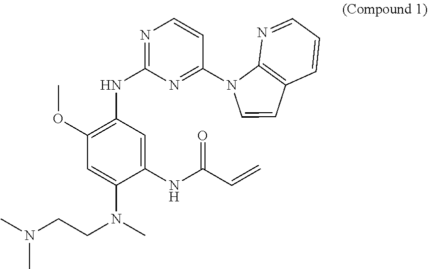 [SMILES: C=CC(=O)Nc1cc(Nc2nccc(-n3ccc4cccnc43)n2)c(OC)cc1N(C)CCN(C)C]
or a pharmaceutically acceptable form or an isotope derivative thereof.

39. A method for inhibiting or reducing the activity of epidermal growth factor receptor (EGFR) in a subject suffering from a brain tumor, comprising administering to the subject a therapeutically effective amount of a compound having the formula of Compound 1: 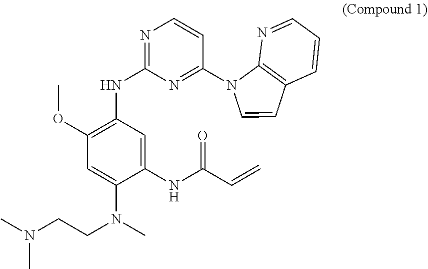 [SMILES: C=CC(=O)Nc1cc(Nc2nccc(-n3ccc4cccnc43)n2)c(OC)cc1N(C)CCN(C)C]
or a pharmaceutically acceptable form or an isotope derivative thereof.

40. A method for treating or reducing a brain disease or condition mediated by epidermal growth factor receptor (EGFR), comprising administering to the subject in need thereof a therapeutically effective amount of a compound having the formula of Compound 1: 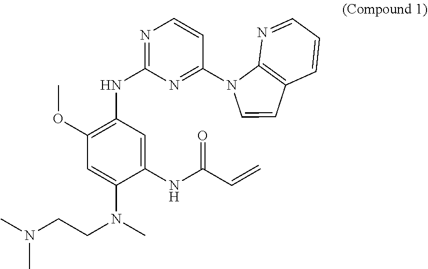 [SMILES: C=CC(=O)Nc1cc(Nc2nccc(-n3ccc4cccnc43)n2)c(OC)cc1N(C)CCN(C)C]
or a pharmaceutically acceptable form or an isotope derivative thereof.

41. The method of any one of claims 38-40, wherein the brain tumor comprises a primary tumor.

42. The method of any one of claims 38-40, wherein the brain tumor comprises a metastatic tumor.

43. The method of claim 41, wherein the brain tumor is glioblastoma.

44. The method of any one of claims 38-43, wherein the therapeutically effective amount is in the range from about 0.1 to about 20 mg/kg body weight daily.

45. The method of claim 44, wherein the therapeutically effective amount is in the range from about 0.5 to about 5 mg/kg body weight daily.

46. A pharmaceutical composition for treating a brain tumor, or a related disease or condition, comprising a compound having the formula of Compound 1: 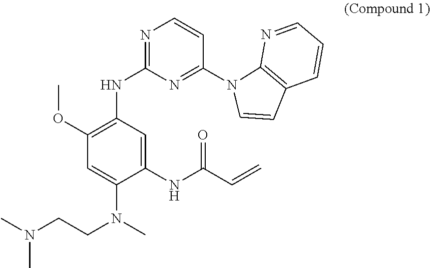 [SMILES: C=CC(=O)Nc1cc(Nc2nccc(-n3ccc4cccnc43)n2)c(OC)cc1N(C)CCN(C)C]
or a pharmaceutically acceptable form or an isotope derivative thereof.

47. The pharmaceutical composition of claim 46, wherein the brain tumor comprises a primary tumor.

48. The pharmaceutical composition of claim 46, wherein the brain tumor comprises a metastatic tumor.

49. The pharmaceutical composition of claim 47, wherein the brain tumor is glioblastoma.

50. A compound having the structural formula of Compound 2: 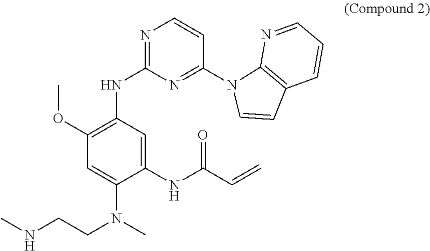 [SMILES: C=CC(=O)Nc1cc(Nc2nccc(-n3ccc4cccnc43)n2)c(OC)cc1N(C)CCNC]
or a pharmaceutically acceptable form or an isotope derivative thereof.

51. A pharmaceutical composition comprising a compound having the structural formula of Compound 2 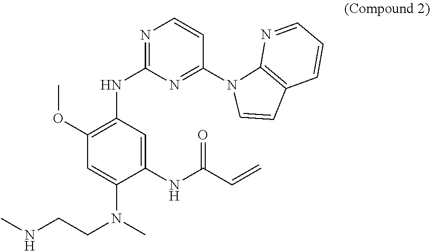 [SMILES: C=CC(=O)Nc1cc(Nc2nccc(-n3ccc4cccnc43)n2)c(OC)cc1N(C)CCNC]
or a pharmaceutically acceptable form or an isotope derivative thereof, and a pharmaceutically acceptable excipient, carrier, or diluent.

52. The pharmaceutical composition of claim 51, being suitable for oral administration.

53. The pharmaceutical composition of claim 51, being suitable for intravenous administration.

54. The pharmaceutical composition of any one of claims 51-53, suitable for use in treating a disease or condition selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas and myelomas.

55. A unit dosage form comprising a pharmaceutical composition of any one of claims 51-54.

56. A method for treating or reducing a disease or condition, comprising administering to a subject in need thereof a therapeutically effective amount of a compound having the formula of Compound 2: 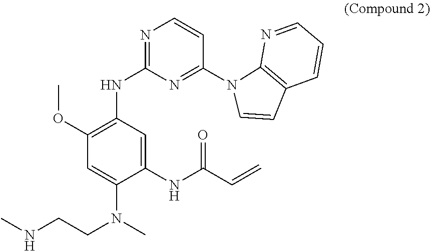 [SMILES: C=CC(=O)Nc1cc(Nc2nccc(-n3ccc4cccnc43)n2)c(OC)cc1N(C)CCNC]
or a pharmaceutically acceptable form or an isotope derivative thereof.

57. A method for inhibiting or reducing the activity of epidermal growth factor receptor (EGFR) in a subject suffering from a disease or condition related thereto, comprising administering to the subject a therapeutically amount of a compound having the formula of Compound 2: 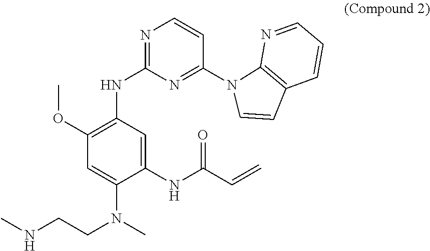 [SMILES: C=CC(=O)Nc1cc(Nc2nccc(-n3ccc4cccnc43)n2)c(OC)cc1N(C)CCNC]
or a pharmaceutically acceptable form or an isotope derivative thereof.

58. A method for treating or reducing a disease or condition mediated by epidermal growth factor receptor (EGFR), comprising administering to the subject in need thereof a therapeutically effective amount of a compound having the formula of Compound 2: 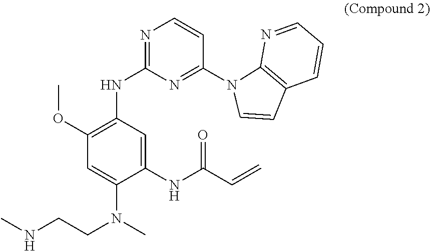 [SMILES: C=CC(=O)Nc1cc(Nc2nccc(-n3ccc4cccnc43)n2)c(OC)cc1N(C)CCNC]
or a pharmaceutically acceptable form or an isotope derivative thereof.

59. The method of any one of claims 56-58, wherein the disease or condition is a cancer.

60. The method of any one of claims 56-59, wherein the cancer is selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas and myelomas.

61. The method of claim 60, wherein the cancer comprises a primary tumor.

62. The method of claim 60, wherein the cancer comprises a metastatic tumor.

63. The method of claim 60, wherein the cancer is glioblastoma.

64. The method of claim 60, wherein the cancer is lung cancer.

65. The method of claim 64, wherein the cancer is non-small cell lung cancer (NSCLC).

66. The method of claim 64, wherein the cancer is small cell lung cancer (SCLC).

67. The methods of any one of claims 56-66, wherein the subject carries an EGFR mutation.

68. The method of claim 67, wherein the subject carries T790M EGFR mutation.

69. The method of any one of claims 58-68, wherein the therapeutically effective amount is in the range from about 0.1 to about 20 mg/kg body weight daily.

70. The method of claim 69, wherein the therapeutically effective amount is in the range from about 0.5 to about 5 mg/kg body weight daily.

71. A method for treating or reducing a brain tumor, or a related disease or condition, comprising administering to a subject in need thereof a therapeutically effective amount of a compound having the formula of Compound 2: 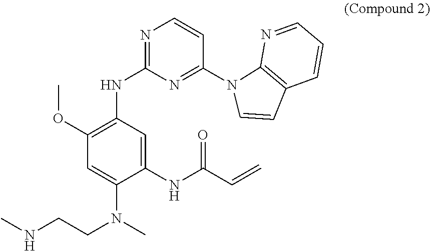 [SMILES: C=CC(=O)Nc1cc(Nc2nccc(-n3ccc4cccnc43)n2)c(OC)cc1N(C)CCNC]
or a pharmaceutically acceptable form or an isotope derivative thereof.

72. A method for inhibiting or reducing the activity of epidermal growth factor receptor (EGFR) in a subject suffering from a brain tumor, comprising administering to a subject in need thereof a therapeutically amount of a compound having the formula of Compound 2: 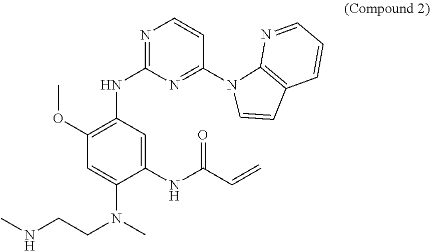 [SMILES: C=CC(=O)Nc1cc(Nc2nccc(-n3ccc4cccnc43)n2)c(OC)cc1N(C)CCNC]
or a pharmaceutically acceptable form or an isotope derivative thereof.

73. A method for treating or reducing a brain disease or condition mediated by epidermal growth factor receptor (EGFR), comprising administering to the subject in need thereof a therapeutically effective amount of a compound having the formula of Compound 2: 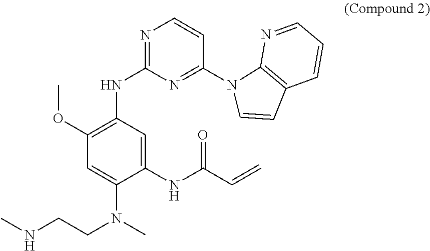 [SMILES: C=CC(=O)Nc1cc(Nc2nccc(-n3ccc4cccnc43)n2)c(OC)cc1N(C)CCNC]
or a pharmaceutically acceptable form or an isotope derivative thereof.

74. The method of claim 73, wherein the brain tumor comprises a primary tumor.

75. The method of claim 73, wherein the brain tumor comprises a metastatic tumor.

76. The method of any one of claims 71-73, wherein the brain tumor is glioblastoma.

77. Use of a compound or a pharmaceutical composition thereof for treating or reducing a brain tumor, or a related disease or condition, wherein the compound has the formula of Compound 1: 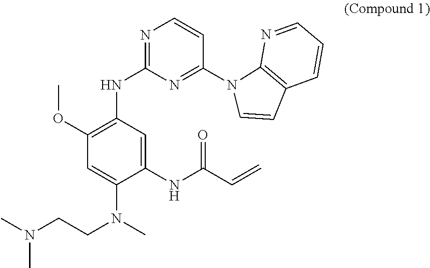 [SMILES: C=CC(=O)Nc1cc(Nc2nccc(-n3ccc4cccnc43)n2)c(OC)cc1N(C)CCN(C)C]
or a pharmaceutically acceptable form or an isotope derivative thereof.

78. Use of a compound or a pharmaceutical composition thereof for inhibiting or reducing the activity of epidermal growth factor receptor (EGFR) in a subject suffering from a brain tumor, wherein the compound has the formula of Compound 1: 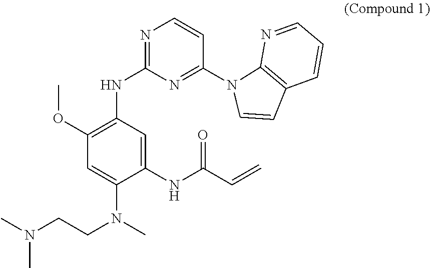 [SMILES: C=CC(=O)Nc1cc(Nc2nccc(-n3ccc4cccnc43)n2)c(OC)cc1N(C)CCN(C)C]
or a pharmaceutically acceptable form or an isotope derivative thereof.

79. Use of a compound or a pharmaceutical composition thereof for treating or reducing a brain disease or condition mediated by epidermal growth factor receptor (EGFR), comprising administering to the subject in need thereof a therapeutically effective amount of a compound having the formula of Compound 1: 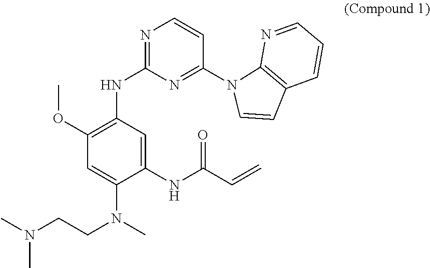 [SMILES: C=CC(=O)Nc1cc(Nc2nccc(-n3ccc4cccnc43)n2)c(OC)cc1N(C)CCN(C)C]
or a pharmaceutically acceptable form or an isotope derivative thereof.

80. Use of any one of claims 77-79, wherein the brain tumor comprises a primary tumor.

81. Use of any one of claims 77-79, wherein the brain tumor comprises a metastatic tumor.

82. Use of any one of claims 77-79, wherein the brain tumor is glioblastoma.

83. Use of a compound or a pharmaceutical composition thereof for treating or reducing a disease or condition, wherein the compound has the formula of Compound 2: 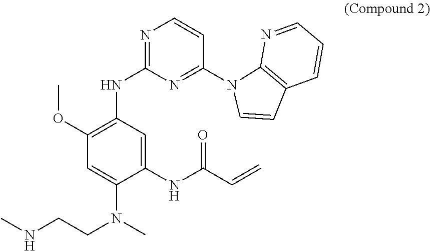 [SMILES: C=CC(=O)Nc1cc(Nc2nccc(-n3ccc4cccnc43)n2)c(OC)cc1N(C)CCNC]
or a pharmaceutically acceptable form or an isotope derivative thereof.

84. Use of a compound or a pharmaceutical composition thereof for inhibiting or reducing the activity of epidermal growth factor receptor (EGFR) in a subject suffering from a disease or condition related thereto, wherein the compound has the formula of Compound 2: 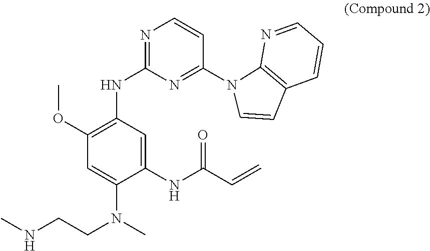 [SMILES: C=CC(=O)Nc1cc(Nc2nccc(-n3ccc4cccnc43)n2)c(OC)cc1N(C)CCNC]
or a pharmaceutically acceptable form or an isotope derivative thereof.

85. Use of a compound or a pharmaceutical composition thereof for treating or reducing a disease or condition mediated by epidermal growth factor receptor (EGFR), wherein the compound has the formula of Compound 2: 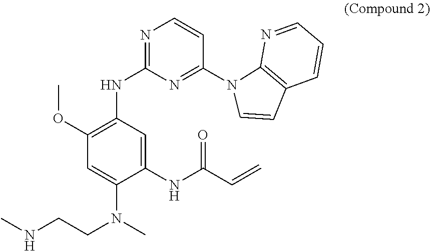 [SMILES: C=CC(=O)Nc1cc(Nc2nccc(-n3ccc4cccnc43)n2)c(OC)cc1N(C)CCNC]
or a pharmaceutically acceptable form or an isotope derivative thereof.

86. Use of any one of claims 83-85, wherein the disease or condition is a cancer.

87. Use of claim 86, wherein the cancer is selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas and myelomas.

88. Use of claim 87, wherein the brain tumor comprises a primary tumor.

89. Use of claim 87, wherein the brain tumor comprises a metastatic tumor.

90. Use of claim 87, wherein the cancer is glioblastoma.

91. Use of claim 87, wherein the cancer is lung cancer.

92. Use of claim 91, wherein the cancer is non-small cell lung cancer (NSCLC).
